(19) United States
(12) Patent Application Publication
(10) Pub. No.: US 2021/0253575 A1
(43) Pub. Date: Aug. 19, 2021

(54) PYRROLIDINE AMINE COMPOUNDS FOR THE TREATMENT OF AUTOIMMUNE DISEASE

(71) Applicant: Hoffmann-La Roche Inc., Little Falls, NJ (US)

(72) Inventors: Fabian Dey, Basel (CH); Haixia Liu, Shanghai (CN); Hong Shen, Shanghai (CN); Guolong Wu, Shanghai (CN); Weixing Zhang, Shanghai (CN); Wei Zhu, Shanghai (CN)

(73) Assignee: Hoffmann-La Roche Inc., Little Falls, NJ (US)

(21) Appl. No.: 17/274,415

(22) PCT Filed: Sep. 7, 2018
(86) PCT No.: PCT/EP2018/074078

§ 371 Date: Mar. 8, 2021

(51) Int. Cl. C07D 471/10; C07D 403/10; C07D 487/10; C07D 401/14; C07D 401/10; C07D 487/04; C07D 403/14; C07D 498/10; C07D 471/08

(52) U.S. Cl. CPC C07D 471/10; C07D 403/10; C07D 487/10; C07D 401/14; C07D 471/08; C07D 487/04; C07D 403/14; C07D 498/10; C07D 401/10

(57) ABSTRACT

The present invention relates to compounds of formula (I), wherein R¹, R², R³ and R⁴ are as described herein, and their pharmaceutically acceptable salt, enantiomer or diastereomer thereof, and compositions including the compounds and methods of using the compounds.

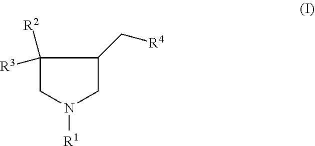

The present invention relates to organic compounds useful for therapy and/or prophylaxis in a mammal, and in particular to antagonist of TLR7 and/or TLR8 and/or TLR9 useful for treating systemic lupus erythematosus or lupus nephritis.

### FIELD OF THE INVENTION

Autoimmune connective tissue disease (CTD) include prototypical autoimmune syndromes such as Systemic Lupus Erythematosus (SLE), primary Sjögren's syndrome (pSjS), mixed connective tissue disease (MCTD), Dermatomyositis/Polymyositis (DM/PM), Rheumatoid Arthritis (RA), and systemic sclerosis (SSc). With the exception of RA, no really effective and safe therapies are available to patients. SLE represents the prototypical CTD with a prevalence of 20-150 per 100,000 and causes broad inflammation and tissue damage in distinct organs, from commonly observed symptoms in the skin and joints to renal, lung, or heart failure. Traditionally, SLE has been treated with nonspecific anti-inflammatory or immunosuppressive drugs. However, long term usage of immunosuppressive drug, e.g. corticosteroids is only partially effective, and is associated with undesirable toxicity and side effects. Belimumab is the only FDA-approved drug for lupus in the last 50 years, despite its modest and delayed efficacy in only a fraction of SLE patients (Navarra, S. V. et al Lancet 2011, 577, 721.). Other biologies, such as anti-CD20 mAbs, mAbs against or soluble receptors of specific cytokines, have failed in most clinical studies. Thus, novel therapies are required that provide sustained improvement in a greater proportion of patient groups and are safer for chronic use in many autoimmune as well as auto-inflammation diseases.

Toll Like Receptors (TLR) are an important family of pattern recognition receptors (PRR) which can initiate broad immune responses in a wide variety of immune cells. As natural host defense sensors, endosomal TLRs 7, 8 and 9 recognize nucleic acids derived from viruses, bacteria; specifically, TLR7/8 and TLR9 recognize single-stranded RNA (ssRNA) and single-stranded CpG-DNA, respectively. However, aberrant nucleic acid sensing of TRL7,8,9 is considered as a key node in a broad of autoimmune and auto-inflammatory diseases (Krieg, A. M. et al. Immunol. Rev. 2007, 220, 251. Jimenez-Dalmaroni, M. J. et al Autoimmun Rev. 2016, 15, 1. Chen, J. Q., et al. Clinical Reviews in Allergy & Immunology 2016, 50, 1.) Therefore, TLR7,8,9 represents a new therapeutic target for autoimmune and auto-inflammatory diseases, for which no effective steroid-free and non-cytotoxic oral drugs exist, and inhibition of these pathways from the very upstream may deliver satisfying therapeutic effects. From a safety perspective, because there are multiple nucleic acid sensing pathways (e.g. other TLRs, cGAS/STING), such redundancy should still allow responses to infection in the presence of TLR789 inhibition. As such, we proposed and invented oral compounds that target and suppress TLR7,8,9 for the treatment of autoimmune and auto-inflammatory diseases.

### SUMMARY OF THE INVENTION

The present invention relates to novel compounds of formula (I),

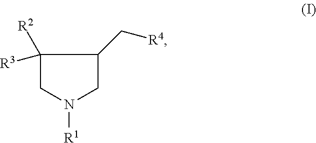

wherein
R¹ is

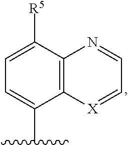

wherein
R₅ is cyano, C₁₋₆alkyl, halogen, haloC₁₋₆alkyl or nitro;
X is N or CH;
R² and R³ are independently selected from H, C₁₋₆alkyl, C₃₋₇cycloalkyl and haloC₁₋₆alkyl; or R² and R³ together with the carbon they are attached to form C₃₋₇cycloalkyl;
R⁴ is heterocyclyl or heterocyclylamino;
   or a pharmaceutically acceptable salt, enantiomer or diastereomer thereof.

Another object of the present invention is related to novel compounds of formula (I), their manufacture, medicaments based on a compound in accordance with the invention and their production as well as the use of compounds of formula (I) as TLR7 and/or TLR8 and/or TLR9 antagonist, and for the treatment or prophylaxis of systemic lupus erythematosus or lupus nephritis. The compounds of formula (I) show superior TLR7 and/or TLR8 and/or TLR9 antagonism activity. In addition, the compounds of formula (I) also show good cytotoxicity, solubility, human microsome stability and SDPK profiles, as well as low CYP inhibition.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, difluoromethyl, trifluoromethyl and trifluoroethyl.

The term “C₃₋₇cycloalkyl” denotes a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” groups are cyclopropyl and cyclohexyl.

The term “halopiperidinyl” denotes a piperidinyl group wherein at least one of the hydrogen atoms of the piperidinyl group has been replaced by same or different halogen atoms, particularly fluoro atoms. Examples of halopyrrolidinyl include fluoropiperidinyl and difluoropiperidinyl.

The term “heterocyclyl” denotes a monovalent saturated or partly unsaturated mono- or bicyclic ring system of 3 to 12 ring atoms, comprising 1, 2, or 3 ring heteroatoms selected from N, O and S, the remaining ring atoms being carbon. In particular embodiments, heterocyclyl is a monovalent saturated monocyclic ring system of 4 to 10 ring atoms, comprising 1, 2, or 3 ring heteroatoms selected from N, O and S, the remaining ring atoms being carbon. Examples for monocyclic saturated heterocyclyl are aziridinyl, oxiranyl, azetidinyl, oxetanyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, oxazepanyl, diazepanyl, homopiperazinyl, or oxazepanyl. Examples for bicyclic heterocyclyl are 2,3,3a,4,6,6a-hexahydro-1H-pyrrolo[3,4-c]pyrrolyl; azabicyclo[3.2.1]octanyl; azaspiro[3.3]heptanyl; diazaspiro[4.4]nonanyl; diazabicyclo[2.2.2]octanyl; diazabicyclo[4.2.0]octanyl; diazaspiro[3.5]nonanyl; diazaspiro[4.4]nonanyl; diazaspiro[4.5]decanyl; diazaspiro[5.5]undecanyl; oxadiazaspiro[5.5]undecanyl. Examples for partly unsaturated heterocyclyl are dihydrofuryl, imidazolinyl, dihydro-oxazolyl, tetrahydropyridinyl, and dihydropyranyl. Monocyclic or bicyclic heterocyclyl can be further substituted by halogen, hydroxy, amino, C₁₋₆alkyl, haloC₁₋₆alkyl, (C₁₋₆alkyl)₂aminoC₁₋₆alkyl, (C₁₋₆alkyl)₂amino, aminoC₁₋₆alkyl, C₁₋₆alkylaminoC₁₋₆alkyl, carbamoyl or heterocyclyl.

The term “enantiomer” denotes two stereoisomers of a compound which are non-superimposable mirror images of one another.

The term “diastereomer” denotes a stereoisomer with two or more centers of chirality and whose molecules are not mirror images of one another. Diastereomers have different physical properties, e.g. melting points, boiling points, spectral properties, and reactivities.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Antagonist of TLR7 and/or TLR8 and/or TLR9

The present invention relates to (i) a compound of formula (I),

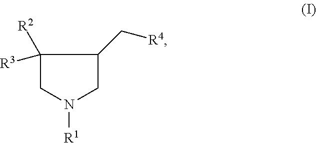

wherein
R¹ is

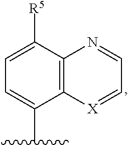

wherein
   R⁵ is cyano, C₁₋₆alkyl, halogen, haloC₁₋₆alkyl or nitro;
   X is N or CH;
R² and R³ are independently selected from H, C₁₋₆alkyl, C₃₋₇cycloalkyl and haloC₁₋₆alkyl; or R² and R³ together with the carbon they are attached to form C₃₋₇cycloalkyl;
R⁴ is heterocyclyl or heterocyclylamino;
   or a pharmaceutically acceptable salt, enantiomer or diastereomer thereof.

A further embodiment of present invention relates to (i′) a compound of formula (Ia),

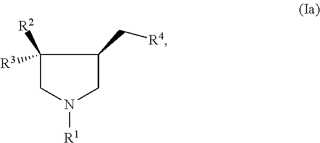

wherein
R¹ is

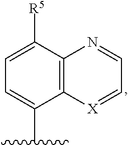

wherein
   R⁵ is cyano, C₁₋₆alkyl, halogen, haloC₁₋₆alkyl or nitro;
   X is N or CH;
R² and R³ are independently selected from H, C₁₋₆alkyl, C₃₋₇cycloalkyl and haloC₁₋₆alkyl; or R² and R³ together with the carbon they are attached to form C₃₋₇cycloalkyl;
R⁴ is heterocyclyl or heterocyclylamino;
   or a pharmaceutically acceptable salt, enantiomer or diastereomer thereof.

A further embodiment of present invention is (ii) a compound of formula (I) or (Ia), wherein
wherein
R¹ is

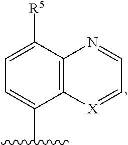

wherein
   R⁵ is cyano or haloC₁₋₆alkyl;
   X is N or CH;
R² is H;
R³ is H, C₁₋₆alkyl, C₃₋₇cycloalkyl or haloC₁₋₆alkyl;
   or R² and R³ together with the carbon they are attached to form C₃₋₇cycloalkyl;
R⁴ is heterocyclyl or heterocyclylamino;
   or a pharmaceutically acceptable salt, enantiomer or diastereomer thereof.

A further embodiment of present invention is (iii) a compound of formula (I) according to (ii), wherein
R⁴ is 2,3,3a,4,6,6a-hexahydro-1H-pyrrolo[3,4-c]pyrrolyl;
   aminoazabicyclo[3.2.1]octanyl;
   aminoazaspiro[3.3]heptanyl;
   azepanylamino;
   C₁₋₆alkyldiazaspiro[4.4]nonanyl;
   diazabicyclo[2.2.2]octanyl;
   diazabicyclo[4.2.0]octanyl;
   diazaspiro[3.5]nonanyl;
   diazaspiro[4.4]nonanyl;
   diazaspiro[4.5]decanyl;
   diazaspiro[5.5]undecanyl;
   oxadiazaspiro[5.5]undecanyl;
   piperazinyl;
   piperidinyl, said piperidinyl being substituted by one, two or three substituents independently selected from amino, halogen, C₁₋₆alkyl, aminoC₁₋₆alkyl, (C₁₋₆alkyl)₂amino, C₁₋₆alkylaminoC₁₋₆alkyl, carbamoyl, azepanyl, morpholinyl, piperidinyl, piperazinyl and pyrrolidinyl;
   piperidinylamino; or
   pyrrolidinyl, said pyrrolidinyl being substituted by one, two or three substituents independently selected from C₁₋₆alkyl, (C₁₋₆alkyl)₂aminoC₁₋₆alkyl, (C₁₋₆alkyl)₂amino and aminoC₁₋₆alkyl.

A further embodiment of present invention is (iv) a compound of formula (I) according to (iii), wherein R⁴ is 2,3,3a,4,6,6a-hexahydro-1H-pyrrolo[3,4-c]pyrrolyl; aminoazabicyclo[3.2.1]octanyl; aminoazaspiro[3.3]heptanyl; azepanylamino; C₁₋₆alkyldiazaspiro[4.4]nonanyl; diazabicyclo[2.2.2]octanyl; diazabicyclo[4.2.0]octanyl; diazaspiro[3.5]nonanyl; diazaspiro[4.4]nonanyl; diazaspiro[4.5]decanyl; diazaspiro[5.5]undecanyl; oxadiazaspiro[5.5]undecanyl; piperazinyl; amino(C₁₋₆alkyl)piperidinyl; piperidinylpiperidinyl; aminopiperidinyl; piperazinylpiperidinyl; morpholinylpiperidinyl; pyrrolidinylpiperidinyl; azepanylpiperidinyl; aminohalopiperidinyl; carbamoylpiperidinyl; (aminoC₁₋₆alkyl)piperidinyl; aminoC₁₋₆alkyl(C₁₋₆alkyl)piperidinyl; (aminoC₁₋₆alkyl)halopiperidinyl; (C₁₋₆alkyl)₂aminopiperidinyl; C₁₋₆alkylaminoC₁₋₆alkylpiperidinyl; piperidinylamino; aminoC₁₋₆alkyl(C₁₋₆alkyl)pyrrolidinyl; (C₁₋₆alkyl)₂aminopyrrolidinyl; or (C₁₋₆alkyl)₂aminoC₁₋₆alkylpyrrolidinyl.

A further embodiment of present invention is (v) a compound of formula (I) according to (iv), wherein R⁵ is cyano or trifluoromethyl.

A further embodiment of present invention is (vi) a compound of formula (I) according to (v), wherein R³ is H, methyl, ethyl, isopropyl, difluoromethyl, trifluoromethyl or cyclopropyl; or R² and R³ together with the carbon they are attached to form cyclopropyl.

A further embodiment of present invention is (vii) a compound of formula (I) according to (vi), wherein R³ is methyl or trifluoromethyl; or R² and R³ together with the carbon they are attached to form cyclopropyl.

A further embodiment of present invention is (viii) a compound of formula (I) according to (v) or (vi), wherein R⁴ is (dimethylamino)methylpyrrolidinyl; (dimethylamino)pyrrolidinyl; 1,9-diazaspiro[5.5]undecan-9-yl; l-oxa-4,9-diazaspiro[5.5]undecan-4-yl; l-oxa-4,9-diazaspiro[5.5]undecan-9-yl; 2,3,3a,4,6,6a-hexahydro-1H-pyrrolo[3,4-c]pyrrol-5-yl; 2,5-diazabicyclo[2.2.2]octan-2-yl; 2,7-diazaspiro[3.5]nonan-2-yl; 2,7-diazaspiro[4.4]nonan-2-yl; 2,8-diazaspiro[3.5]nonan-2-yl; 2,8-diazaspiro[4.5]decan-2-yl; 2,8-diazaspiro[4.5]decan-8-yl; 2,9-diazaspiro[4.5]decan-2-yl; 2,9-diazaspiro[5.5]undecan-2-yl; 2,9-diazaspiro[5.5]undecan-9-yl; 3-(aminomethyl)-3-fluoro-1-piperidinyl; 3-(aminomethyl)-3-methyl-1-piperidinyl; 3-(aminomethyl)-3-methyl-pyrrolidin-1-yl; 3,7-diazabicyclo[4.2.0]octan-3-yl; 3,8-diazabicyclo[4.2.0]octan-8-yl; 3,9-diazaspiro[5.5]undecan-3-yl; 3-amino-8-azabicyclo[3.2.1]octan-8-yl; 4-(1-piperidinyl)-1-piperidinyl; 4-(2-aminoethyl)-1-piperidinyl; 4-(aminomethyl)-1-piperidinyl; 4-(azepan-1-yl)-1-piperidinyl; 4-(dimethylamino)-1-piperidinyl; 4-(methylaminomethyl)-1-piperidinyl; 4-amino-1-piperidinyl; 4-amino-3,3-difluoro-1-piperidinyl; 4-amino-3-methyl-1-piperidinyl; 4-amino-4-methyl-1-piperidinyl; 4-morpholino-1-piperidinyl; 4-piperazin-1-yl-1-piperidinyl; 4-pyrrolidin-1-yl-1-piperidinyl; 6-amino-2-azaspiro[3.3]heptan-2-yl; 7-methyl-2,7-diazaspiro[4.4]nonan-2-yl; 8-amino-3-azabicyclo[3.2.1]octan-3-yl; azepan-4-ylamino; carbamoylpiperidinyl; piperazinyl or piperidinylamino.

A further embodiment of present invention is (viii) a compound of formula (I) according to (viii), wherein R⁴ is 2,7-diazaspiro[3.5]nonan-2-yl; 1,9-diazaspiro[5.5]undecan-9-yl; 4-amino-1-piperidinyl; 4-amino-1-piperidinyl or piperidinylamino.

Another embodiment of present invention is that (x) particular compounds of formula (I) are selected from the following:
8-[(3S,4R)-3-(3,9-diazaspiro[5.5]undecan-3-ylmethyl)-4-(trifluoromethyl)pyrrolidin-1-yl]quinoxaline-5-carbonitrile;
5-[(3S,4R)-3-(3,9-diazaspiro[5.5]undecan-3-ylmethyl)-4-(trifluoromethyl)pyrrolidin-1-yl]quinoline-8-carbonitrile;
8-[7-(piperazin-1-ylmethyl)-5-azaspiro[2.4]heptan-5-yl]quinoxaline-5-carbonitrile;
8-[7-[(8-Amino-3-azabicyclo[3.2.1]octan-3-yl)methyl]-5-azaspiro[2.4]heptan-5-yl]quinoxaline-5-carbonitrile;
8-[(3S,4R)-3-(2,7-diazaspiro[4.4]nonan-2-yl methyl)-4-(trifluoromethyl)pyrrolidin-1-yl]quinoxaline-5-carbonitrile;
5-[(3S,4R)-3-(2,9-diazaspiro[4.5]decan-2-ylmethyl)-4-(trifluoromethyl)pyrrolidin-1-yl]quinoline-8-carbonitrile;
8-[7-[(4-Amino-4-methyl-1-piperidyl)methyl]-5-azaspiro[2.4]heptan-5-yl]quinoxaline-5-carbonitrile;
5-[(3S,4R)-3-(2,8-diazaspiro[3.5]nonan-2-yl methyl)-4-(trifluoromethyl)pyrrolidin-1-yl]quinoline-8-carbonitrile;
5-[(3S,4R)-3-(2,7-diazaspiro[4.4]nonan-2-yl methyl)-4-(trifluoromethyl)pyrrolidin-1-yl]quinoline-8-carbonitrile;
5-[(3S,4R)-3-[[4-(1-piperidyl)-1-piperidyl]methyl]-4-(trifluoromethyl)pyrrolidin-1-yl]quinoline-8-carbonitrile;
8-[7-[[4-(1-Piperidyl)-1-piperidyl]methyl]-5-azaspiro[2.4]heptan-5-yl]quinoxaline-5-carbonitrile;
8-[(3S,4R)-3-[(4-amino-4-methyl-1-piperidyl)methyl]-4-(trifluoromethyl)pyrrolidin-1-yl]quinoxaline-5-carbonitrile;
8-[7-(2,9-Diazaspiro[5.5]undecan-9-ylmethyl)-5-azaspiro[2.4]heptan-5-yl]quinoxaline-5-carbonitrile;
8-[(3S,4R)-3-(2,8-Diazaspiro[4.5]decan-8-ylmethyl)-4-(trifluoromethyl)pyrrolidin-1-yl]quinoxaline-5-carbonitrile;
5-[Trans-3-(3,9-diazaspiro[5.5]undecan-3-ylmethyl)-4-ethyl-pyrrolidin-1-yl]quinoline-8-carbonitrile;
8-[7-(3,9-Diazaspiro[5.5]undecan-3-ylmethyl)-5-azaspiro[2.4]heptan-5-yl]quinoxaline-5-carbonitrile;
8-[(3S,4R)-3-(3,9-diazaspiro[5.5]undecan-3-ylmethyl)-4-methyl-pyrrolidin-1-yl]quinoxaline-5-carbonitrile;
8-[7-[[(3aR,6aS)-2,3,3a,4,6,6a-hexahydro-1H-pyrrolo[3,4-c]pyrrol-5-yl]methyl]-5-azaspiro[2.4]heptan-5-yl]quinoxaline-5-carbonitrile;
5-[Trans-3-(2,7-diazaspiro[4.4]nonan-2-ylmethyl)-4-(trifluoromethyl)pyrrolidin-1-yl]-8-(trifluoromethyl)quinoxaline;
5-[7-(Piperazin-1-ylmethyl)-5-azaspiro[2.4]heptan-5-yl]-8-(trifluoromethyl)quinoxaline;
3-[[Trans-4-methyl-1-[8-(trifluoromethyl)quinoxalin-5-yl]pyrrolidin-3-yl]methyl]-3,9-diazaspiro[5.5]undecane;
8-[7-(2,9-Diazaspiro[4.5]decan-2-ylmethyl)-5-azaspiro[2.4]heptan-5-yl]quinoxaline-5-carbonitrile;
5-[(3S,4R)-3-(3,9-diazaspiro[5.5]undecan-3-ylmethyl)-4-methyl-pyrrolidin-1-yl]quinoline-8-carbonitrile;
8-[7-(2,8-Diazaspiro[3.5]nonan-2-ylmethyl)-5-azaspiro[2.4]heptan-5-yl]quinoxaline-5-carbonitrile;
8-[7-[(3-Amino-8-azabicyclo[3.2.1]octan-8-yl)methyl]-5-azaspiro[2.4]heptan-5-yl]quinoxaline-5-carbonitrile;
8-[(3S,4R)-3-[(4-Amino-1-piperidyl)methyl]-4-(trifluoromethyl)pyrrolidin-1-yl]quinoxaline-5-carbonitrile;
5-[Trans-3-(3,9-diazaspiro[5.5]undecan-3-ylmethyl)-4-(difluoromethyl)pyrrolidin-1-yl]quinoline-8-carbonitrile;
5-[Trans-3-cyclopropyl-4-(3,9-diazaspiro[5.5]undecan-3-ylmethyl)pyrrolidin-1-yl]quinoline-8-carbonitrile;
5-[Trans-3-(2,7-diazaspiro[4.4]nonan-2-ylmethyl)-4-ethyl-pyrrolidin-1-yl]quinoline-8-carbonitrile;
8-[(3S,4R)-3-(2,7-diazaspiro[4.4]nonan-2-ylmethyl)-4-methyl-pyrrolidin-1-yl]quinoxaline-5-carbonitrile;
3-[[5-[8-(Trifluoromethyl)quinoxalin-5-yl]-5-azaspiro[2.4]heptan-7-yl]methyl]-3,9-diazaspiro[5.5]undecane;
8-[7-(2,8-Diazaspiro[4.5]decan-2-ylmethyl)-5-azaspiro[2.4]heptan-5-yl]quinoxaline-5-carbonitrile;
5-[Trans-3-methyl-4-[[4-(1-piperidyl)-1-piperidyl]methyl]pyrrolidin-1-yl]-8-(trifluoromethyl)quinoxaline;
5-[Trans-3-(2,7-diazaspiro[4.4]nonan-2-ylmethyl)-4-(difluoromethyl)pyrrolidin-1-yl]quinoline-8-carbonitrile;
5-[(3S,4R)-3-(2,7-diazaspiro[4.4]nonan-2-ylmethyl)-4-methyl-pyrrolidin-1-yl]quinoline-8-carbonitrile;
5-[Trans-3-(3,9-diazaspiro[5.5]undecan-3-ylmethyl)-4-isopropyl-pyrrolidin-1-yl]quinoline-8-carbonitrile;
8-[3-(3,9-Diazaspiro[5.5]undecan-3-ylmethyl)pyrrolidin-1-yl]quinoxaline-5-carbonitrile;
5-[7-(3,9-Diazaspiro[5.5]undecan-3-ylmethyl)-5-azaspiro[2.4]heptan-5-yl]quinoline-8-carbonitrile;
8-[7-[(Azepan-4-ylamino)methyl]-5-azaspiro[2.4]heptan-5-yl]quinoxaline-5-carbonitrile;
N-[Trans-4-methyl-1-[8-(trifluoromethyl)quinoxalin-5-yl]pyrrolidin-3-yl]methyl]azepan-4-amine;
5-[7-[[(3aR,6aS)-2,3,3a,4,6,6a-hexahydro-1H-pyrrolo[3,4-c]pyrrol-5-yl]methyl]-5-azaspiro[2.4]heptan-5-yl]-8-(trifluoromethyl)quinoxaline;
8-[7-[(6-Amino-2-azaspiro[3.3]heptan-2-yl)methyl]-5-azaspiro[2.4]heptan-5-yl]quinoxaline-5-carbonitrile;
5-[7-(2,7-Diazaspiro[4.4]nonan-2-ylmethyl)-5-azaspiro[2.4]heptan-5-yl]quinoline-8-carbonitrile;
5-[Trans-3-(2,7-diazaspiro[4.4]nonan-2-ylmethyl)-4-isopropyl-pyrrolidin-1-yl]quinoline-8-carbonitrile;
8-[(3S,4R)-3-(2,9-diazaspiro[5.5]undecan-9-ylmethyl)-4-(trifluoromethyl)pyrrolidin-1-yl]quinoxaline-5-carbonitrile;
8-[(3S,4R)-3-(2,8-diazaspiro[4.5]decan-2-ylmethyl)-4-(trifluoromethyl)pyrrolidin-1-yl]quinoxaline-5-carbonitrile;
8-[(3S,4R)-3-[(6-amino-2-azaspiro[3.3]heptan-2-yl)methyl]-4-(trifluoromethyl)pyrrolidin-1-yl]quinoxaline-5-carbonitrile;
8-[(3S,4R)-3-(2,7-diazaspiro[3.5]nonan-2-ylmethyl)-4-(trifluoromethyl)pyrrolidin-1-yl]quinoxaline-5-carbonitrile;
8-[7-(1-Oxa-4,9-diazaspiro[5.5]undecan-9-ylmethyl)-5-azaspiro[2.4]heptan-5-yl]quinoxaline-5-carbonitrile;
8-[7-(3,8-Diazabicyclo[4.2.0]octan-8-ylmethyl)-5-azaspiro[2.4]heptan-5-yl]quinoxaline-5-carbonitrile;
8-[(3S,4R)-3-[(4-amino-1-piperidyl)methyl]-4-(trifluoromethyl)pyrrolidin-1-yl]quinoxaline-5-carbonitrile;
5-[(3S,4R)-3-[(4-piperazin-1-yl-1-piperidyl)methyl]-4-(trifluoromethyl)pyrrolidin-1-yl]quinoline-8-carbonitrile;
5-[(3S,4R)-3-[(6-Amino-2-azaspiro[3.3]heptan-2-yl)methyl]-4-(trifluoromethyl)pyrrolidin-1-yl]quinoline-8-carbonitrile;
5-[(3S,4R)-3-(2,8-Diazaspiro[4.5]decan-8-ylmethyl)-4-(trifluoromethyl)pyrrolidin-1-yl]quinoline-8-carbonitrile;
5-[(3S,4R)-3-(2,9-Diazaspiro[5.5]undecan-9-ylmethyl)-4-(trifluoromethyl)pyrrolidin-1-yl]quinoline-8-carbonitrile;
5-[(3S,4R)-3-(2,8-Diazaspiro[4.5]decan-2-ylmethyl)-4-(trifluoromethyl)pyrrolidin-1-yl]quinoline-8-carbonitrile;
5-[(3S,4R)-3-(2,9-Diazaspiro[5.5]undecan-2-ylmethyl)-4-(trifluoromethyl)pyrrolidin-1-yl]quinoline-8-carbonitrile;
8-[7-[(4-Morpholino-1-piperidyl)methyl]-5-azaspiro[2.4]heptan-5-yl]quinoxaline-5-carbonitrile;
8-[(3S,4R)-3-(Piperazin-1-ylmethyl)-4-(trifluoromethyl)pyrrolidin-1-yl]quinoxaline-5-carbonitrile;
8-[(3S,4R)-3-[(4-Pyrrolidin-1-yl-1-piperidyl)methyl]-4-(trifluoromethyl)pyrrolidin-1-yl]quinoxaline-5-carbonitrile;
8-[(3S,4R)-3-[[4-(1-Piperidyl)-1-piperidyl]methyl]-4-(trifluoromethyl)pyrrolidin-1-yl]quinoxaline-5-carbonitrile;
8-[(3S,4R)-3-[[4-(Azepan-1-yl)-1-piperidyl]methyl]-4-(trifluoromethyl)pyrrolidin-1-yl]quinoxaline-5-carbonitrile;
8-[(3S,4R)-3-[(3-amino-8-azabicyclo[3.2.1]octan-8-yl)methyl]-4-(trifluoromethyl)pyrrolidin-1-yl]quinoxaline-5-carbonitrile;
8-[(3S,4R)-3-[(4-amino-3,3-difluoro-1-piperidyl)methyl]-4-(trifluoromethyl)pyrrolidin-1-yl]quinoxaline-5-carbonitrile;
8-[7-(1,9-Diazaspiro[5.5]undecan-9-ylmethyl)-5-azaspiro[2.4]heptan-5-yl]quinoxaline-5-carbonitrile;
8-[7-(3,7-Diazabicyclo[4.2.0]octan-3-ylmethyl)-5-azaspiro[2.4]heptan-5-yl]quinoxaline-5-carbonitrile;
8-[7-(1-Oxa-4,9-diazaspiro[5.5]undecan-4-ylmethyl)-5-azaspiro[2.4]heptan-5-yl]quinoxaline-5-carbonitrile;
8-[7-[(4-Amino-1-piperidyl)methyl]-5-azaspiro[2.4]heptan-5-yl]quinoxaline-5-carbonitrile;
1-[[(3S,4R)-1-(8-cyanoquinoxalin-5-yl)-4-(trifluoromethyl)pyrrolidin-3-yl]methyl]piperidine-3-carboxamide;
8-[(3S,4R)-3-(1-oxa-4,9-diazaspiro[5.5]undecan-9-ylmethyl)-4-(trifluoromethyl)pyrrolidin-1-yl]quinoxaline-5-carbonitrile;
8-[(3S,4R)-3-[(4-piperidylamino)methyl]-4-(trifluoromethyl)pyrrolidin-1-yl]quinoxaline-5-carbonitrile;
8-[(3S,4R)-3-[[(3S,4R)-4-amino-3-methyl-1-piperidyl]methyl]-4-(trifluoromethyl)pyrrolidin-1-yl]quinoxaline-5-carbonitrile;
8-[(3S,4R)-3-[(8-amino-3-azabicyclo[3.2.1]octan-3-yl)methyl]-4-(trifluoromethyl)pyrrolidin-1-yl]quinoxaline-5-carbonitrile;
8-[(3S,4R)-3-[[4-(aminomethyl)-1-piperidyl]methyl]-4-(trifluoromethyl)pyrrolidin-1-yl]quinoxaline-5-carbonitrile;
8-[(3S,4R)-3-[[3-(aminomethyl)-3-methyl-1-piperidyl]methyl]-4-(trifluoromethyl)pyrrolidin-1-yl]quinoxaline-5-carbonitrile;
8-[(3S,4R)-3-(2,3,3a,4,6,6a-hexahydro-1H-pyrrolo[3,4-c]pyrrol-5-ylmethyl)-4-(trifluoromethyl)pyrrolidin-1-yl]quinoxaline-5-carbonitrile;
8-[(3S,4R)-3-(2,5-diazabicyclo[2.2.2]octan-2-ylmethyl)-4-(trifluoromethyl)pyrrolidin-1-yl]quinoxaline-5-carbonitrile;
8-[(3S,4R)-3-[[3-(aminomethyl)-3-fluoro-1-piperidyl]methyl]-4-(trifluoromethyl)pyrrolidin-1-yl]quinoxaline-5-carbonitrile;
8-[(3S,4R)-3-[[4-(2-aminoethyl)-1-piperidyl]methyl]-4-(trifluoromethyl)pyrrolidin-1-yl]quinoxaline-5-carbonitrile;
8-[(3S,4R)-3-[[4-(dimethylamino)-1-piperidyl]methyl]-4-(trifluoromethyl)pyrrolidin-1-yl]quinoxaline-5-carbonitrile;
8-[(3S,4R)-3-[[4-(methylaminomethyl)-1-piperidyl]methyl]-4-(trifluoromethyl)pyrrolidin-1-yl]quinoxaline-5-carbonitrile;
8-[(3S,4R)-3-[[(3S)-3-[(dimethylamino)methyl]pyrrolidin-1-yl]methyl]-4-(trifluoromethyl)pyrrolidin-1-yl]quinoxaline-5-carbonitrile;
8-[(3S,4R)-3-[[(3R)-3-(dimethylamino)pyrrolidin-1-yl]methyl]-4-(trifluoromethyl)pyrrolidin-1-yl]quinoxaline-5-carbonitrile;
8-[(3S,4R)-3-[(7-methyl-2,7-diazaspiro[4.4]nonan-2-yl)methyl]-4-(trifluoromethyl)pyrrolidin-1-yl]quinoxaline-5-carbonitrile; and
8-[(3S,4R)-3-[[3-(aminomethyl)-3-methyl-pyrrolidin-1-yl]methyl]-4-(trifluoromethyl)pyrrolidin-1-yl]quinoxaline-5-carbonitrile;

or a pharmaceutically acceptable salt, enantiomer or diastereomer thereof.

### Synthesis

The compounds of the present invention can be prepared by any conventional means. Suitable processes for synthesizing these compounds as well as their starting materials are provided in the schemes below and in the examples. All substituents, in particular, R¹ to R⁸ are as defined above unless otherwise indicated. Furthermore, and unless explicitly otherwise stated, all reactions, reaction conditions, abbreviations and symbols have the meanings well known to a person of ordinary skill in organic chemistry.

A general synthetic route for preparing the compound of formula (I), (Ia) or (II) is shown in Scheme 1 below.

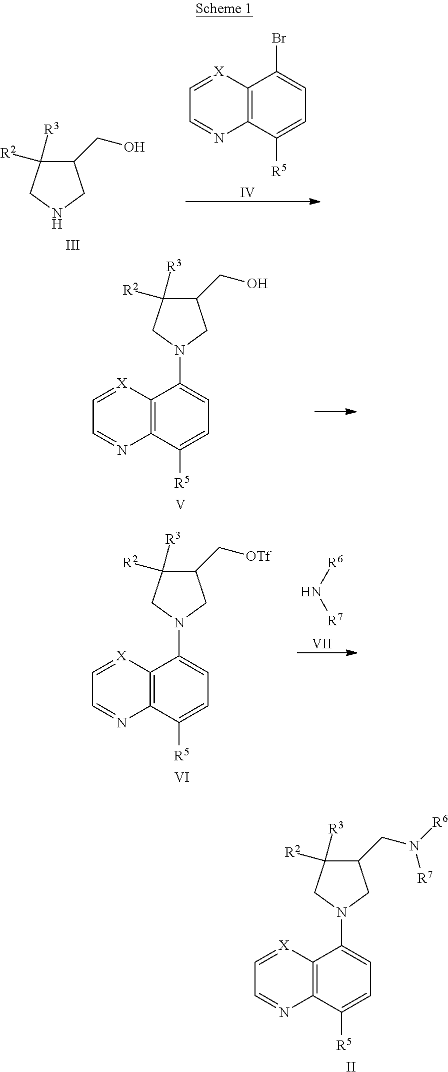

wherein R⁶ and R⁷ are independently selected from H and heterocyclyl, or R⁶ and R⁷ together with the nitrogen they are attached to form a heterocyclyl.

The coupling of halide (IV) with compound of formula (III) can be achieved by direct coupling in the presence of a base, such as DIPEA or K₂CO₃, or under Buchwald-Hartwig amination conditions (ref: Acc. Chem. Res. 1998, 31, 805-818; Chem. Rev. 2016,116, 12564-12649; Topics in Current Chemistry, 2002, 219, 131-209; and references cited therein) with a catalyst, such as Ruphos Pd-G2, and a base, such as Cs₂CO₃, to provide compound of formula (V). Trifluoromethanesulfonation of compound of formula (V) in basic condition, such as 2,6-dimethylpyridine in DCM, gives compound of formula (VI), which is reacted with an amine (VII) in the presence of a base, such as Cs₂CO₃, to give the compound of formula (II). In some embodiment, the reaction of compound of formula (VI) and amine (VII) may give a product containing a protecting group, e.g. Boc, originated from amine (VII), which will be removed before affording the final compound of formula (II).

A compound of formula (I) or (II) when manufactured according to the above process is also an object of the invention.

This invention also relates to a process for the preparation of a compound of formula (I) or (II) comprising any of the following steps:

a) reaction of compound of formula (VI),

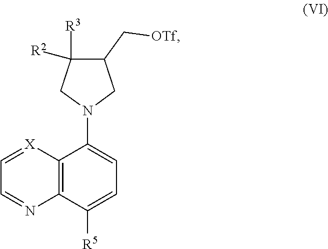

with amine (VII) in the presence of a base;
wherein R², R³, R⁵ and X are defined above.

In step a), the base can be for example Cs₂CO₃.

A compound of formula (I), (Ia) or (II) when manufactured according to the above process is also an object of the invention.

Compounds of this invention can be obtained as mixtures of diastereomers or enantiomers, which can be separated by methods well known in the art, e.g. (chiral) HPLC or SFC.

### Indications and Methods of Treatment

The present invention provides compounds that can be used as TLR7 and/or TLR8 and/or TLR9 antagonist, which inhibits pathway activation through TLR7 and/or TLR8 and/or TLR9 as well as respective downstream biological events including, but not limited to, innate and adaptive immune responses mediated through the production of all types of cytokines and all forms of auto-antibodies. Accordingly, the compounds of the invention are useful for blocking TLR7 and/or TLR8 and/or TLR9 in all types of cells that express such receptor(s) including, but not limited to, plasmacytoid dendritic cell, B cell, T cell, macrophage, monocyte, neutrophil, keratinocyte, epithelial cell. As such, the compounds can be used as a therapeutic or prophylactic agent for systemic lupus erythematosus and lupus nephritis.

The present invention provides methods for treatment or prophylaxis of systemic lupus erythematosus and lupus nephritis in a patient in need thereof.

Another embodiment includes a method of treating or preventing systemic lupus erythematosus and lupus nephritis in a mammal in need of such treatment, wherein the method comprises administering to said mammal a therapeutically effective amount of a compound of formula (I), a stereoisomer, tautomer, prodrug or pharmaceutically acceptable salt thereof.

### EXAMPLES

The invention will be more fully understood by reference to the following examples. They should not, however, be construed as limiting the scope of the invention.

### ABBREVIATIONS

The invention will be more fully understood by reference to the following examples. They should not, however, be construed as limiting the scope of the invention.

Abbreviations used herein are as follows:
ACN: acetonitrile
Boc₂O: di-tert-butyl dicarbonate
Tf₂O: triflic anhydride
DCM: dichloromethane
DIPEA diethylisopropylamine
EA or EtOAc: ethyl acetate
FA: formic acid
HATU 1-[Bis(dimethylamino)methylene]-1H-1,2,3-triazolo[4,5-b]pyridinium 3-oxid hexafluorophosphate
HLM human liver micro some
IC₅₀: half inhibition concentration
LCMS liquid chromatography-mass spectrometry
LYSA lyophilisation solubility assay
MS: mass spectrometry
PE: petroleum ether
prep-HPLC: preparative high performance liquid chromatography
rt: room temperature
RT: retention time
RuPhos Pd G2: chloro(2-dicyclohexylphosphino-2′,6′-diisopropoxy-1,1′-biphenyl)[2-(2′-amino-1,1′-biphenyl)]palladium(II) 2nd generation
SFC: supercritical fluid chromatography
Tf: trifluoromethanesulfonyl
TFA: trifluoroacetic acid
v/v volume ratio

General Experimental Conditions

Intermediates and final compounds were purified by flash chromatography using one of the following instruments: i) Biotage SP1 system and the Quad 12/25 Cartridge module, ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃.H₂O in MeOH), back pressure 100bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):

Acidic condition I: A: 0.1% TFA in H₂O; B: 0.1% TFA in acetonitrile;

Acidic condition II: A: 0.0375% TFA in H₂O; B: 0.01875% TFA in acetonitrile;

Basic condition I: A: 0.1% NH₃H₂O in H₂O; B: acetonitrile;

Basic condition II: A: 0.025% NH₃H₂O in H₂O; B: acetonitrile;

Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Example 1

### 8-[(3S,4R)-3-(3,9-diazaspiro[5.5]undecan-3-ylmethyl)-4-(trifluoromethyl)pyrrolidin-1-yl]quinoxaline-5-carbonitrile

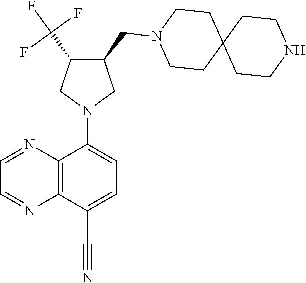 [SMILES: N#Cc1ccc(N2C[C@H](CN3CCC4(CCNCC4)CC3)[C@@H](C(F)(F)F)C2)c2nccnc12]

The title compound was prepared according to the following scheme:

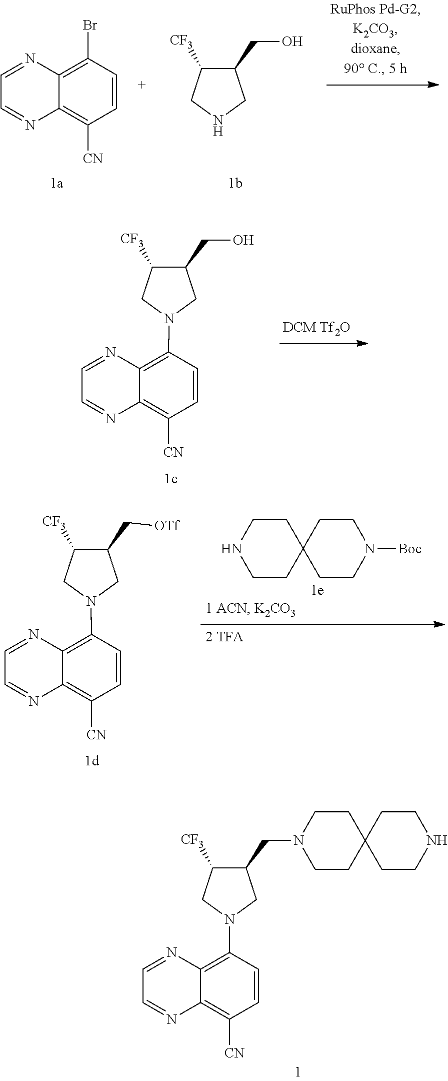 [SMILES: N#Cc1ccc(Br)c2nccnc12 ; OC[C@H]1CNC[C@@H]1C(F)(F)F ; N#Cc1ccc(N2C[C@H](CO)[C@@H](C(F)(F)F)C2)c2nccnc12 ; N#Cc1ccc(N2C[C@H](COS(=O)(=O)C(F)(F)F)[C@@H](C(F)(F)F)C2)c2nccnc12 ; CC(C)(C)OC(=O)N1CCC2(CCNCC2)CC1 ; N#Cc1ccc(N2C[C@H](CN3CCC4(CCNCC4)CC3)[C@@H](C(F)(F)F)C2)c2nccnc12]

### Step 1: preparation of 8-[(3R,4R)-3-(hydroxymethyl)-4-(trifluoromethyl)pyrrolidin-1-yl]quinoxaline-5-carbonitrile (Compound 1c)

To a solution of ((3R,4R)-4-(trifluoromethyl)pyrrolidin-3-yl)methanol hydrochloride salt (compound 1b, 57 mg, 278 μmol, Pharmablock, PBXA3261-1) and 8-bromoquinoxaline-5-carbonitrile (compound 1a, 50 mg, 214 μmol) (Reference: WO2017/106607) in 1,4-dioxane (10 mL) was added K₂CO₃ (148 mg, 1.07 mmol). The mixture was degassed three times, then Ruphos Pd G2 (CAS: 1375325-68-0, 16 mg, 21.4 μmol) was added. The reaction mixture was stirred at 90° C. for 5 hrs under N₂, then cooled to rt, diluted with EA (50 mL) and washed with water. The organic layer was concentrated to afford a crude product which was purified by silica gel column chromatography eluting with a gradient of PE:EA (from 0 to 70%) to give 8-[(3R,4R)-3-(hydroxymethyl)-4-(trifluoromethyl)pyrrolidin-1-yl]quinoxaline-5-carbonitrile (compound 1c, 48 mg). MS: calc'd 323 (MH⁺), measured 323 (MH⁺).

### Step 2: Preparation of ((3R,4R)-1-(8-cyanoquinoxalin-5-yl)-4-(trifluoromethyl)pyrrolidin-3-yl)methyl trifluoromethanesulfonate (Compound 1d)

To a solution of 8-((3R,4R)-3-(hydroxymethyl)-4-(trifluoromethyl)pyrrolidin-1-yl)quinoxaline-5-carbonitrile (compound 1c, 48 mg, 149 μmol) in DCM (20 mL) was added 2,6-dimethylpyridine (31 mg, 298 μmol). A yellow solution was formed, then it was cooled with ice bath. Trifluoromethanesulfonic anhydride (63 mg, 223 μmol) was added drop-wise into the mixture, which was kept in the ice bath for 1 hour. Then the mixture was diluted with 30 mL DCM and washed with sat. NH₄Cl (30 mL) twice. The organic layer was dried over Na₂SO₄ and concentrated to give a brown solid, which was purified by silica gel column chromatography eluting with a gradient of PE:EA (from 0 to 70%) to give ((3R,4R) 1-(8-cyanoquinoxalin-5-yl)-4-(trifluoromethyl)pyrrolidin-3-yl)methyl trifluoromethanesulfonate (compound 1d, 67 mg). MS: calc'd 455 (MH⁺), measured 455 (MH⁺).

### Step 3: 8-[(3S,4R)-3-(3,9-diazaspiro[5.5]undecan-3-ylmethyl)-4-(trifluoromethyl)pyrrolidin-1-yl]quinoxaline-5-carbonitrile (Example 1)

To a solution of ((3R,4R) 1-(8-cyanoquinoxalin-5-yl)-4-(trifluoromethyl)pyrrolidin-3-yl)methyl trifluoromethanesulfonate (compound 1d, 30 mg, 66 μmol) and tert-butyl 3,9-diazaspiro[5.5]undecane-3-carboxylate (compound 1e, 16 mg, 66 μmol, Bide, CAS: 173405-78-2) in acetonitrile (4 mL) was added K₂CO₃ (36 mg, 264 μmol). After the mixture was heated to reflux for 4 hours, it was diluted with ACN and filtered through celite. The filtrate was concentrated to give a yellow intermediate. The intermediate was dissolved in 5 mL DCM, to which 0.5 mL TFA was added. After the reaction mixture was stirred for 2 hours at r.t, it was concentrated to afford an oil, which was purified by prep-HPLC to give Example 1 (7 mg) as a yellow solid. MS: calc'd 459 (MH⁺), measured 459 (MH⁺). ¹H NMR (400 MHz, METHANOL-d₄) δ=8.79 (d, J=1.7 Hz, 1H), 8.72 (d, J=1.8 Hz, 1H), 7.93 (d, J=8.6 Hz, 1H), 6.77 (d, J=8.6 Hz, 1H), 4.37-4.24 (m, 2H), 4.22-4.08 (m, 1H), 3.84 (dd, J=6.8, 11.2 Hz, 1H), 3.47-3.30 (m, 4H), 3.15-2.90 (m, 8H), 2.03-1.53 (m, 8H).

### Example 2

### 5-[(3S,4R)-3-(3,9-diazaspiro[5.5]undecan-3-ylmethyl)-4-(trifluoromethyl)pyrrolidin-1-yl]quinoline-8-carbonitrile

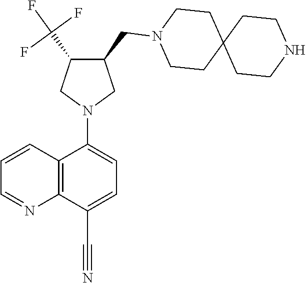 [SMILES: N#Cc1ccc(N2C[C@H](CN3CCC4(CCNCC4)CC3)[C@@H](C(F)(F)F)C2)c2cccnc12]

The title compound was prepared in analogy to the preparation of Example 1 by using 5-bromoquinoline-8-carbonitrile instead of bromoquinoxaline-5-carbonitrile (compound 1a). Example 2 (10 mg) was obtained as a yellow solid. MS: calc'd 458 (MH⁺), measured 458 (MH⁺). ¹H NMR (400 MHz, METHANOL-d₄) δ=8.85 (dd, J=1.6, 4.3 Hz, 1H), 8.59 (dd, J=1.6, 8.7 Hz, 1H), 7.97 (d, J=8.2 Hz, 1H), 7.49 (dd, J=4.3, 8.7 Hz, 1H), 6.99 (d, J=8.3 Hz, 1H), 3.89 (dd, J=7.0, 9.8 Hz, 1H), 3.78-3.68 (m, 1H), 3.63 (dd, J=6.2, 10.6 Hz, 2H), 3.50-3.35 (m, 4H), 3.16-3.00 (m, 8H), 2.00-1.46 (m, 8H).

### Example 3

### 8-[7-(piperazin-1-ylmethyl)-5-azaspiro[2.4]heptan-5-yl]quinoxaline-5-carbonitrile

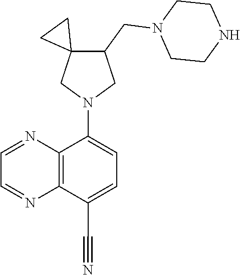 [SMILES: N#Cc1ccc(N2CC(CN3CCNCC3)C3(CC3)C2)c2nccnc12]

The title compound was prepared according to the following scheme:

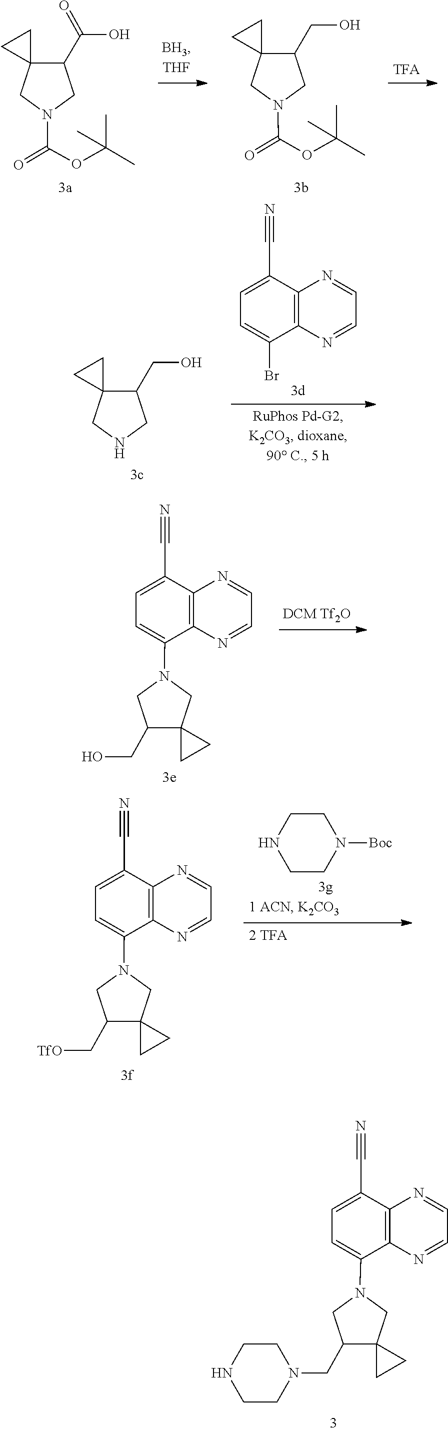

### Step 1: preparation of tert-butyl 7-(hydroxymethyl)-5-azaspiro[2.4]heptane-5-carboxylate

To a solution of 5-(tert-butoxycarbonyl)-5-azaspiro[2.4]heptane-7-carboxylic acid (compound 3a, 2 g, 8.29 mmol, Pharmablock, PBLJ7032) in THF (50 mL) was added borane tetrahydrofuran complex (41 mL, 41.4 mmol,) at 0° C. The mixture was stirred for 6 hrs at 25° C. The reaction mixture was poured into saturated aqueous NaHCO₃ and extracted with CH₂Cl₂. The organic layer was washed with brine, dried over Na₂SO₄ and concentrated in vacuo to give the crude product (1.5 g) which was used in the next step without purification. MS: calc'd 228 (MH⁺), measured 228 (MH⁺).

### Step 2: preparation of 5-azaspiro[2.4]heptan-7-ylmethanol

To a solution of tert-butyl 7-(hydroxymethyl)-5-azaspiro[2.4]heptane-5-carboxylate (compound 3b, 1.5 g, 6.6 mmol, crude) in DCM (5 mL) was added 2,2,2-trifluoroacetic acid (5.27 g, 3.43 mL, 46.2 mmol). The reaction mixture was stirred at r.t. for 3 hours. Then the reaction mixture was concentrated in vacuo to give the crude product (750 mg) which was used in the next step without purification. MS: calc'd 128 (MH⁺), measured 128 (MH⁺).

### Step 3: preparation of 8-(7-(hydroxymethyl)-5-azaspiro[2.4]heptan-5-yl)quinoxaline-5-carbonitrile

To a solution of 8-bromoquinoxaline-5-carbonitrile (compound 1a, 400 mg, 1.71 mmol) and 5-azaspiro[2.4]heptan-7-ylmethanol (compound 3c, 378 mg, 2.97 mmol) in 1,4-dioxane (20 mL) was added cesium carbonate (2.23 g, 6.84 mmol). The mixture was degassed three times, then Ruphos Pd G2 (92.9 mg, 120 μmol) was added. The reaction mixture was stirred at 90° C. for 5 hours under N₂. The mixture was cooled to r.t., diluted with EA (50 mL) and washed with water. Then the organic layer was concentrated to afford crude product which was purified to get compound 3e (370 mg) as a dark brown solid by combi-flash with PE: EA (from 0 to 50%). MS: calc'd 281 (MH⁺), measured 281 (MH⁺).

### Step 4: preparation of (5-(8-cyanoquinoxalin-5-yl)-5-azaspiro[2.4]heptan-7-yl)methyl trifluoromethanesulfonate

To a solution of 8-(7-(hydroxymethyl)-5-azaspiro[2.4]heptan-5-yl)quinoxaline-5-carbonitrile (compound 3e, 370 mg, 1.32 mmol) in DCM (40 mL) was added 2,6-dimethylpyridine (283 mg, 307 μL, 2.64 mmol). The reaction mixture was cooled with ice bath and trifluoromethanesulfonic anhydride (559 mg, 325 μL, 1.98 mmol) was added drop-wise. After the mixture was kept in the ice bath for 1 hour, it was diluted with 30 mL DCM and washed with sat. NH₄Cl (30 mL) twice. The organic layer was dried over Na₂SO₄ and concentrated to give the crude product (500 mg) which was used in the next step without purification. MS: calc'd 413 (MH⁺), measured 413 (MH⁺).

### Step 5: preparation of 8-[7-(piperazin-1-ylmethyl)-5-azaspiro[2.4]heptan-5-yl]quinoxaline-5-carbonitrile

To a solution of (5-(8-cyanoquinoxalin-5-yl)-5-azaspiro[2.4]heptan-7-yl)methyl trifluoromethanesulfonate (compound 3f, 50 mg, 121 μmol), tert-butyl piperazine-1-carboxylate (compound 3g, 34 mg, 182 μmol) in acetonitrile (6 mL) was added K₂CO₃ (34 mg, 242 μmol). The mixture was heated to reflux for 4 hours, then diluted with ACN and filtered through celite. The filtrate was concentrated to give a light brown intermediate. The intermediate was dissolved in 5 mL DCM. Then 0.5 mL TFA was added to the solution. After the mixture was stirred for 3 hours at r.t., it was concentrated to afford an oil, which was purified by prep-HPLC to give Example 3 (36 mg) as a light yellow solid. MS: calc'd 349 (MH⁺), measured 349 (MH⁺). ¹H NMR (400 MHz, DMSO-d6) δ 8.95 (d, J=1.71 Hz, 1H), 8.82 (d, J=1.83 Hz, 1H), 8.07 (d, J=8.68 Hz, 1H), 6.74 (d, J=8.80 Hz, 1H), 3.97-4.17 (m, 2H), 3.88 (d, J=8.80 Hz, 1H), 3.75 (d, J=12.35 Hz, 1H), 3.14 (m, 5H), 2.57-2.90 (m, 5H), 2.21-2.36 (m, 1H), 0.76-0.90 (m, 1H), 0.55-0.73 (m, 3H).

### Example 4

### 8-[7-[(8-Amino-3-azabicyclo[3.2.1]octan-3-yl)methyl]-5-azaspiro[2.4]heptan-5-yl]quinoxaline-5-carbonitrile

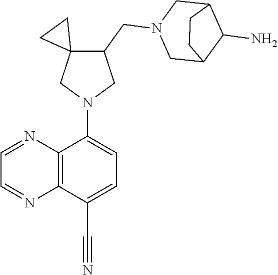 [SMILES: N#Cc1ccc(N2CC(CN3CC4CCC(C3)C4N)C3(CC3)C2)c2nccnc12]

The title compound was prepared in analogy to the preparation of Example 3 by using tert-butyl N-(3-azabicyclo[3.2.1]octan-8-yl)carbamate instead of tert-butyl piperazine-1-carboxylate (compound 3g). Example 4 (18 mg) was obtained as a yellow solid. MS: calc'd 389 (MH⁺), measured 389 (MH⁺). ¹H NMR (400 MHz, METHANOL-d₄) δ=8.81-8.89 (m, 1H), 8.73-8.79 (m, 1H), 7.92-8.02 (m, 1H), 6.77 (t, J=7.64 Hz, 1H), 4.29-4.40 (m, 1H), 4.21 (d, J=10.52 Hz, 1H), 4.11 (d, J=11.74 Hz, 1H), 3.74 (d, 7=11.49 Hz, 1H), 3.41-3.59 (m, 3H), 3.13-3.29 (m, 4H), 2.42-2.62 (m, 3H), 1.85-2.16 (m, 4H), 0.74-0.97 (m, 4H).

### Example 5

### 8-[(3S,4R)-3-(2,7-diazaspiro[4.4]nonan-2-ylmethyl)-4-(trifluoromethyl)pyrrolidin-1-yl]quinoxaline-5-carbonitrile

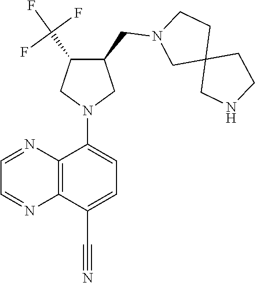 [SMILES: N#Cc1ccc(N2C[C@H](CN3CCC4(CCNC4)C3)[C@@H](C(F)(F)F)C2)c2nccnc12]

The title compound was prepared in analogy to the preparation of Example 1 by using tert-butyl 2,7-diazaspiro[4.4]nonane-2-carboxylate instead of tert-butyl 3,9-diazaspiro[5.5]undecane-3-carboxylate (compound 1e). Example 5 (5 mg) was obtained as a yellow solid. MS: calc'd 431 (MH⁺), measured 431 (MH⁺). ¹H NMR (400 MHz, METHANOL-d₄) δ=8.88 (d, 7=1.7 Hz, 1H), 8.81 (d, 7=1.7 Hz, 1H), 8.01 (d, 7=8.6 Hz, 1H), 6.86 (d, 7=8.4 Hz, 1H), 4.45-4.23 (m, 3H), 3.94 (dd, 7=6.8, 11.5 Hz, 1H), 3.57 (br, 6H), 3.49-3.37 (m, 5H), 3.16-3.03 (m, 1H), 2.35-2.11 (m, 4H).

### Example 6

### 5-[(3S,7R)-3-(2,9-diazaspiro[4.5]decan-2-ylmethyl)-4-(trifluoromethyl)pyrrolidin-1-yl]quinoline-8-carbonitrile

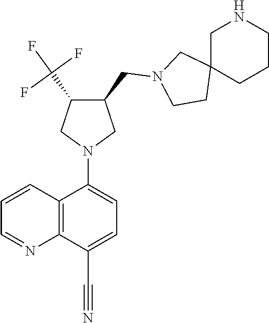 [SMILES: N#Cc1ccc(N2C[C@H](CN3CCC4(CCCNC4)C3)[C@@H](C(F)(F)F)C2)c2cccnc12]

The title compound was prepared in analogy to the preparation of Example 1 by using 5-bromoquinoline-8-carbonitrile and tert-butyl-2,7-diazaspiro[4.5]decane-7-carboxylate (CAS: 236406-61-4) instead of bromoquinoxaline-5-carbonitrile (compound 1a) and tert-butyl 3,9-diazaspiro[5.5]undecane-3-carboxylate (compound 1e). Example 6 (20 mg) was obtained as a yellow solid. MS: calc'd 444 (MH⁺), measured 444 (MH⁺). ¹H NMR (400 MHz, METHANOL-d₄) δ=8.97-8.87 (m, 1H), 8.71 (dd, J=1.5, 8.7 Hz, 1H), 8.05 (d, 7=8.3 Hz, 1H), 7.56 (dd, 7=4.3, 7.8 Hz, 1H), 7.03 (d, 7=8.2 Hz, 1H), 3.89 (ddd, 7=2.8, 8.2, 10.8 Hz, 1H), 3.82-3.71 (m, 1H), 3.72-3.59 (m, 1H), 3.57-3.44 (m, 1H), 3.19-3.01 (m, 5H), 2.90-2.55 (m, 6H), 2.41 (dd, 7 5=9.6, 18.5 Hz, 1H), 1.88-1.60 (m, 6H).

### Example 7

### 8-[7-[(4-Amino-4-methyl-1-piperidyl)methyl]-5-azaspiro[2.4]heptan-5-yl]quinoxaline-5-carbonitrile

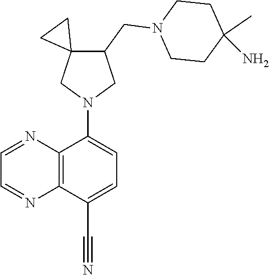 [SMILES: CC1(N)CCN(CC2CN(c3ccc(C#N)c4nccnc34)CC23CC3)CC1]

The title compound was prepared in analogy to the preparation of Example 3 by using tert-butyl N-(4-methyl-4-piperidyl/carbamate instead of tert-butyl piperazine-1-carboxylate (compound 3g). Example 7 (15 mg) was obtained as a yellow solid. MS: calc'd 377 (MH⁺), measured 377 (MH⁺). ¹H NMR (400 MHz, METHANOL-d₄) δ=8.85 (d, 7=1.71 Hz, 1H), 8.76 (d, 7=1.71 Hz, 1H), 7.97 (d, 7=8.68 Hz, 1H), 6.78 (d, 7=8.80 Hz, 1H), 4.33-4.42 (m, 1H), 4.23-4.29 (m, 1H), 4.18 (d, 7=11.74 Hz, 1H), 3.47-3.77 (m, 3H), 3.37-3.45 (m, 1H), 3.23 d, J=11.37 Hz, 2H), 2.42-2.52 (m, 1H), 2.15-2.31 (m, 2H), 2.04-2.15 (m, 2H), 1.53 (s, 3H), 0.74-0.98 (m, 4H).

### Example 8

### 5-[(3S,7R)-3-(2,8-diazaspiro[3.5]nonan-2-ylmethyl)-4-(trifluoromethyl)pyrrolidin-1-yl]quinoline-8-carbonitrile

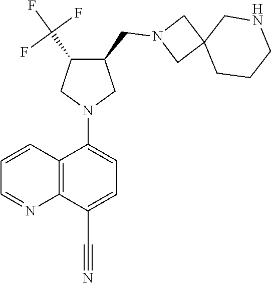 [SMILES: N#Cc1ccc(N2C[C@H](CN3CC4(CCCNC4)C3)[C@@H](C(F)(F)F)C2)c2cccnc12]

The title compound was prepared in analogy to the preparation of Example 1 by using 5-bromoquinoline-8-carbonitrile and tert-butyl-2,6-diazaspiro[3.5]nonane-6-carboxylate (WuXi Apptec, CAS:885272-17-3) instead of bromoquinoxaline-5-carbonitrile (compound 1a) and tert-butyl 3,9-diazaspiro[5.5]undecane-3-carboxylate (compound 1e). Example 8 (3 mg) was obtained as a yellow solid. MS: calc'd 430 (MH⁺), measured 430 (MH⁺). ¹H NMR (400 MHz, METHANOL-d₄) δ=9.01-8.88 (m, 1H), 8.70 (dd, J=1.6, 8.6 Hz, 1H), 8.05 (d, J=8.3 Hz, 1H), 7.56 (dd, J=4.2, 8.7 Hz, 1H), 7.02 (d, J=8.4 Hz, 1H), 3.91-3.62 (m, 4H), 3.54-3.38 (m, 3H), 3.15-2.97 (m, 6H), 2.81-2.53 (m, 3H), 1.95-1.66 (m, 4H).

### Example 9

### 5-[(3S,4R)-3-(2,7-diazaspiro[4.4]nonan-2-ylmethyl)-4-(trifluoromethyl)pyrrolidin-1-yl]quinoline-8-carbonitrile

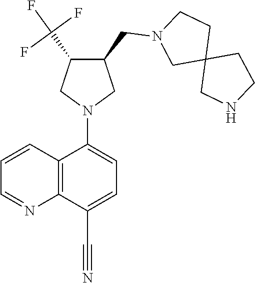 [SMILES: N#Cc1ccc(N2C[C@H](CN3CCC4(CCNC4)C3)[C@@H](C(F)(F)F)C2)c2cccnc12]

The title compound was prepared in analogy to the preparation of Example 1 by using 5-bromoquinoline-8-carbonitrile and tert-butyl 2,7-diazaspiro[4.4]nonane-2-carboxylate instead of bromoquinoxaline-5-carbonitrile (compound 1a) and tert-butyl 3,9-diazaspiro[5.5]undecane-3-carboxylate (compound 1e). Example 9 (5 mg) was obtained as a yellow solid. MS: calc'd 430 (MH⁺), measured 430 (MH⁺). ¹H NMR (400 MHz, METHANOL-d₄) δ=8.85 (dd, J=1.6, 4.3 Hz, 1H), 8.63-8.53 (m, 1H), 7.97 (d, J=8.2 Hz, 1H), 7.49 (dd, J=4.3, 8.7 Hz, 1H), 6.99 (d, J=8.2 Hz, 1H), 3.87 (br t, J=12 Hz, 1H), 3.76-3.68 (m, 1H), 3.66-3.40 (m, 7H), 3.36-3.26 (m, 5H), 3.16-2.91 (m, 2H), 2.24-2.04 (m, 4H).

### Example 10

### 5-[(3S,4R)-3-[[4-(1-piperidyl)-1-piperidyl]methyl]-4-(trifluoromethyl)pyrrolidin-1-yl]quinoline-8-carbonitrile

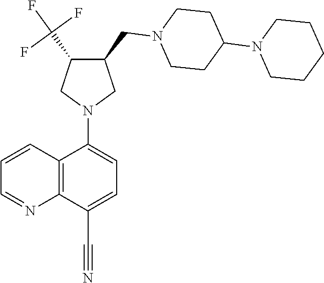 [SMILES: N#Cc1ccc(N2C[C@H](CN3CCC(N4CCCCC4)CC3)[C@@H](C(F)(F)F)C2)c2cccnc12]

The title compound was prepared in analogy to the preparation of Example 1 by using 5-bromoquinoline-8-carbonitrile (Titan, CAS:4897-50-1) and 1-(4-piperidyl)piperidine instead of bromoquinoxaline-5-carbonitrile (compound 1a) and tert-butyl 3,9-diazaspiro[5.5]undecane-3-carboxylate (compound 1e). Example 10 (20 mg) was obtained as a yellow solid. MS: calc'd 472 (MH⁺), measured 472 (MH⁺). ¹H NMR (400 MHz, METHANOL-d₄) δ=8.93 (dd, J=1.5, 4.2 Hz, 1H), 8.67 (dd, J=1.5, 8.7 Hz, 1H), 8.03 (d, J=8.2 Hz, 1H), 7.57 (dd, J=4.3, 8.7 Hz, 1H), 7.05 (d, J=8.2 Hz, 1H), 3.98 (br dd, J=7.1, 9.8 Hz, 2H), 3.89-3.76 (m, 2H), 3.78-3.66 (m, 1H), 3.62-3.40 (m, 6H), 3.28-2.95 (m, 6H), 2.40 (br d, J=11.1 Hz, 2H), 2.21 (q, J=12.5 Hz, 2H), 2.07-1.49 (m, 6H).

### Example 11

### 8-[7-[[4-(1-Piperidyl)-1-piperidyl]methyl]-5-azaspiro[2.4]heptan-5-yl]quinoxaline-5-carbonitrile

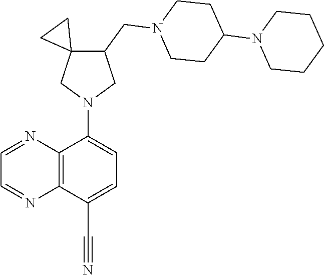 [SMILES: N#Cc1ccc(N2CC(CN3CCC(N4CCCCC4)CC3)C3(CC3)C2)c2nccnc12]

The title compound was prepared in analogy to the preparation of Example 3 by using 1-(4-piperidyl)piperidine instead of tert-butyl piperazine-1-carboxylate (compound 3g). Example 11 (22 mg) was obtained as a yellow solid. MS: calc'd 431 (MH⁺), measured 431 (MH⁺). ¹H NMR (400 MHz, METHANOL-d₄) δ=8.84 (d, J=1.83 Hz, 1H), 8.76 (d, J=1.83 Hz, 1H), 7.98 (d, J=8.68 Hz, 1H), 6.77 (d, J=8.68 Hz, 1H), 4.16-4.25 (m, 1H), 4.01-4.16 (m, 2H), 3.78 (d, J=11.98 Hz, 1H), 2.95-3.29 (m, 7H), 2.53-2.64 (m, 1H), 2.37-2.48 (m, 1H), 2.23-2.34 (m, 2H), 2.05-2.17 (m, 3H), 1.62-1.96 (m, 8H), 0.82-0.93 (m, 1H), 0.70 (s, 3H).

### Example 12

### 8-[(3S,4R)-3-[(4-amino-4-methyl-1-piperidyl)methyl]-4-(trifluoromethyl)pyrrolidin-1-yl]quinoxaline-5-carbonitrile

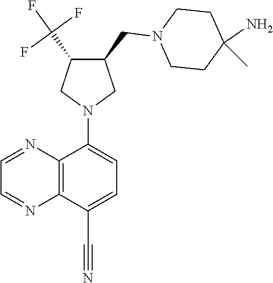 [SMILES: CC1(N)CCN(C[C@H]2CN(c3ccc(C#N)c4nccnc34)C[C@@H]2C(F)(F)F)CC1]

The title compound was prepared in analogy to the preparation of Example 1 by using tert-butyl N-(4-methyl-4-piperidyl/carbamate instead of tert-butyl 3,9-diazaspiro[5.5]undecane-3-carboxylate (compound 1e). Example 12 (15 mg) was obtained as a yellow solid. MS: calc'd 419 (MH⁺), measured 419 (MH⁺). ¹H NMR (400 MHz, METHANOL-d₄) δ=8.86 (d, J=1.7 Hz, 1H), 8.79 (d, J=1.7 Hz, 1H), 7.97 (d, J=8.6 Hz, 1H), 6.81 (d, J=8.6 Hz, 1H), 4.45-4.21 (m, 3H), 3.90 (dd, J=6.5, 11.6 Hz, 1H), 3.74-3.39 (m, 4H), 3.22-3.04 (m, 4H), 2.31-1.99 (m, 4H), 1.62-1.41 (m, 3H).

### Example 13

### 8-[7-(2,9-Diazaspiro[5.5]undecan-9-ylmethyl)-5-azaspiro[2.4]heptan-5-yl]quinoxaline-5-carbonitrile

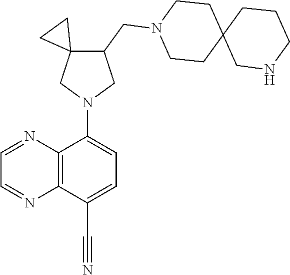 [SMILES: N#Cc1ccc(N2CC(CN3CCC4(CCCNC4)CC3)C3(CC3)C2)c2nccnc12]

The title compound was prepared in analogy to the preparation of Example 3 by using tert-butyl 2,9-diazaspiro[5.5]undecane-2-carboxylate (PharmaBlock, CAS: 189333-03-7) instead of tert-butyl piperazine-1-carboxylate (compound 3g). Example 13 (17 mg) was obtained as a yellow solid. MS: calc'd 417 (MH⁺), measured 417 (MH⁺). ¹H NMR (400 MHz, METHANOL-d₄) δ=8.84 (d, J=1.71 Hz, 1H), 8.74 (d, J=1.71 Hz, 1H), 7.94 (d, J=8.68 Hz, 1H), 6.76 (d, J=8.80 Hz, 1H), 4.32-4.43 (m, 1H), 4.22-4.32 (m, 1H), 4.17 (br d, J=11.62 Hz, 1H), 3.68 (d, J=11.62 Hz, 2H), 3.53 (br d, J=10.39 Hz, 1H), 3.40 (dd, J=10.82, 13.14 Hz, 1H), 3.05-3.26 (m, 5H), 3.00 (s, 1H), 2.42-2.54 (m, 1H), 1.57-2.14 (m, 9H), 0.73-0.97 (m, 4H).

### Example 14

### 8-[(3S,4R)-3-(2,8-Diazaspiro[4.5]decan-8-ylmethyl)-4-(trifluoromethyl)pyrrolidin-1-yl]quinoxaline-5-carbonitrile

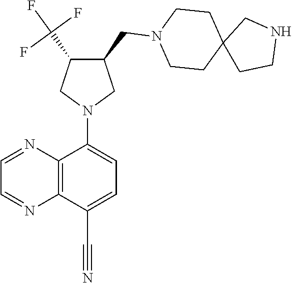 [SMILES: N#Cc1ccc(N2C[C@H](CN3CCC4(CCNC4)CC3)[C@@H](C(F)(F)F)C2)c2nccnc12]

The title compound was prepared in analogy to the preparation of Example 1 by using tert-butyl 2,8-diazaspiro[4.5]decane-2-carboxylate (PharmaBlock, CAS:336191-17-4) instead of tert-butyl 3,9-diazaspiro[5.5]undecane-3-carboxylate (compound 1e). Example 14 (18 mg) was obtained as a yellow solid. MS: calc'd 445 (MH⁺), measured 445 (MH⁺). ¹H NMR (400 MHz, METHANOL-d₄) δ=8.88 (d, J=1.8 Hz, 1H), 8.80 (d, J=1.7 Hz, 1H), 7.99 (d, J=8.6 Hz, 1H), 6.84 (d, J=8.7 Hz, 1H), 4.39 (br dd, J=8.7, 12.0 Hz, 2H), 4.32-4.15 (m, 1H), 3.93 (dd, J=6.7, 11.4 Hz, 1H), 3.55-3.39 (m, 4H), 3.26-3.09 (m, 8H), 2.15-1.85 (m, 6H).

### Example 15

### 5-[Trans-3-(3,9-diazaspiro[5.5]undecan-3-ylmethyl)-4-ethyl-pyrrolidin-1-yl]quinoline-8-carbonitrile

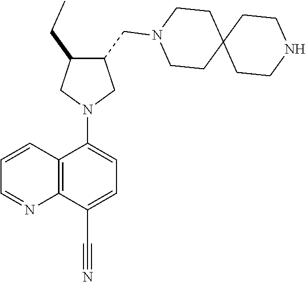 [SMILES: CC[C@@H]1CN(c2ccc(C#N)c3ncccc23)C[C@H]1CN1CCC2(CCNCC2)CC1]

The title compound was prepared according to the following scheme:

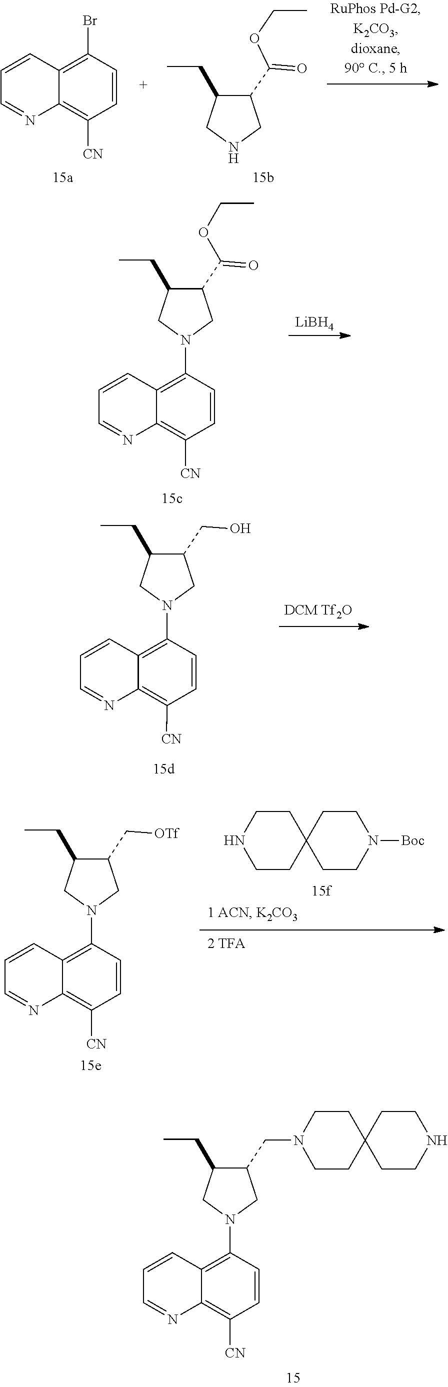

### Step 1: preparation of ethyl trans-1-(8-cyano-5-quinolyl)-4-ethyl-pyrrolidine-3-carboxylate (Compound 15c)

To a solution of ethyl trans-4-ethylpyrrolidine-3-carboxylate hydrochloride salt (compound 15b, 267 mg, 1.29 mmol, Pharmablock, PBXA3209-1) and 5-bromoquinoline-8-carbonitrile (compound 15a, 300 mg, 1.29 mmol) in 1,4-dioxane (10 mL) was added K₂CO₃ (889 mg, 6.44 mmol). The mixture was degassed three times, then Ruphos Pd G2 (100 mg, 129 μmol) was added. After the reaction mixture was stirred at 90° C. for 5 hrs under N₂, it was cooled to rt, diluted with EA (150 mL) and washed with water. Then the organic layer was concentrated to afford a crude product (416 mg) which was used in the next step without purification. MS: calc'd 324 (MH⁺), measured 324 (MH⁺).

### Step 2: preparation of 5-[trans-3-ethyl-4-(hydroxymethyl)pyrrolidin-1-yl]quinoline-8-carbonitrile (Compound 15d)

Lithium tetrahydroborate (67.4 mg, 3.09 mmol) was added to a solution of ethyl trans-1-(8-cyano-5-quinolyl)-4-ethyl-pyrrolidine-3-carboxylate (compound 15c, 100 mg, 309 μmol) in THF (10 mL). After the mixture was stirred at r.t. overnight, it was diluted with DCM (50 mL) and filtered. The solution was concentrated to afford an oil, which was purified by column chromatography to give compound 15d (40 mg). MS: calc'd 282 (MH⁺), measured 282 (MH⁺).

### Step 3: preparation of [trans-1-(8-cyano-5-quinolyl)-4-ethyl-pyrrolidin-3-yl]methyl trifluoromethanesulfonate (Compound 15e)

To a solution of 5-[trans-3-ethyl-4-(hydroxymethyl)pyrrolidin-1-yl]quinoline-8-carbonitrile (compound 15d, 40 mg, 142 μmol) in DCM (20 mL) was added 2,6-dimethylpyridine (30 mg, 284 μmol). A yellow solution was formed, then it was cooled with ice bath. And trifluoromethanesulfonic anhydride (60 mg, 213 μmol) was added drop-wise into the mixture. After the mixture was kept in the ice bath for 1 hour, it was diluted with 30 mL DCM and washed with sat. NH₄Cl (30 mL) twice. The organic layer was dried over Na₂SO₄ and concentrated to give a brown solid, which was purified by column chromatography (EA/PE=0˜30%) to give compound 1e (50 mg). MS: calc'd 414 (MH⁺), measured 414 (MH⁺).

### Step 4: 5-[trans-3-(3,9-diazaspiro[5.5]undecan-3-ylmethyl)-4-ethyl-pyrrolidin-1-yl]quinoline-8-carbonitrile (Example 15)

To a solution of [trans-1-(8-cyano-5-quinolyl)-4-ethyl-pyrrolidin-3-yl]methyl trifluoromethanesulfonate (compound 15e, 29 mg, 70 μmol) and tert-butyl 3,9-diazaspiro[5.5]undecane-3-carboxylate (compound 15f, 18 mg, 70 μmol) in acetonitrile (15 mL) was added K₂CO₃ (38 mg, 281 μmol). After the mixture was heated to reflux for 4 hours, it was diluted with ACN and filtered through celite. The filtrate was concentrated to give a yellow intermediate. The intermediate was dissolved in 5 mL DCM. Then 0.5 mL TFA was added to the solution. After the reaction mixture was stirred for 2 hours at r.t., it was concentrated to afford an oil, which was purified by reverse phase HPLC to give Example 15 (8 mg) as a yellow solid. MS: calc'd 418 (MH⁺), measured 418 (MH⁺). ¹H NMR (400 MHz, METHANOL-d₄) δ=8.88-8.78 (m, 2H), 7.95 (d, J=8.6 Hz, 1H), 7.46 (dd, J=4.2, 8.7 Hz, 1H), 6.80 (d, J=8.6 Hz, 1H), 3.84 (ddd, J=7.2, 9.8, 16.6 Hz, 2H), 3.71-3.60 (m, 1H), 3.48 (t, J=9.2 Hz, 1H), 3.18-3.07 (m, 4H), 2.76-2.45 (m, 5H), 2.31 (br s, 1H), 2.07-1.91 (m, 1H), 1.84-1.56 (m, 10H), 1.50-1.33 (m, 1H), 1.03 (t, J=7.4 Hz, 3H).

### Example 16

### 8-[7-(3,9-Diazaspiro[5.5]undecan-3-ylmethyl)-5-azaspiro[2.4]heptan-5-yl]quinoxaline-5-carbonitrile

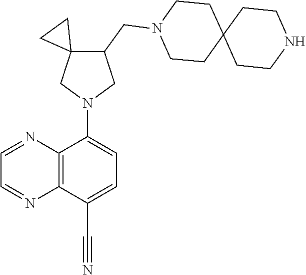 [SMILES: N#Cc1ccc(N2CC(CN3CCC4(CCNCC4)CC3)C3(CC3)C2)c2nccnc12]

The title compound was prepared in analogy to the preparation of Example 3 by using tert-butyl 3,9-diazaspiro[5.5]undecane-3-carboxylate instead of tert-butyl piperazine-1-carboxylate (compound 3g). Example 16 (21 mg) was obtained as a yellow solid. MS: calc'd 417 (MH⁺), measured 417 (MH⁺). ¹H NMR (400 MHz, METHANOL-d₄) δ=8.70 (d, J=1.71 Hz, 1H), 8.61 (d, J=1.71 Hz, 1H), 7.80 (d, J=8.56 Hz, 1H), 6.63 (d, J=8.80 Hz, 1H), 4.21-4.29 (m, 1H), 4.11-4.20 (m, 1H), 4.05 (d, J=11.74 Hz, 1H), 3.48-3.60 (m, 2H), 3.37-3.45 (m, 1H), 3.29 (dd, J=10.76, 13.20 Hz, 1H), 3.06-3.16 (m, 6H), 2.99 (t, J=13.20 Hz, 1H), 2.31-2.41 (m, 1H), 1.77-1.99 (m 4H), 1.52-1.74 (m 4H), 0.64-0.83 (m 4H).

### Example 17

### 8-[(3S,4R)-3-(3,9-diazaspiro[5.5]undecan-3-ylmethyl)-4-methyl-pyrrolidin-1-yl]quinoxaline-5-carbonitrile

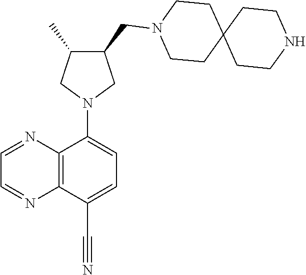 [SMILES: C[C@H]1CN(c2ccc(C#N)c3nccnc23)C[C@@H]1CN1CCC2(CCNCC2)CC1]

The title compound was prepared in analogy to the preparation of Example 1 by using ((3R,4R)-4-methylpyrrolidin-3-yl)methanol hydrochloride salt (Pharmablock, PBXA3260-1) instead of ((3R,4R)-4-(trifluoromethyl)pyrrolidin-3-yl)methanol hydrochloride salt (compound 1b). Example 17 (18 mg) was obtained as a yellow solid. MS: calc'd 405 (MH⁺), measured 405 (MH⁺). ¹H NMR (400 MHz, METHANOL-d₄) δ=8.82 (d, J=1.8 Hz, 1H), 8.73 (d, J=1.7 Hz, 1H), 7.92 (d, J=8.7 Hz, 1H), 6.72 (d, J=8.8 Hz, 1H), 4.39 (dd, J=7.2, 11.7 Hz, 1H), 4.15 (dd, J=7.4, 11.2 Hz, 1H), 3.94-3.81 (m, 1H), 3.73-3.41 (m, 4H), 3.28-3.07 (m, 7H), 2.48-2.33 (m, 1H), 2.25-2.12 (m, 1H), 2.09-1.61 (m, 8H), 1.26 (d, J=6.5 Hz, 3H).

### Example 18

### 8-[7-[[(3aR,6aS)-2,3,3a,4,6,6a-hexahydro-H-pyrrolo[3,4-c]pyrrol-5-yl]methyl]-5-azaspiro[2.4]heptan-5-yl]quinoxaline-5-carbonitrile

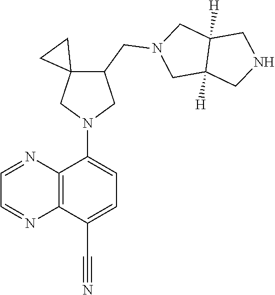 [SMILES: N#Cc1ccc(N2CC(CN3C[C@H]4CNC[C@H]4C3)C3(CC3)C2)c2nccnc12]

The title compound was prepared in analogy to the preparation of Example 3 by using tert-butyl-(3aS,6aR)-2,3,3a,4,6,6a-hexahydro-1H-pyrrolo[3,4-c]pyrrole-5-carboxylate instead of tert-butyl piperazine-1-carboxylate (compound 3g). Example 18 (23 mg) was obtained as a yellow solid. MS: calc'd 375 (MH⁺), measured 375 (MH⁺). ¹H NMR (400 MHz, METHANOL-d₄) δ=8.83 (d, J=1.71 Hz, 1H), 8.75 (d, J=1.71 Hz, 1H), 7.97 (d, J=8.68 Hz, 1H), 6.78 (d, J=8.80 Hz, 1H), 4.18-4.29 (m, 1H), 4.08-4.18 (m, 2H), 3.79 (d, J=11.62 Hz, 1H), 3.52 (td, J=7.24, 11.55 Hz, 3H), 3.03-3.27 (m, 6H), 2.97 (m, 2H), 2.66 (d, J=16.14 Hz, 1H), 2.18-2.36 (m, 1H), 0.80-0.92 (m, 1H), 0.63-0.80 (m, 3H)

### Example 19

### 5-[Trans-3-(2,7-diazaspiro[4.4]nonan-2-ylmethyl)-4-(trifluoromethyl)pyrrolidin-1-yl]-8-(trifluoromethyl)quinoxaline

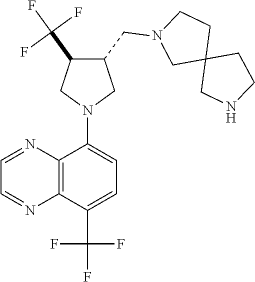 [SMILES: FC(F)(F)c1ccc(N2C[C@@H](CN3CCC4(CCNC4)C3)[C@H](C(F)(F)F)C2)c2nccnc12]

The title compound was prepared in analogy to the preparation of Example 15 by using methyl tans-4-(trifluoromethyl)pyrrolidine-3-carboxylate hydrochloride salt (Pharmablock, PBXA3194-1) and 5-bromo-8-(trifluoromethyl)quinoxaline and tert-butyl 2,7-diazaspiro[4.4]nonane-2-carboxylate instead of ethyl trans-4-ethylpyrrolidine-3-carboxylate hydrochloride salt (compound 15b) and 5-bromoquinoline-8-carbonitrile (compound 15a). tert-butyl 3,9-diazaspiro[5.5]undecane-3-carboxylate (compound 15f) Example 19 (4 mg) was obtained as a yellow solid. MS: calc'd 474 (MH⁺), measured 474 (MH⁺). ¹H NMR (400 MHz, METHANOL-d₄) δ=8.83-8.66 (m, 2H), 7.87 (d, J=8.7 Hz, 1H), 6.79 (d, J=8.6 Hz, 1H), 4.27-4.08 (m, 3H), 3.78 (dd, J=6.7, 10.9 Hz, 1H), 3.46 (br d, J=7.0 Hz, 3H), 3.43-3.25 (m, 6H), 3.16-2.86 (m, 2H), 2.24-2.00 (m, 5H).

### Example 20

### 5-[7-(Piperazin-1-ylmethyl)-5-azaspiro[2.4]heptan-5-yl]-8-(trifluoromethyl)quinoxaline

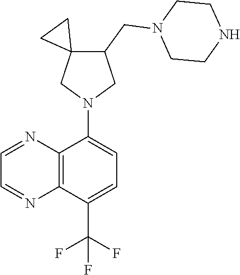 [SMILES: FC(F)(F)c1ccc(N2CC(CN3CCNCC3)C3(CC3)C2)c2nccnc12]

The title compound was prepared in analogy to the preparation of Example 3 by using 5-bromo-8-(trifluoromethyl)quinoxaline instead of 8-bromoquinoxaline-5-carbonitrile (compound 1a). Example 20 (13 mg) was obtained as a yellow solid. MS: calc'd 392 (MH⁺), measured 392 (MH⁺). ¹H NMR (400 MHz, METHANOL-d₄) δ=8.68 (d, J=1.71 Hz, 1H), 8.62 (d, J=1.71 Hz, 1H), 7.80 (d, J=8.68 Hz, 1H), 6.63 (d, J=8.68 Hz, 1H), 3.98-4.08 (m, 2H), 3.92 (dd, J=3.42, 11.25 Hz, 1H), 3.58 (d, J=11.37 Hz, 1H), 3.11-3.18 (m, 4H), 2.83 (d, J=11.49 Hz, 2H), 2.55-2.71 (m, 3H), 2.41-2.51 (m, 1H), 2.09-2.22 (m, 1H), 0.71-0.80 (m, 1H), 0.53-0.66 (m, 3H).

### Example 21

### 3-[[Trans-4-methyl-1-[8-(trifluoromethyl)quinoxalin-5-yl]pyrrolidin-3-yl]methyl]-3,9-diazaspiro[5.5]undecane

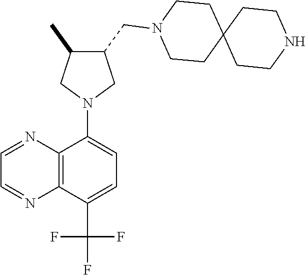 [SMILES: C[C@@H]1CN(c2ccc(C(F)(F)F)c3nccnc23)C[C@H]1CN1CCC2(CCNCC2)CC1]

The title compound was prepared in analogy to the preparation of Example 15 by using methyl trans-4-methylpyrrolidine-3-carboxylate (Bepharm, B162777) and 5-bromo-8-(trifluoromethyl)quinoxaline instead of ethyl trans-4-ethylpyrrolidine-3-carboxylate hydrochloride salt (compound 15b) and 5-bromoquinoline-8-carbonitrile (compound 15a). Example 21 (16 mg) was obtained as a yellow solid. MS: calc'd 448 (MH⁺), measured 448 (MH⁺). ¹H NMR (400 MHz, METHANOL-d₄) δ=8.75-8.69 (m, 1H), 8.65 (d, J=1.7 Hz, 1H), 7.82 (d, J=8.8 Hz, 1H), 6.66 (d, J=8.6 Hz, 1H), 4.23 (dd, J=7.3, 11.3 Hz, 1H), 4.00 (dd, J=7.3, 10.9 Hz, 1H), 3.81 (dd, J=8.7, 11.3 Hz, 1H), 3.61-3.30 (m, 5H), 3.16-2.96 (m, 6H), 2.29 (br t, J=9.4 Hz, 1H), 2.15-2.00 (m, 1H), 2.01-1.50 (m, 8H), 1.16 (d, J=6.5 Hz, 3H).

### Example 22

### 8-[7-(2,9-Diazaspiro[4.5]decan-2-ylmethyl)-5-azaspiro[2.4]heptan-5-yl]quinoxaline-5-carbonitrile

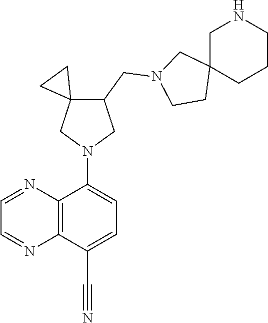 [SMILES: N#Cc1ccc(N2CC(CN3CCC4(CCCNC4)C3)C3(CC3)C2)c2nccnc12]

The title compound was prepared in analogy to the preparation of Example 3 by using tert-butyl 2,7-diazaspiro[4.5]decane-7-carboxylate instead of tert-butyl piperazine-1-carboxylate (compound 3g). Example 22 (20 mg) was obtained as a yellow solid. MS: calc'd 403 (MH⁺), measured 403 (MH⁺). ¹H NMR (400 MHz, METHANOL-d₄) δ=8.84 (d, J=1.71 Hz, 1H), 8.76 (d, J=0.86 Hz, 1H), 7.98 (d, J=8.68 Hz, 1H), 6.78 (d, J=8.80 Hz, 1H), 4.21-4.33 (m, 1H), 4.07-4.20 (m, 2H), 3.71-3.81 (m, 1H), 2.89-3.23 (m, 8H), 2.70-2.85 (m, 2H), 2.30 (m, 1H), 1.72-1.98 (m, 6H), 0.85-0.96 (m, 1H), 0.69-0.81 (m, 3H).

### Example 23

### 5-[(3S,4R)-3-(3,9-diazaspiro[5.5]undecan-3-ylmethyl)-4-methyl-pyrrolidin-1-yl]quinoline-8-carbonitrile

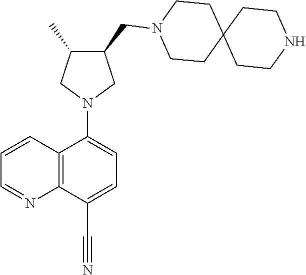 [SMILES: C[C@H]1CN(c2ccc(C#N)c3ncccc23)C[C@@H]1CN1CCC2(CCNCC2)CC1]

The title compound was prepared in analogy to the preparation of Example 1 by using ((3R,4R)-4-methylpyrrolidin-3-yl)methanol hydrochloride salt (Pharmablock, PBXA3260-1) and 5-bromoquinoline-8-carbonitrile instead of ((3R,4R)-4-(trifluoromethyl)pyrrolidin-3-yl)methanol hydrochloride salt (compound 1b) and bromoquinoxaline-5-carbonitrile (compound 1a). Example 23 (18 mg) was obtained as a yellow solid. MS: calc'd 404 (MH⁺), measured 404 (MH⁺). ¹H NMR (400 MHz, METHANOL-d₄) δ=8.83 (dd, J=1.2, 4.3 Hz, 1H), 8.73 (dd, J=1.3, 8.7 Hz, 1H), 7.86 (d, J=8.4 Hz, 1H), 7.43 (dd, J=4.3, 8.7 Hz, 1H), 6.71 (d, J=8.7 Hz, 1H), 4.03 (dd, J=7.0, 10.0 Hz, 1H), 3.82-3.64 (m, 3H), 3.59-3.41 (m, 4H), 3.28-3.06 (m, 6H), 2.55-2.40 (m, 1H), 2.31-2.15 (m, 1H), 2.08-1.64 (m, 8H), 1.25 (d, J=6.5 Hz, 3H).

### Example 24

### 8-[7-(2,8-Diazaspiro[3.5]nonan-2-ylmethyl)-5-azaspiro[2.4]heptan-5-yl]quinoxaline-5-carbonitrile

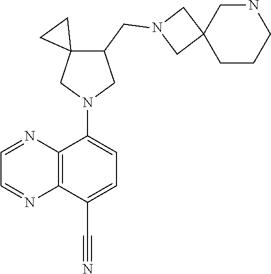 [SMILES: N#Cc1ccc(N2CC(CN3CC4(CCCNC4)C3)C3(CC3)C2)c2nccnc12]

The title compound was prepared in analogy to the preparation of Example 3 by using tert-butyl 2,6-diazaspiro[3.5]nonane-6-carboxylate instead of tert-butyl piperazine-1-carboxylate (compound 3g). Example 24 (18 mg) was obtained as a yellow solid. MS: calc'd 389 (MH⁺), measured 389 (MH⁺). ¹H NMR (400 MHz, METHANOL-d₄) δ=8.82 (d, J=1.59 Hz, 1H), 8.74 (d, J=1.47 Hz, 1H), 7.95 (d, J=8.68 Hz, 1H), 6.74 (d, J=8.80 Hz, 1H), 4.21 (dd, J=6.17, 11.31 Hz, 1H), 4.06 (d, J=11.62 Hz, 2H), 3.68-3.79 (m, 1H), 3.36-3.53 (m, 4H), 3.05-3.19 (m, 3H), 2.68-2.93 (m, 2H), 1.90-2.16 (m, 3H), 1.70-1.89 (m, 3H), 0.81-0.94 (m, 1H), 0.66-0.77 (m, 3H).

### Example 25

### 8-[7-[(3-Amino-8-azabicyclo[3.2.1]octan-8-yl)methyl]-5-azaspiro[2.4]heptan-5-yl]quinoxaline-5-carbonitrile

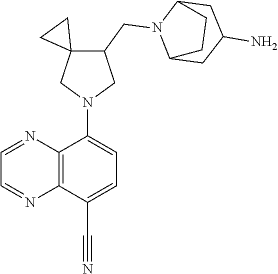 [SMILES: N#Cc1ccc(N2CC(CN3C4CCC3CC(N)C4)C3(CC3)C2)c2nccnc12]

The title compound was prepared in analogy to the preparation of Example 3 by using tert-butyl N-(8-azabicyclo[3.2.1]octan-3-yl)carbamate (PharmaBlock, CAS:132234-69-6) instead of tert-butyl piperazine-1-carboxylate (compound 3g). Example 25 (12 mg) was obtained as a yellow solid. MS: calc'd 389 (MH⁺), measured 389 (MH⁺). ¹H NMR (400 MHz, METHANOL-d₄) δ=8.85 (d, J=1.71 Hz, 1H), 8.75 (d, J=1.71 Hz, 1H), 7.97 (d, J=8.68 Hz, 1H), 6.80 (d, J=8.68 Hz, 1H), 4.13-4.41 (m, 5H), 3.65-3.81 (m, 2H), 3.27-3.31 (m, 2H), 3.03-3.18 (m, 1H), 2.34-2.50 (m, 2H), 2.26 (m, 4H), 2.05-2.17 (m, 2H), 0.94 (d, J=5.62 Hz, 1H), 0.77-0.91 (m, 3H).

### Example 26

### 8-[(3S,4R)-3-[(4-Amino-1-piperidyl)methyl]-4-(trifluoromethyl)pyrrolidin-1-yl]quinoxaline-5-carbonitrile

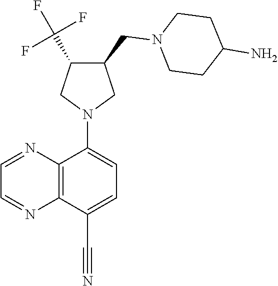 [SMILES: N#Cc1ccc(N2C[C@H](CN3CCC(N)CC3)[C@@H](C(F)(F)F)C2)c2nccnc12]

The title compound was prepared in analogy to the preparation of Example 1 by using tert-butyl N-(4-piperidyl)carbamate (PharmaBlock, CAS:73874-95-0) instead of tert-butyl 3,9-diazaspiro[5.5]undecane-3-carboxylate (compound 1e). Example 26 (11 mg) was obtained as a yellow solid. MS: calc'd 405 (MH⁺), measured 405 (MH⁺). ¹H NMR (400 MHz, METHANOL-d₄) δ=8.88 (d, J=1.8 Hz, 1H), 8.80 (d, J=1.7 Hz, 1H), 8.00 (d, J=8.6 Hz, 1H), 6.84 (d, J=8.7 Hz, 1H), 4.45-4.21 (m, 3H), 3.89 (dd, J=6.4, 11.4 Hz, 1H), 3.81-3.58 (m, 2H), 3.50-3.36 (m, 2H), 3.20-2.86 (m 5H), 2.24 (br d, J=11.7 Hz, 2H), 2.08-1.89 (m 2H).

### Example 27

### 5-[Trans-3-(3,9-diazaspiro[5.5]undecan-3-ylmethyl)-4-(difluoromethyl)pyrrolidin-1-yl]quinoline-8-carbonitrile

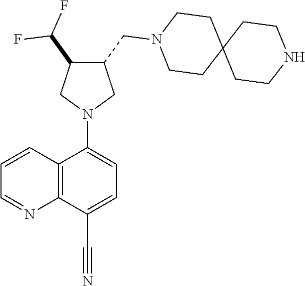 [SMILES: N#Cc1ccc(N2C[C@@H](CN3CCC4(CCNCC4)CC3)[C@H](C(F)F)C2)c2cccnc12]

The title compound was prepared in analogy to the preparation of Example 15 by using ethyl trans-4-difluoromethyl-pyrrolidine-3-carboxylate hydrochloride (Pharmablock, PBXA3200-1) instead of ethyl trans-4-ethylpyrrolidine-3-carboxylate hydrochloride salt (compound 15b). Example 27 (8 mg) was obtained as a yellow solid. MS: calc'd 440 (MH⁺), measured 440 (MH⁺). ¹H NMR (400 MHz, METHANOL-d₄) δ=8.92 (dd, J=1.5, 4.3 Hz, 1H), 8.72 (dd, J=1.5, 8.7 Hz, 1H), 8.02 (d, J=8.3 Hz, 1H), 7.55 (dd, J=4.3, 8.7 Hz, 1H), 6.99 (d, J=8.4 Hz, 1H), 6.38-6.00 (m, 1H), 3.99 (dd, J=7.2, 9.9 Hz, 1H), 3.82-3.63 (m, 8H), 3.63-3.39 (m, 4H), 3.13-2.73 (m, 3H), 2.10-1.57 (m, 8H).

### Example 28

### 5-[Trans-3-cyclopropyl-4-(3,9-diazaspiro[5.5]undecan-3-ylmethyl)pyrrolidin-1-yl]quinoline-8-carbonitrile

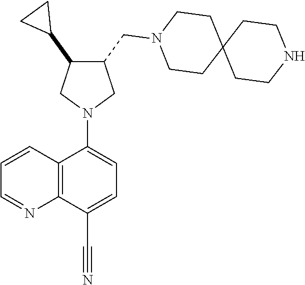 [SMILES: N#Cc1ccc(N2C[C@@H](CN3CCC4(CCNCC4)CC3)[C@H](C3CC3)C2)c2cccnc12]

The title compound was prepared in analogy to the preparation of Example 15 by using ethyl trans-4-cyclopropylpyrrolidine-3-carboxylate hydrochloride (Pharmablock, PBXA3214-1) instead of ethyl trans-4-ethylpyrrolidine-3-carboxylate hydrochloride salt (compound 15b). Example 28 (17 mg) was obtained as a yellow solid. MS: calc'd 430 (MH⁺), measured 430 (MH⁺). ¹H NMR (400 MHz, METHANOL-d₄) δ=8.89-8.78 (m, 2H), 7.94 (d, J=8.6 Hz, 1H), 7.59-7.46 (m, 1H), 6.80 (d, J=8.7 Hz, 1H), 4.06 (dd, J=7.0, 10.0 Hz, 1H), 3.86-3.61 (m, 5H), 3.57-3.42 (m, 1H), 3.28-3.09 (m, 8H), 2.86-2.66 (m, 1H), 2.12-1.43 (m, 9H), 0.90-0.17 (m, 4H).

### Example 29

### 5-[Trans-3-(2,7-diazaspiro[4.4]nonan-2-ylmethyl)-4-ethyl-pyrrolidin-1-yl]quinoline-8-carbonitrile

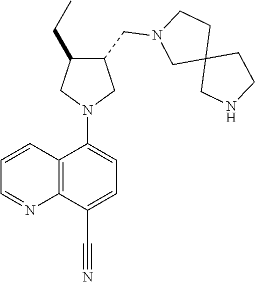 [SMILES: CC[C@@H]1CN(c2ccc(C#N)c3ncccc23)C[C@H]1CN1CCC2(CCNC2)C1]

The title compound was prepared in analogy to the preparation of Example 15 by using tert-butyl 2,7-diazaspiro[4.4]nonane-2-carboxylate instead of tert-butyl 3,9-diazaspiro[5.5]undecane-3-carboxylate (compound 15f). Example 29 (4 mg) was obtained as a yellow solid. MS: calc'd 390 (MH⁺), measured 390 (MH⁺). ¹H NMR (400 MHz, METHANOL-d₄) δ=8.89-8.71 (m, 2H), 7.91 (br d, J=7.9 Hz, 1H), 7.45 (dd, J=4.0, 8.5 Hz, 1H), 6.76 (br d, J=7.9 Hz, 1H), 3.98-3.73 (m, 2H), 3.73-3.57 (m, 1H), 3.55-3.40 (m, 1H), 3.27-3.10 (m, 4H), 3.07-2.66 (m, 6H), 2.37-1.94 (m, 6H), 1.87-1.69 (m, 1H), 1.52-1.35 (m, 1H), 1.04 (t, J=7.5 Hz, 3H).

### Example 30

### 8-[(3S,4R)-3-(2,7-diazaspiro[4.4]nonan-2-ylmethyl)-4-methyl-pyrrolidin-1-yl]quinoxaline-5-carbonitrile

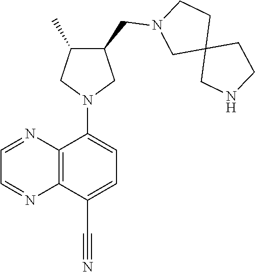 [SMILES: C[C@H]1CN(c2ccc(C#N)c3nccnc23)C[C@@H]1CN1CCC2(CCNC2)C1]

The title compound was prepared in analogy to the preparation of Example 1 by using ((3R,4R)-4-methylpyrrolidin-3-yl)methanol hydrochloride salt (Pharmablock, PBXA3260-1) and tert-butyl 2,7-diazaspiro[4.4]nonane-2-carboxylate instead of ((3R,4R)-4-(trifluoromethyl)pyrrolidin-3-yl)methanol hydrochloride salt (compound 1b) and tert-butyl 3,9-diazaspiro[5.5]undecane-3-carboxylate (compound 1e). Example 30 (19 mg) was obtained as a yellow solid. MS: calc'd 377 (MH⁺), measured 377 (MH⁺). ¹H NMR (400 MHz, METHANOL-d₄) δ=8.82 (d, J=1.8 Hz, 1H), 8.73 (d, J=1.7 Hz, 1H), 7.92 (d, J=8.3 Hz, 1H), 6.72 (d, J=8.4 Hz, 1H), 4.36 (br dd, J=7.5, 11.5 Hz, 1H), 4.16 (br dd, J=7.4, 11.2 Hz, 1H), 3.99-3.82 (m, 2H), 3.66-3.35 (m, 10H), 2.42-2.08 (m, 6H), 1.26 (d, J=6.5 Hz, 3H).

### Example 31

### 3-[[5-[8-(Trifluoromethyl)quinoxalin-5-yl]-5-azaspiro[2.4]heptan-7-yl]methyl]-3,9-diazaspiro[5.5]undecane

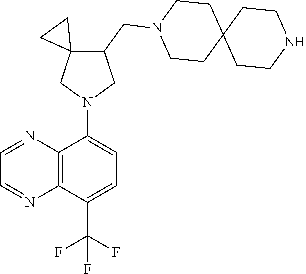 [SMILES: FC(F)(F)c1ccc(N2CC(CN3CCC4(CCNCC4)CC3)C3(CC3)C2)c2nccnc12]

The title compound was prepared in analogy to the preparation of Example 3 by using 5-bromo-8-(trifluoromethyl)quinoxaline and tert-butyl 3,9-diazaspiro[5.5]undecane-3-carboxylate instead of 8-bromoquinoxaline-5-carbonitrile (compound 1a) and tert-butyl piperazine-1-carboxylate (compound 3g). Example 31 (19 mg) was obtained as a yellow solid. MS: calc'd 460 (MH⁺), measured 460 (MH⁺). ¹H NMR (400 MHz, METHANOL-d₄) δ=8.71 (d, J=1.59 Hz, 1H), 8.65 (d, J=1.59 Hz, 1H), 7.83 (d, J=8.68 Hz, 1H), 6.68 (d, J=8.56 Hz, 1H), 4.22 (dd, J=5.75, 11.37 Hz, 1H), 4.06 (br d, J=11.49 Hz, 2H), 3.49 (d, J=11.13 Hz, 2H), 3.34-3.42 (m, 1H), 3.24-3.30 (m, 1H), 2.90-3.15 (m, 7H), 2.26-2.37 (m, 1H), 1.74-1.97 (m, 4H), 1.53-1.73 (m, 4H), 0.65-0.81 (m, 4H).

### Example 32

### 8-[7-(2,8-Diazaspiro[4.5]decan-2-ylmethyl)-5-azaspiro[2.4]heptan-5-yl]quinoxaline-5-carbonitrile

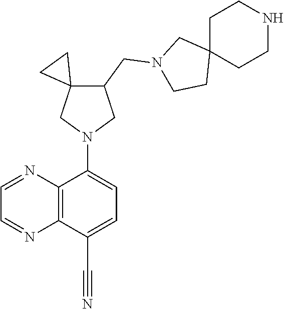 [SMILES: N#Cc1ccc(N2CC(CN3CCC4(CCNCC4)C3)C3(CC3)C2)c2nccnc12]

The title compound was prepared in analogy to the preparation of Example 3 by using tert-butyl 2,8-diazaspiro[4.5]decane-8-carboxylate instead of tert-butyl piperazine-1-carboxylate (compound 3g). Example 32 (29 mg) was obtained as a yellow solid. MS: calc'd 403 (MH⁺), measured 403 (MH⁺). ¹H NMR (400 MHz, METHANOL-d₄) δ=8.81 (d, J=1.59 Hz, 1H), 8.71 (d, J=1.59 Hz, 1H), 7.89 (d, J=8.68 Hz, 1H), 6.72 (d, J=8.68 Hz, 1H), 4.23-4.41 (m, 2H), 4.13 (d, J=11.74 Hz, 1H), 3.84 (d, J=10.27 Hz, 2H), 3.69 (d, J=11.62 Hz, 1H), 3.49-3.58 (m, 1H), 3.04-3.28 (m, 6H), 2.40-2.51 (m, 1H), 1.85-2.29 (m, 7H), 0.89-1.00 (m, 1H), 0.75-0.89 (m, 3H).

### Example 33

### 5-[Trans-3-methyl-4-[[4-(1-piperidyl)-1-piperidyl]methyl]pyrrolidin-1-yl]-8-(trifluoromethyl)quinoxaline

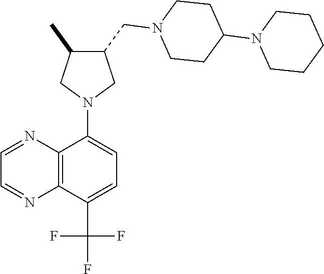 [SMILES: C[C@@H]1CN(c2ccc(C(F)(F)F)c3nccnc23)C[C@H]1CN1CCC(N2CCCCC2)CC1]

The title compound was prepared in analogy to the preparation of Example 15 by using methyl trans-4-methylpyrrolidine-3-carboxylate (Bepharm, B162777) and 5-bromo-8-(trifluoromethyl)quinoxaline and 1-(4-piperidyl)piperidine instead of ethyl trans-4-ethylpyrrolidine-3-carboxylate hydrochloride salt (compound 15b) and 5-bromoquinoline-8-carbonitrile (compound 15a) and tert-butyl 3,9-diazaspiro[5.5]undecane-3-carboxylate (compound 15f). Example 33 (11 mg) was obtained as a yellow solid. MS: calc'd 462 (MH⁺), measured 462 (MH⁺). ¹H NMR (400 MHz, METHANOL-d₄) δ=8.70 (d, J=1.7 Hz, 1H), 8.64 (d, J=1.7 Hz, 1H), 7.80 (d, J=8.7 Hz, 1H), 6.65 (d, J=8.8 Hz, 1H), 4.21 (dd, J=7.3, 11.4 Hz, 1H), 4.01 (dd, J=7.3, 11.0 Hz, 1H), 3.91-3.67 (m, 3H), 3.56-3.26 (m, 4H), 3.16-2.86 (m, 6H), 2.40-2.25 (m, 3H), 2.17-1.63 (m, 8H), 1.20-1.09 (m, 3H).

### Example 34

### 5-[Trans-3-(2,7-diazaspiro[4.4]nonan-2-ylmethyl)-4-(difluoromethyl)pyrrolidin-1-yl]quinoline-8-carbonitrile

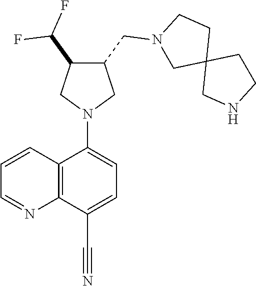 [SMILES: N#Cc1ccc(N2C[C@@H](CN3CCC4(CCNC4)C3)[C@H](C(F)F)C2)c2cccnc12]

The title compound was prepared in analogy to the preparation of Example 15 by using ethyl trans-4-difluoromethyl-pyrrolidine-3-carboxylate hydrochloride (Pharmablock, PBXA3200-1) and tert-butyl 2,7-diazaspiro[4.4]nonane-2-carboxylate instead of ethyl trans-4-ethylpyrrolidine-3-carboxylate hydrochloride salt (compound 15b) and tert-butyl 3,9-diazaspiro[5.5]undecane-3-carboxylate (compound 15f). Example 34 (10 mg) was obtained as a yellow solid. MS: calc'd 412 (MH⁺), measured 412 (MH⁺). ¹H NMR (400 MHz, METHANOL-d₄) δ=8.91 (dd, J=1.5, 4.3 Hz, 1H), 8.72 (dd, J=1.0, 8.7 Hz, 1H), 8.01 (d, J=8.2 Hz, 1H), 7.54 (dd, J=4.3, 8.7 Hz, 1H), 6.99 (d, J=8.3 Hz, 1H), 6.35-6.00 (m, 1H), 4.06-3.92 (m, 1H), 3.81-3.66 (m, 3H), 3.64-3.53 (m, 4H), 3.50-3.36 (m, 5H), 3.04-2.67 (m, 3H), 2.38-2.08 (m, 4H).

### Example 35

### 5-[(3S,4R)-3-(2,7-diazaspiro[4.4]nonan-2-ylmethyl)-4-methyl-pyrrolidin-1-yl]quinoline-8-carbonitrile

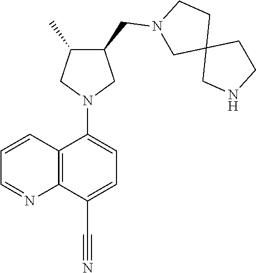 [SMILES: C[C@H]1CN(c2ccc(C#N)c3ncccc23)C[C@@H]1CN1CCC2(CCNC2)C1]

The title compound was prepared in analogy to the preparation of Example 1 by using ((3R,4R)-4-methylpyrrolidin-3-yl)methanol hydrochloride salt (Pharmablock, PBXA3260-1) and 5-bromoquinoline-8-carbonitrile and tert-butyl 2,7-diazaspiro[4.4]nonane-2-carboxylate instead of ((3R,4R)-4-(trifluoromethyl)pyrrolidin-3-yl)methanol hydrochloride salt (compound 1b) and bromoquinoxaline-5-carbonitrile (compound 1a) and tert-butyl 3,9-diazaspiro[5.5]undecane-3-carboxylate (compound 1e). Example 35 (35 mg) was obtained as a yellow solid. MS: calc'd 376 (MH⁺), measured 376 (MH⁺). ¹H NMR (400 MHz, METHANOL-d₄) δ=8.84 (dd, J=1.2, 4.3 Hz, 1H), 8.74 (d, J=8.7 Hz, 1H), 7.87 (d, J=8.6 Hz, 1H), 7.43 (dd, J=4.3, 8.8 Hz, 1H), 6.71 (dd, J=1.3, 8.6 Hz, 1H), 4.08-3.95 (m, 1H), 3.85-3.71 (m, 3H), 3.61-3.35 (m, 10H), 2.47-2.08 (m, 6H), 1.25 (d, J=6.5 Hz, 3H).

### Example 36

### 5-[Trans-3-(3,9-diazaspiro[5.5]undecan-3-ylmethyl)-4-isopropyl-pyrrolidin-1-yl]quinoline-8-carbonitrile

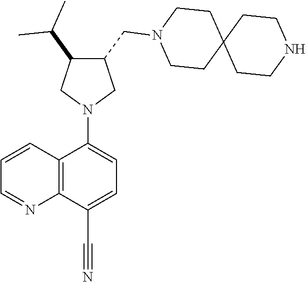 [SMILES: CC(C)[C@@H]1CN(c2ccc(C#N)c3ncccc23)C[C@H]1CN1CCC2(CCNCC2)CC1]

The title compound was prepared in analogy to the preparation of Example 15 by using methyl trans-4-isopropyl-pyrrolidine-3-carboxylate hydrochloride salt (CAS: 1820575-33-4) instead of ethyl trans-4-ethylpyrrolidine-3-carboxylate hydrochloride salt (compound 15b). Example 36 (31 mg) was obtained as a yellow solid. MS: calc'd 432 (MH⁺), measured 432 (MH⁺). ¹H NMR (400 MHz, METHANOL-d₄) δ=8.87 (dd, J=1.5, 4.2 Hz, 1H), 8.79 (d, J=7.3 Hz, 1H), 7.98 (d, J=8.4 Hz, 1H), 7.49 (dd, J=4.2, 8.7 Hz, 1H), 6.87 (d, J=8.4 Hz, 1H), 3.87-3.73 (m, 1H), 3.72-3.43 (m, 4H), 3.23-3.09 (m, 4H), 2.95-2.44 (m, 7H), 2.07-1.62 (m, 9H), 1.12-0.95 (m, 6H).

### Example 37

### 8-[3-(3,9-Diazaspiro[5.5]undecan-3-ylmethyl)pyrrolidin-1-yl]quinoxaline-5-carbonitrile

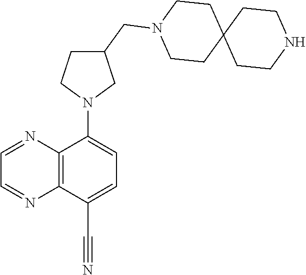 [SMILES: N#Cc1ccc(N2CCC(CN3CCC4(CCNCC4)CC3)C2)c2nccnc12]

The title compound was prepared in analogy to the preparation of Example 1 by using pyrrolidin-3-ylmethanol instead of ((3R,4R)-4-(trifluoromethyl)pyrrolidin-3-yl)methanol hydrochloride salt (compound 1b). Example 37 (23 mg) was obtained as a yellow solid. MS: calc'd 391 (MH⁺), measured 391 (MH⁺). ¹H NMR (400 MHz, METHANOL-d₄) δ=8.82 (d, J=1.71 Hz, 1H), 8.73 (d, J=1.83 Hz, 1H), 7.92 (d, J=8.68 Hz, 1H), 6.73 (d, J=8.68 Hz, 1H), 4.31 (dd, J=7.21, 11.62 Hz, 1H), 3.90-4.10 (m, 2H), 3.84 (dd, J=8.25, 11.55 Hz, 1H), 3.63 (t, J=13.27 Hz, 2H), 3.42 (d, J=6.85 Hz, 2H), 3.14-3.29 (m, 6H), 2.85-2.97 (m, 1H), 2.29-2.46 (m, 1H), 2.01-2.16 (m, 2H), 1.89-2.01 (m, 3H), 1.79-1.89 (m, 2H), 1.74 (m, 2H).

### Example 38

### 5-[7-(3,9-Diazaspiro[5.5]undecan-3-ylmethyl)-5-azaspiro[2.4]heptan-5-yl]quinoline-8-carbonitrile

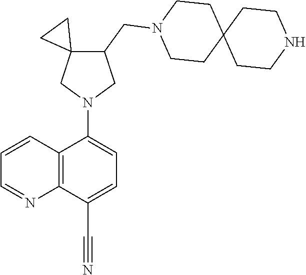 [SMILES: N#Cc1ccc(N2CC(CN3CCC4(CCNCC4)CC3)C3(CC3)C2)c2cccnc12]

The title compound was prepared in analogy to the preparation of Example 3 by using tert-butyl 3,9-diazaspiro[5.5]undecane-3-carboxylate and 5-bromoquinoline-8-carbonitrile instead of tert-butyl piperazine-1-carboxylate (compound 3g) and bromoquinoxaline-5-carbonitrile (compound 1a). Example 38 (32 mg) was obtained as a yellow solid. MS: calc'd 416 (MH⁺), measured 416 (MH⁺). ¹H NMR (400 MHz, METHANOL-d₄) δ=8.75-8.78 (m, 1H), 8.71 (dd, J=1.34, 8.80 Hz, 1H), 7.83 (d, J=8.44 Hz, 1H), 7.38 (dd, J=4.34, 8.74 Hz, 1H), 6.72 (d, J=8.68 Hz, 1H), 4.12 (dd, J=5.87, 10.27 Hz, 1H), 3.91 (d, J=10.03 Hz, 1H), 3.79 (dd, J=3.06, 10.27 Hz, 1H), 3.51 (br d, J=12.47 Hz, 1H), 3.39 (br d, J=11.37 Hz, 1H), 3.25-3.35 (m, 2H), 3.05-3.16 (m, 6H), 2.99 (t, J=12.35 Hz, 1H), 2.35-2.46 (m, 1H), 1.77-1.97 (m, 4H), 1.49-1.72 (m, 4H), 0.63-0.86 (m, 4H).

### Example 39

### 8-[7-[(Azepan-4-ylamino)methyl]-5-azaspiro[2.4]heptan-5-yl]quinoxaline-5-carbonitrile

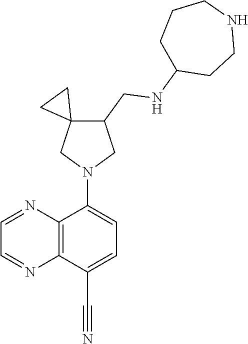 [SMILES: N#Cc1ccc(N2CC(CNC3CCCNCC3)C3(CC3)C2)c2nccnc12]

The title compound was prepared in analogy to the preparation of Example 3 by using tert-butyl 4-aminoazepane-1-carboxylate instead of tert-butyl piperazine-1-carboxylate (compound 3g). Example 39 (25 mg) was obtained as a yellow solid. MS: calc'd 377 (MH⁺), measured 377 (MH⁺). ¹H NMR (400 MHz, METHANOL-d₄) δ=8.79-8.86 (m 1H), 8.74 (s, 1H), 7.94 (dd, J=1.41, 8.62 Hz, 1H), 6.76 (d, J=8.68 Hz, 1H), 4.31-4.42 (m, 1H), 4.17-4.31 (m, 2H), 3.65 (d, J=11.74 Hz, 1H), 3.42-3.57 (m, 2H), 3.12-3.27 (m, 5H), 2.25-2.50 (m, 3H), 2.04-2.18 (m, 2H), 1.69-1.97 (m, 2H), 0.90-1.01 (m, 1H), 0.69-0.90 (m, 3H).

### Example 40

### N-[Trans-4-methyl-1-[8-(trifluoromethyl)quinoxalin-5-yl]pyrrolidin-3-yl]methyl]azepan-4-amine

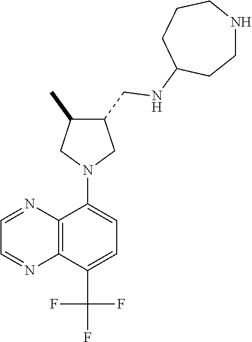 [SMILES: C[C@@H]1CN(c2ccc(C(F)(F)F)c3nccnc23)C[C@H]1CNC1CCCNCC1]

The title compound was prepared in analogy to the preparation of Example 15 by using methyl trans-4-methylpyrrolidine-3-carboxylate (Bepharm, B162777) and 5-bromo-8-(trifluoromethyl)quinoxaline and tert-butyl 4-aminoazepane-1-carboxylate instead of ethyl trans-4-ethylpyrrolidine-3-carboxylate hydrochloride salt (compound 15b) and 5-bromoquinoline-8-carbonitrile (compound 15a) and tert-butyl 3,9-diazaspiro[5.5]undecane-3-carboxylate (compound 15f). Example 40 (14 mg) was obtained as a yellow solid. MS: calc'd 407 (MH⁺), measured 407 (MH⁺). ¹H NMR (400 MHz, METHANOL-d₄) δ=8.86-8.73 (m, 2H), 7.94 (d, J=8.7 Hz, 1H), 6.78 (d, J=8.7 Hz, 1H), 4.32 (br dd, J=6.7, 11.6 Hz, 1H), 4.12 (br dd, J=6.9, 11.1 Hz, 1H), 3.98-3.83 (m, 2H), 3.66-3.48 (m, 3H), 3.25-3.07 (m, 2H), 2.57-1.75 (m, 10H), 1.27 (d, J=6.1 Hz, 3H).

### Example 41

### 5-[7-[[(3aR,6aS)-2,3,3a,4,6,6a-hexahydro-H-pyrrolo[3,4-c]pyrrol-5-yl]methyl]-5-azaspiro[2.4]heptan-5-yl]-8-(trifluoromethyl)quinoxaline

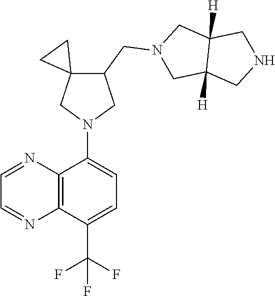 [SMILES: FC(F)(F)c1ccc(N2CC(CN3C[C@H]4CNC[C@H]4C3)C3(CC3)C2)c2nccnc12]

The title compound was prepared in analogy to the preparation of Example 3 by using tert-butyl (3aR,6aS)-2,3,3a,4,6,6a-hexahydro-1H-pyrrolo[3,4-c]pyrrole-5-carboxylate and 5-bromo-8-(trifluoromethyl)quinoxaline instead of tert-butyl piperazine-1-carboxylate (compound 3g) and 8-bromoquinoxaline-5-carbonitrile (compound 1a). Example 41 (21 mg) was obtained as a yellow solid. MS: calc'd 418 (MH⁺), measured 418 (MH⁺). ¹H NMR (400 MHz, METHANOL-d₄) δ=8.71 (d, J=1.71 Hz, 1H), 8.64 (d, J=1.71 Hz, 1H), 7.82 (d, J=8.68 Hz, 1H), 6.67 (d, J=8.56 Hz, 1H), 4.19 (dd, J=5.69, 11.31 Hz, 1H), 3.96-4.11 (m, 2H), 3.52 (d, J=11.25 Hz, 2H), 3.35-3.47 (m, 3H), 3.20-3.27 (m, 6H), 2.96-3.09 (m, 2H), 2.20-2.31 (m, 1H), 0.73-0.81 (m, 1H), 0.61-0.73 (m, 3H).

### Example 42

### 8-[7-[(6-Amino-2-azaspiro[3.3]heptan-2-yl)methyl]-5-azaspiro[2.4]heptan-5-yl]quinoxaline-5-carbonitrile

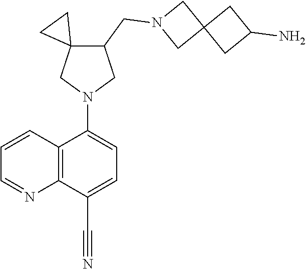 [SMILES: N#Cc1ccc(N2CC(CN3CC4(CC(N)C4)C3)C3(CC3)C2)c2cccnc12]

The title compound was prepared in analogy to the preparation of Example 3 by using tert-butyl N-(2-azaspiro[3.3]heptan-6-yl)carbamate instead of tert-butyl piperazine-1-carboxylate (compound 3g). Example 42 (20 mg) was obtained as a yellow solid. MS: calc'd 375 (MH⁺), measured 375 (MH⁺). ¹H NMR (400 MHz, METHANOL-d₄) δ=8.83 (d, J=1.47 Hz, 1H), 8.75 (d, J=1.47 Hz, 1H), 7.96 (d, J=8.68 Hz, 1H), 6.75 (d, J=8.68 Hz, 1H), 4.25 (dd, J=6.24, 11.74 Hz, 1H), 4.03-4.16 (m, 2H), 3.93 (s, 2H), 3.85 (s, 2H), 3.64-3.76 (m, 2H), 3.00-3.11 (m, 1H), 2.87-2.99 (m, 1H), 2.61-2.75 (m, 2H), 2.31-2.43 (m, 2H), 2.13 (d, J=3.79 Hz, 1H), 0.82-0.96 (m, 1H), 0.70-0.80 (m, 3H).

### Example 43

### 5-[7-(2,7-Diazaspiro[4.4]nonan-2-ylmethyl)-5-azaspiro[2.4]heptan-5-yl]quinoline-8-carbonitrile

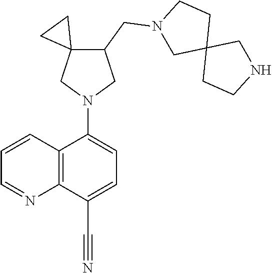 [SMILES: N#Cc1ccc(N2CC(CN3CCC4(CCNC4)C3)C3(CC3)C2)c2cccnc12]

The title compound was prepared in analogy to the preparation of Example 3 by using tert-butyl 2,7-diazaspiro[4.4]nonane-2-carboxylate and 5-bromoquinoline-8-carbonitrile instead of tert-butyl piperazine-1-carboxylate (compound 3g) and bromoquinoxaline-5-carbonitrile (compound 1a). Example 43 (29 mg) was obtained as a yellow solid. MS: calc'd 388 (MH⁺), measured 388 (MH⁺). ¹H NMR (400 MHz, METHANOL-d₄) δ=8.86-8.90 (m, 1H), 8.82 (dd, J=1.41, 8.74 Hz, 1H), 7.93 (d, J=8.44 Hz, 1H), 7.49 (dd, J=4.34, 8.74 Hz, 1H), 6.82 (d, J=8.56 Hz, 1H), 4.23 (dd, J=5.87, 10.27 Hz, 1H), 3.90-4.04 (m, 3H), 3.56 (t, J=11.98 Hz, 2H), 3.37-3.50 (m, 6H), 3.22-3.31 (m, 2H), 2.49 (m, 1H), 2.11-2.34 (m, 4H), 0.76-0.99 (m, 4H).

### Example 44

### 5-[Trans-3-(2,7-diazaspiro[4.4]nonan-2-ylmethyl)-4-isopropyl-pyrrolidin-1-yl]quinoline-8-carbonitrile

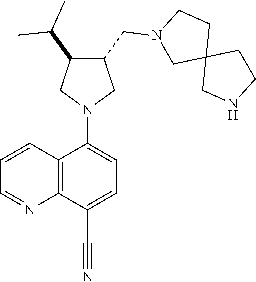 [SMILES: CC(C)[C@@H]1CN(c2ccc(C#N)c3ncccc23)C[C@H]1CN1CCC2(CCNC2)C1]

The title compound was prepared in analogy to the preparation of Example 15 by using methyl trans-4-isopropyl-pyrrolidine-3-carboxylate hydrochloride salt (CAS: 1820575-33-4) and tert-butyl 2,7-diazaspiro[4.4]nonane-2-carboxylate instead of ethyl trans-4-ethylpyrrolidine-3-carboxylate hydrochloride salt (compound 15b) and tert-butyl 3,9-diazaspiro[5.5]undecane-3-carboxylate (compound 15f). Example 44 (20 mg) was obtained as a yellow solid. MS: calc'd 404 (MH⁺), measured 404 (MH⁺). ¹H NMR (400 MHz, METHANOL-d₄) δ=8.90-8.74 (m, 2H), 7.97 (d, J=8.4 Hz, 1H), 7.48 (dd, J=4.2, 8.7 Hz, 1H), 6.86 (d, J=8.6 Hz, 1H), 3.82-3.60 (m, 3H), 3.55-3.43 (m, 1H), 3.22-3.07 (m, 3H), 2.89-2.33 (m, 8H), 2.12-1.80 (m, 6H), 1.11-0.95 (m, 6H).

### Example 45

### 8-[(3S,4R)-3-(2,9-diazaspiro[5.5]undecan-9-ylmethyl)-4-(trifluoromethyl)pyrrolidin-1-yl]quinoxaline-5-carbonitrile

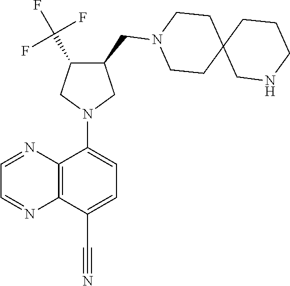 [SMILES: N#Cc1ccc(N2C[C@H](CN3CCC4(CCCNC4)CC3)[C@@H](C(F)(F)F)C2)c2nccnc12]

The title compound was prepared in analogy to the preparation of Example 1 by using tert-butyl 2,9-diazaspiro[5.5]undecane-2-carboxylate instead of tert-butyl 3,9-diazaspiro[5.5]undecane-3-carboxylate (compound 1e). Example 45 (3 mg) was obtained as a yellow solid. MS: calc'd 459 (MH⁺), measured 459 (MH⁺). ¹H NMR (400 MHz, METHANOL-d₄) δ=8.88 (d, J=1.7 Hz, 1H), 8.80 (d, J=1.7 Hz, 1H), 7.99 (d, J=8.6 Hz, 1H), 6.84 (d, J=8.7 Hz, 1H), 4.39 (dd, J=8.3, 12.1 Hz, 2H), 4.33-4.19 (m, 1H), 3.93 (dd, J=6.8, 11.5 Hz, 1H), 3.60-3.43 (m, 4H), 3.23-3.05 (m, 8H), 2.08-1.69 (m, 8H).

### Example 46

### 8-[(3S,4R)-3-(2,8-diazaspiro[4.5]decan-2-ylmethyl)-4-(trifluoromethyl)pyrrolidin-1-yl]quinoxaline-5-carbonitrile

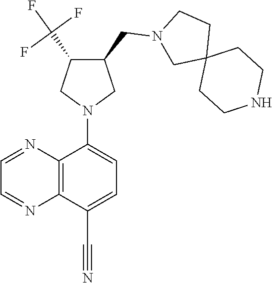 [SMILES: N#Cc1ccc(N2C[C@H](CN3CCC4(CCNCC4)C3)[C@@H](C(F)(F)F)C2)c2nccnc12]

The title compound was prepared in analogy to the preparation of Example 1 by using tert-butyl 2,8-diazaspiro[4.5]decane-8-carboxylate instead of tert-butyl 3,9-diazaspiro[5.5]undecane-3-carboxylate (compound 1e). Example 46 (15 mg) was obtained as a yellow solid. MS: calc'd 445 (MH⁺), measured 445 (MH⁺). ¹H NMR (400 MHz, METHANOL-d₄) δ=8.87 (d, J=1.8 Hz, 1H), 8.80 (d, J=1.7 Hz, 1H), 7.99 (d, J=8.6 Hz, 1H), 6.83 (d, J=8.7 Hz, 1H), 4.45-4.21 (m, 3H), 3.93 (dd, J=6.7, 11.5 Hz, 1H), 3.58 (br d, J=7.1 Hz, 2H), 3.24 (br s, 7H), 3.15-3.01 (m, 1H), 2.28-1.80 (m, 8H).

### Example 47

### 8-[(3S,4R)-3-[(6-amino-2-azaspiro[3.3]heptan-2-yl)methyl]-4-(trifluoromethyl)pyrrolidin-1-yl]quinoxaline-5-carbonitrile

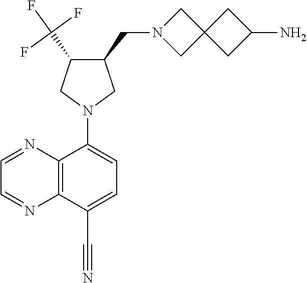 [SMILES: N#Cc1ccc(N2C[C@H](CN3CC4(CC(N)C4)C3)[C@@H](C(F)(F)F)C2)c2nccnc12]

The title compound was prepared in analogy to the preparation of Example 1 by using tert-butyl N-(2-azaspiro[3.3]heptan-6-yl)carbamate instead of tert-butyl 3,9-diazaspiro[5.5]undecane-3-carboxylate (compound 1e). Example 47 (17 mg) was obtained as a yellow solid. MS: calc'd 417 (MH⁺), measured 417 (MH⁺). ¹H NMR (400 MHz, METHANOL-d₄) δ=8.86 (d, J=1.7 Hz, 1H), 8.79 (d, J=1.7 Hz, 1H), 7.98 (d, J=8.6 Hz, 1H), 6.81 (d, J=8.6 Hz, 1H), 4.48-4.14 (m, 7H), 3.88 (dd, J=6.7, 11.4 Hz, 1H), 3.77 (quin, J=8.0 Hz, 1H), 3.64-3.43 (m 2H), 3.26-3.14 (m 1H), 2.99-2.37 (m 5H).

### Example 48

### 8-[(3S,4R)-3-(2,7-diazaspiro[3.5]nonan-2-ylmethyl)-4-(trifluoromethyl)pyrrolidin-1-yl]quinoxaline-5-carbonitrile

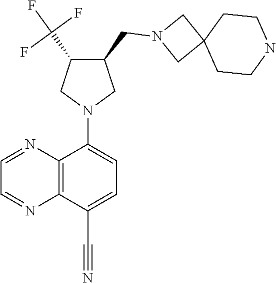 [SMILES: N#Cc1ccc(N2C[C@H](CN3CC4(CCNCC4)C3)[C@@H](C(F)(F)F)C2)c2nccnc12]

The title compound was prepared in analogy to the preparation of Example 1 by using tert-butyl 2,7-diazaspiro[3.5]nonane-7-carboxylate instead of tert-butyl 3,9-diazaspiro[5.5]undecane-3-carboxylate (compound 1e). Example 48 (20 mg) was obtained as a yellow solid. MS: calc'd 431 (MH⁺), measured 431 (MH⁺). ¹H NMR (400 MHz, METHANOL-d₄) δ=8.86 (d, J=1.8 Hz, 1H), 8.78 (d, J=1.8 Hz, 1H), 7.96 (d, J=8.6 Hz, 1H), 6.81 (d, J=8.7 Hz, 1H), 4.40-4.10 (m, 6H), 3.89 (dd, J=6.7, 11.4 Hz, 1H), 3.72-3.53 (m, 2H), 3.31 (td, J=1.6, 3.3 Hz, 6H), 3.00-2.84 (m, 1H), 2.25-2.08 (m, 4H).

### Example 49

### 8-[7-(1-Oxa-4,9-diazaspiro[5.5]undecan-9-ylmethyl)-5-azaspiro[2.4]heptan-5-yl]quinoxaline-5-carbonitrile

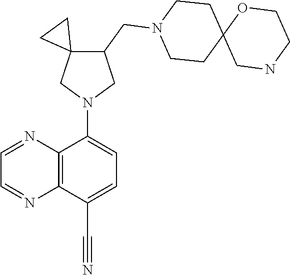 [SMILES: N#Cc1ccc(N2CC(CN3CCC4(CC3)CNCCO4)C3(CC3)C2)c2nccnc12]

The title compound was prepared in analogy to the preparation of Example 3 by using 1-oxa-4,9-diazaspiro[5.5]undecane instead of tert-butyl piperazine-1-carboxylate (compound 3g). Example 49 (27 mg) was obtained as a yellow solid. MS: calc'd 419 (MH⁺), measured 419 (MH⁺). ¹H NMR (400 MHz, METHANOL-d₄) δ=8.71 (d, J=1.71 Hz, 1H), 8.62 (d, J=1.71 Hz, 1H), 7.81 (d, J=8.68 Hz, 1H), 6.64 (d, J=8.68 Hz, 1H), 4.20-4.31 (m, 1H), 4.09-4.20 (m, 1H), 4.05 (d, J=11.74 Hz, 1H), 3.79-3.90 (m, 2H), 3.56 (d, J=11.62 Hz, 2H), 3.40 (d, J=12.10 Hz, 1H), 3.25-3.34 (m, 2H), 3.01-3.15 (m, 6H), 2.38 (m, 1H), 2.20 (d, J=10.88 Hz, 2H), 1.74-1.97 (m, 2H), 0.62-0.86 (m, 4H).

### Example 50

### 8-[7-(3,8-Diazabicyclo[4.2.0]octan-8-ylmethyl)-5-azaspiro[2.4]heptan-5-yl]quinoxaline-5-carbonitrile

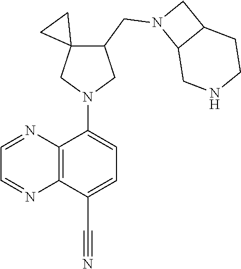 [SMILES: N#Cc1ccc(N2CC(CN3CC4CCNCC43)C3(CC3)C2)c2nccnc12]

The title compound was prepared in analogy to the preparation of Example 3 by using tert-butyl 3,8-diazabicyclo[4.2.0]octane-3-carboxylate instead of tert-butyl piperazine-1-carboxylate (compound 3g). Example 50 (18 mg) was obtained as a yellow solid. MS: calc'd 375 (MH⁺), measured 375 (MH⁺). ¹H NMR (400 MHz, METHANOL-d₄) δ=8.69 (dd, J=1.83, 5.87 Hz, 1H), 8.59 (dd, J=1.71, 7.34 Hz, 1H), 7.79 (t, J=8.68 Hz, 1H), 6.62 (d, J=8.80 Hz, 1H), 4.31-4.54 (m, 1H), 3.85-4.22 (m, 4H), 3.55-3.84 (m, 3H), 3.24-3.46 (m, 3H), 2.95-3.15 (m, 1H), 2.75-2.91 (m, 2H), 2.19-2.33 (m, 1H), 1.91-2.12 (m, 2H), 0.75-0.87 (m, 1H), 0.58-0.74 (m, 3H).

### Example 51

### 8-[(3S,4R)-3-[(4-amino-1-piperidyl)methyl]-4-(trifluoromethyl)pyrrolidin-1-yl]quinoxaline-5-carbonitrile

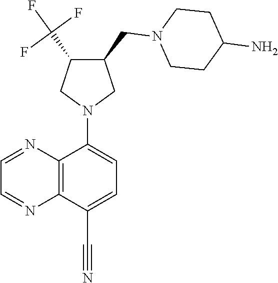 [SMILES: N#Cc1ccc(N2C[C@H](CN3CCC(N)CC3)[C@@H](C(F)(F)F)C2)c2nccnc12]

The title compound was prepared in analogy to the preparation of Example 1 by using tert-butyl N-(4-piperidyl)carbamate instead of tert-butyl 3,9-diazaspiro[5.5]undecane-3-carboxylate (compound 1e). Example 51 (16 mg) was obtained as a yellow solid. MS: calc'd 405 (MH⁺), measured 405 (MH⁺). ¹H NMR (400 MHz, METHANOL-d₄) δ=8.88 (d, J=1.8 Hz, 1H), 8.80 (d, J=1.7 Hz, 1H), 8.00 (d, J=8.6 Hz, 1H), 6.84 (d, J=8.7 Hz, 1H), 4.45-4.21 (m, 3H), 3.89 (dd, J=6.4, 11.4 Hz, 1H), 3.81-3.58 (m, 2H), 3.50-3.36 (m, 2H), 3.20-2.86 (m, 5H), 2.24 (br d, J=11.7 Hz, 2H), 2.08-1.89 (m, 2H).

### Example 52

### 5-[(3S,4R)-3-[(4-piperazin-1-yl-1-piperidyl)methyl]-4-(trifluoromethyl)pyrrolidin-1-yl]quinoline-8-carbonitrile

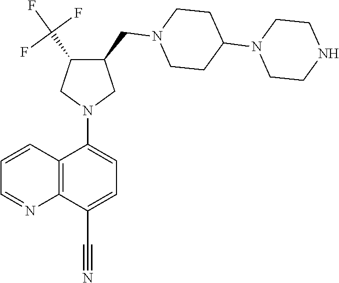 [SMILES: N#Cc1ccc(N2C[C@H](CN3CCC(N4CCNCC4)CC3)[C@@H](C(F)(F)F)C2)c2cccnc12]

The title compound was prepared in analogy to the preparation of Example 1 by using tert-butyl 4-(4-piperidyl)piperazine-1-carboxylate and 5-bromoquinoline-8-carbonitrile instead of tert-butyl 3,9-diazaspiro[5.5]undecane-3-carboxylate (compound 1e) and bromoquinoxaline-5-carbonitrile (compound 1a). Example 52 (10 mg) was obtained as a yellow solid. MS: calc'd 473 (MH⁺), measured 473 (MH⁺). ¹H NMR (400 MHz, METHANOL-d₄) δ=8.93 (dd, J=1.5, 4.3 Hz, 1H), 8.69 (dd, J=1.5, 8.7 Hz, 1H), 8.03 (d, J=8.2 Hz, 1H), 7.57 (dd, J=4.3, 8.7 Hz, 1H), 7.05 (d, J=8.3 Hz, 1H), 3.99 (dd, J=7.0, 9.8 Hz, 1H), 3.89-3.64 (m, 6H), 3.59-3.42 (m, 3H), 3.28-3.09 (m 6H), 3.06-2.88 (m 5H), 2.29-1.96 (m 4H).

### Example 53

### 5-[(3S,4R)-3-[(6-Amino-2-azaspiro[3.3]heptan-2-yl)methyl]-4-(trifluoromethyl)pyrrolidin-1-yl]quinoline-8-carbonitrile

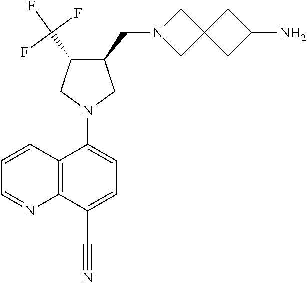 [SMILES: N#Cc1ccc(N2C[C@H](CN3CC4(CC(N)C4)C3)[C@@H](C(F)(F)F)C2)c2cccnc12]

The title compound was prepared in analogy to the preparation of Example 1 by using tert-butyl N-(2-azaspiro[3.3]heptan-6-yl)carbamate and 5-bromoquinoline-8-carbonitrile instead of tert-butyl 3,9-diazaspiro[5.5]undecane-3-carboxylate (compound 1e) and bromoquinoxaline-5-carbonitrile (compound 1a). Example 53 (6 mg) was obtained as a yellow solid. MS: calc'd 416 (MH⁺), measured 416 (MH⁺). ¹H NMR (400 MHz, METHANOL-d₄) δ=8.95 (dd, J=1.6, 4.3 Hz, 1H), 8.66 (dd, J=1.6, 8.7 Hz, 1H), 8.08 (d, J=8.2 Hz, 1H), 7.59 (dd, J=4.2, 8.7 Hz, 1H), 7.09 (d, J=8.3 Hz, 1H), 4.45-4.18 (m, 4H), 3.89-3.64 (m, 4H), 3.62-3.36 (m, 3H), 3.26-3.08 (m, 1H), 2.95-2.67 (m, 3H), 2.55-2.39 (m, 2H).

### Example 54

### 5-[(3S,4R)-3-(2,8-Diazaspiro[4.5]decan-8-ylmethyl)-4-(trifluoromethyl)pyrrolidin-1-yl]quinoline-8-carbonitrile

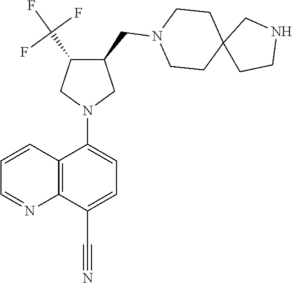 [SMILES: N#Cc1ccc(N2C[C@H](CN3CCC4(CCNC4)CC3)[C@@H](C(F)(F)F)C2)c2cccnc12]

The title compound was prepared in analogy to the preparation of Example 1 by using tert-butyl 2,8-diazaspiro[4.5]decane-2-carboxylate and 5-bromoquinoline-8-carbonitrile instead of tert-butyl 3,9-diazaspiro[5.5]undecane-3-carboxylate (compound 1e) and bromoquinoxaline-5-carbonitrile (compound 1a). Example 54 (20 mg) was obtained as a yellow solid. MS: calc'd 444 (MH⁺), measured 444 (MH⁺). ¹H NMR (400 MHz, METHANOL-d₄) δ=8.93 (dd, J=1.5, 4.2 Hz, 1H), 8.68 (dd, J=1.6, 8.7 Hz, 1H), 8.03 (d, J=8.2 Hz, 1H), 7.57 (dd, J=4.2, 8.7 Hz, 1H), 7.05 (d, J=8.3 Hz, 1H), 4.00 (dd, J=7.0, 9.8 Hz, 1H), 3.88-3.79 (m, 1H), 3.73 (dd, J=6.2, 10.5 Hz, 2H), 3.65-3.37 (m, 6H), 3.27-3.03 (m, 6H), 2.19-1.87 (m, 6H).

### Example 55

### 5-[(3S,4R)-3-(2,9-Diazaspiro[5.5]undecan-9-ylmethyl)-4-(trifluoromethyl)pyrrolidin-1-yl]quinoline-8-carbonitrile

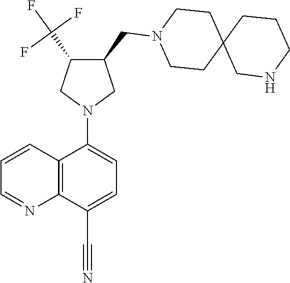 [SMILES: N#Cc1ccc(N2C[C@H](CN3CCC4(CCCNC4)CC3)[C@@H](C(F)(F)F)C2)c2cccnc12]

The title compound was prepared in analogy to the preparation of Example 1 by using tert-butyl 2,9-diazaspiro[5.5]undecane-2-carboxylate and 5-bromoquinoline-8-carbonitrile instead of tert-butyl 3,9-diazaspiro[5.5]undecane-3-carboxylate (compound 1e) and bromoquinoxaline-5-carbonitrile (compound 1a). Example 55 (11 mg) was obtained as a yellow solid. MS: calc'd 458 (MH⁺), measured 458 (MH⁺). ¹H NMR (400 MHz, METHANOL-d₄) 6=8.92 (dd, J=1.6, 4.3 Hz, 1H), 8.68 (dd, J=1.6, 8.7 Hz, 1H), 8.02 (d, J=8.2 Hz, 1H), 7.56 (dd, J=4.2, 8.7 Hz, 1H), 7.04 (d, J=8.3 Hz, 1H), 4.00 (dd, J=6.9, 10.0 Hz, 1H), 3.88-3.78 (m, 1H), 3.73 (dd, J=6.2, 10.5 Hz, 2H), 3.64-3.41 (m, 5H), 3.26-2.95 (m, 8H), 2.12-1.75 (m, 7H).

### Example 56

### 5-[(3S,4R)-3-(2,8-Diazaspiro[4.5]decan-2-ylmethyl)-4-(trifluoromethyl)pyrrolidin-1-yl]quinoline-8-carbonitrile

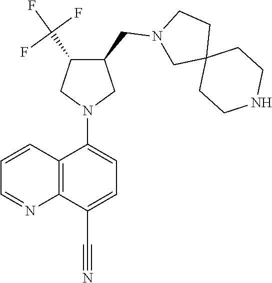 [SMILES: N#Cc1ccc(N2C[C@H](CN3CCC4(CCNCC4)C3)[C@@H](C(F)(F)F)C2)c2cccnc12]

The title compound was prepared in analogy to the preparation of Example 1 by using tert-butyl 2,8-diazaspiro[4.5]decane-8-carboxylate and 5-bromoquinoline-8-carbonitrile instead of tert-butyl 3,9-diazaspiro[5.5]undecane-3-carboxylate (compound 1e) and bromoquinoxaline-5-carbonitrile (compound 1a). Example 56 (10 mg) was obtained as a yellow solid. MS: calc'd 444 (MH⁺), measured 444 (MH⁺). ¹H NMR (400 MHz, METHANOL-d₄) δ=8.93 (dd, J=1.6, 4.3 Hz, 1H), 8.66 (dd, J=1.5, 8.7 Hz, 1H), 8.03 (d, J=8.2 Hz, 1H), 7.56 (dd, J=4.3, 8.7 Hz, 1H), 7.04 (d, J=8.3 Hz, 1H), 3.99 (dd, J=7.2, 9.8 Hz, 1H), 3.86-3.51 (m, 8H), 3.27-3.04 (m, 7H), 2.19-1.85 (m, 6H).

### Example 57

### 5-[(3S,4R)-3-(2,9-Diazaspiro[5.5]undecan-2-ylmethyl)-4-(trifluoromethyl)pyrrolidin-1-yl]quinoline-8-carbonitrile

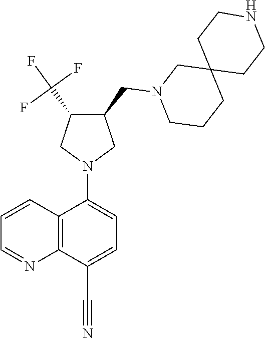 [SMILES: N#Cc1ccc(N2C[C@H](CN3CCCC4(CCNCC4)C3)[C@@H](C(F)(F)F)C2)c2cccnc12]

The title compound was prepared in analogy to the preparation of Example 1 by using tert-butyl 2,9-diazaspiro[5.5]undecane-9-carboxylate and 5-bromoquinoline-8-carbonitrile instead of tert-butyl 3,9-diazaspiro[5.5]undecane-3-carboxylate (compound 1e) and bromoquinoxaline-5-carbonitrile (compound 1a). Example 57 (25 mg) was obtained as a yellow solid. MS: calc'd 458 (MH⁺), measured 458 (MH⁺). ¹H NMR (400 MHz, METHANOL-d₄) 6=8.93 (dd, J=1.3, 4.2 Hz, 1H), 8.66 (dd, J=1.3, 8.7 Hz, 1H), 8.01 (d, J=8.2 Hz, 1H), 7.57 (dd, J=4.3, 8.7 Hz, 1H), 7.03 (d, J=8.2 Hz, 1H), 3.97 (br dd, J=6.5, 9.8 Hz, 1H), 3.90-3.44 (m, 8H), 3.17-2.87 (m, 8H), 2.19-1.60 (m, 7H).

### Example 58

### 8-[7-[(4-Morpholino-1-piperidyl)methyl]-5-azaspiro[2.4]heptan-5-yl]quinoxaline-5-carbonitrile

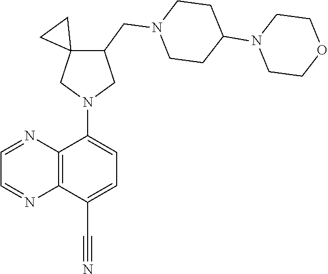 [SMILES: N#Cc1ccc(N2CC(CN3CCC(N4CCOCC4)CC3)C3(CC3)C2)c2nccnc12]

The title compound was prepared in analogy to the preparation of Example 3 by using 4-(4-piperidyl)morpholine instead of tert-butyl piperazine-1-carboxylate (compound 3g). Example 58 (14 mg) was obtained as a yellow solid. MS: calc'd 433 (MH⁺), measured 433 (MH⁺). ¹H NMR (400 MHz, METHANOL-d₄) δ=8.83 (d, J=1.71 Hz, 1H), 8.73 (d, J=1.83 Hz, 1H), 7.93 (d, J=8.68 Hz, 1H), 6.75 (d, J=8.68 Hz, 1H), 4.32-4.42 (m, 1H), 4.22-4.30 (m, 1H), 4.17 (d, J=11.74 Hz, 1H), 3.96 (m, 5H), 3.82 (d, J=13.08 Hz, 1H), 3.67 (d, J=11.62 Hz, 1H), 3.49-3.59 (m, 2H), 3.36-3.49 (m, 4H), 3.14-3.25 (m, 2H), 3.08 (t, J=13.02 Hz, 1H), 2.37-2.55 (m, 3H), 2.08-2.24 (m, 2H), 0.74-0.98 (m, 4H).

### Example 59

### 8-[(3S,4R)-3-(Piperazin-1-ylmethyl)-4-(trifluoromethyl)pyrrolidin-1-yl]quinoxaline-5-carbonitrile

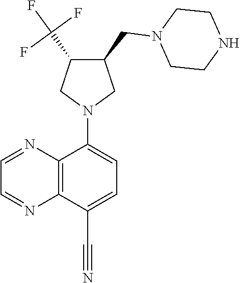 [SMILES: N#Cc1ccc(N2C[C@H](CN3CCNCC3)[C@@H](C(F)(F)F)C2)c2nccnc12]

The title compound was prepared in analogy to the preparation of Example 1 by using tert-butyl piperazine-1-carboxylate instead of tert-butyl 3,9-diazaspiro[5.5]undecane-3-carboxylate (compound 1e). Example 59 (15 mg) was obtained as a yellow solid. MS: calc'd 391 (MH⁺), measured 391 (MH⁺). ¹H NMR (400 MHz, METHANOL-d₄) δ=8.86 (d, J=1.7 Hz, 1H), 8.79 (d, J=1.7 Hz, 1H), 8.00 (d, J=8.6 Hz, 1H), 6.83 (d, J=8.7 Hz, 1H), 4.44 (dd, J=8.3, 12.8 Hz, 1H), 4.26 (dd, J=5.2, 12.8 Hz, 1H), 4.12 (dd, J=7.5, 11.4 Hz, 1H), 3.81 (dd, J=4.8, 11.6 Hz, 1H), 3.53-3.44 (m, 1H), 3.25-3.14 (m, 4H), 2.95-2.54 (m, 7H).

### Example 60

### 8-[(3S,4R)-3-[(4-Pyrrolidin-1-yl-1-piperidyl)methyl]-4-(trifluoromethyl)pyrrolidin-1-yl]quinoxaline-5-carbonitrile

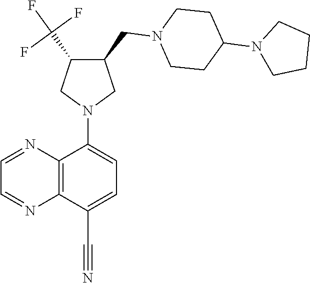 [SMILES: N#Cc1ccc(N2C[C@H](CN3CCC(N4CCCC4)CC3)[C@@H](C(F)(F)F)C2)c2nccnc12]

The title compound was prepared in analogy to the preparation of Example 1 by using 4-pyrrolidin-1-ylpiperidine instead of tert-butyl 3,9-diazaspiro[5.5]undecane-3-carboxylate (compound 1e). Example 60 (17 mg) was obtained as a yellow solid. MS: calc'd 459 (MH⁺), measured 459 (MH⁺). ¹H NMR (400 MHz, METHANOL-d₄) δ=8.88 (d, J=1.7 Hz, 1H), 8.80 (d, J=1.7 Hz, 1H), 8.01 (d, J=8.7 Hz, 1H), 6.85 (d, J=8.7 Hz, 1H), 4.47-4.22 (m, 3H), 3.88 (dd, J=5.9, 11.6 Hz, 1H), 3.77-3.47 (m, 4H), 3.27-2.97 (m, 7H), 2.84 (br d, J=15.0 Hz, 2H), 2.37 (br d, J=12.6 Hz, 2H), 2.25-1.87 (m 6H).

### Example 61

### 8-[(3S,4R)-3-[[4-(1-Piperidyl)-1-piperidyl]methyl]-4-(trifluoromethyl)pyrrolidin-1-yl]quinoxaline-5-carbonitrile

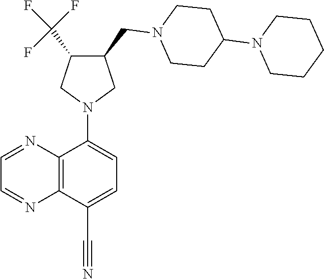 [SMILES: N#Cc1ccc(N2C[C@H](CN3CCC(N4CCCCC4)CC3)[C@@H](C(F)(F)F)C2)c2nccnc12]

The title compound was prepared in analogy to the preparation of Example 1 by using 1-(4-piperidyl)piperidine instead of tert-butyl 3,9-diazaspiro[5.5]undecane-3-carboxylate (compound 1e). Example 61 (26 mg) was obtained as a yellow solid. MS: calc'd 473 (MH⁺), measured 473 (MH⁺). ¹H NMR (400 MHz, METHANOL-d₄) δ=8.86 (d, J=1.7 Hz, 1H), 8.78 (d, J=1.6 Hz, 1H), 7.95 (d, J=8.6 Hz, 1H), 6.80 (d, J=8.7 Hz, 1H), 4.47-4.22 (m, 3H), 4.03-3.74 (m, 3H), 3.63-3.36 (m, 5H), 3.24-2.93 (m, 6H), 2.40 (br d, J=11.1 Hz, 2H), 2.31-2.14 (m, 2H), 2.07-1.64 (m, 6H).

### Example 62

### 8-[(3S,4R)-3-[[4-(Azepan-1-yl)-1-piperidyl]methyl]-4-(trifluoromethyl)pyrrolidin-1-yl]quinoxaline-5-carbonitrile

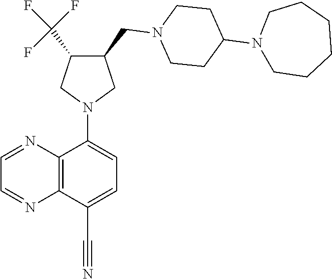 [SMILES: N#Cc1ccc(N2C[C@H](CN3CCC(N4CCCCCC4)CC3)[C@@H](C(F)(F)F)C2)c2nccnc12]

The title compound was prepared in analogy to the preparation of Example 1 by using 1-(4-piperidyl)azepane instead of tert-butyl 3,9-diazaspiro[5.5]undecane-3-carboxylate (compound 1e). Example 62 (20 mg) was obtained as a yellow solid. MS: calc'd 487 (MH⁺), measured 487 (MH⁺). ¹H NMR (400 MHz, METHANOL-d₄) δ=8.87 (d, J=1.7 Hz, 1H), 8.80 (d, J=1.6 Hz, 1H), 8.00 (d, J=8.6 Hz, 1H), 6.84 (d, J=8.7 Hz, 1H), 4.48-4.22 (m, 3H), 3.89 (dd, J=5.8, 11.6 Hz, 1H), 3.76 (br d, J=9.8 Hz, 1H), 3.69-3.39 (m, 4H), 3.25-2.76 (m, 8H), 2.30 (br d, J=13.4 Hz, 2H), 2.20-1.81 (m, 6H), 1.75 (br s, 4H).

### Example 63

### 8-[(3S,4R)-3-[(3-amino-8-azabicyclo[3.2.1]octan-8-yl)methyl]-4-(trifluoromethyl)pyrrolidin-1-yl]quinoxaline-5-carbonitrile

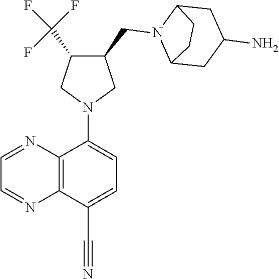 [SMILES: N#Cc1ccc(N2C[C@H](CN3C4CCC3CC(N)C4)[C@@H](C(F)(F)F)C2)c2nccnc12]

The title compound was prepared in analogy to the preparation of Example 1 by using tert-butyl N-(8-azabicyclo[3.2.1]octan-3-yl)carbamate instead of tert-butyl 3,9-diazaspiro[5.5]undecane-3-carboxylate (compound 1e). Example 63 (9 mg) was obtained as a yellow solid. MS: calc'd 431 (MH⁺), measured 431 (MH⁺). ¹H NMR (400 MHz, METHANOL-d₄) δ=8.90-8.84 (m, 1H), 8.82-8.75 (m, 1H), 8.01-7.93 (m, 1H), 6.91-6.79 (m, 1H), 4.47-4.18 (m, 5H), 3.99 (dd, J=6.1, 11.4 Hz, 1H), 3.75 (tt, J=5.8, 11.6 Hz, 1H), 3.51-3.33 (m, 3H), 3.23-3.03 (m, 1H), 2.54-2.00 (m, 8H).

### Example 64

### 8-[(3S,4R)-3-[(4-amino-3,3-difluoro-1-piperidyl)methyl]-4-(trifluoromethyl)pyrrolidin-1-yl]quinoxaline-5-carbonitrile

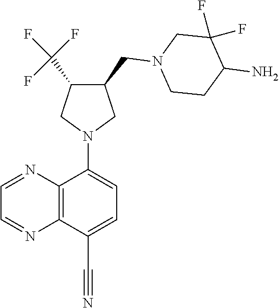 [SMILES: N#Cc1ccc(N2C[C@H](CN3CCC(N)C(F)(F)C3)[C@@H](C(F)(F)F)C2)c2nccnc12]

The title compound was prepared in analogy to the preparation of Example 1 by using tert-butyl N-(3,3-difluoro-4-piperidyl)carbamate instead of tert-butyl 3,9-diazaspiro[5.5]undecane-3-carboxylate (compound 1e). Example 64 (10 mg) was obtained as a yellow solid. MS: calc'd 441 (MH⁺), measured 441 (MH⁺). ¹H NMR (400 MHz, METHANOL-d₄) δ=8.86 (d, J=1.8 Hz, 1H), 8.79 (d, J=1.6 Hz, 1H), 8.00 (d, J=8.7 Hz, 1H), 6.83 (d, J=8.7 Hz, 1H), 4.44 (br dd, J=8.8, 12.2 Hz, 1H), 4.25 (dd, J=4.9, 13.0 Hz, 1H), 4.10 (td, J=5.9, 11.9 Hz, 1H), 3.88-3.55 (m, 2H), 3.25-3.03 (m, 3H), 2.94-2.81 (m, 1H), 2.72-2.49 (m, 3H), 2.37 (br t, J=11.7 Hz, 1H), 2.18-1.80 (m, 2H).

### Example 65

### 8-[7-(1,9-Diazaspiro[5.5]undecan-9-ylmethyl)-5-azaspiro[2.4]heptan-5-yl]quinoxaline-5-carbonitrile

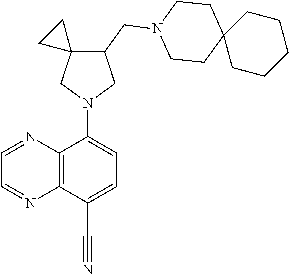 [SMILES: N#Cc1ccc(N2CC(CN3CCC4(CCCCC4)CC3)C3(CC3)C2)c2nccnc12]

The title compound was prepared in analogy to the preparation of Example 3 by using tert-butyl 1,9-diazaspiro[5.5]undecane-1-carboxylate instead of tert-butyl piperazine-1-carboxylate (compound 3g). Example 65 (28 mg) was obtained as a yellow solid. MS: calc'd 417 (MH⁺), measured 417 (MH⁺). ¹H NMR (400 MHz, METHANOL-d₄) δ=8.85 (d, J=1.71 Hz, 1H), 8.76 (d, J=1.71 Hz, 1H), 7.98 (d, J=8.68 Hz, 1H), 6.79 (d, J=8.80 Hz, 1H), 4.38 (dd, J=5.93, 11.92 Hz, 1H), 4.25 (d, J=10.03 Hz, 1H), 4.18 (d, J=11.74 Hz, 1H), 3.52-3.80 (m, 3H), 3.35-3.43 (m, 2H), 3.12-3.28 (m, 4H), 2.46 (m, 1H), 2.33 (m, 2H), 2.16 (m, 2H), 1.91-2.07 (m, 2H), 1.70-1.88 (m, 4H), 0.74-0.96 (m, 4H).

### Example 66

### 8-[7-(3,7-Diazabicyclo[4.2.0]octan-3-ylmethyl)-5-azaspiro[2.4]heptan-5-yl]quinoxaline-5-carbonitrile

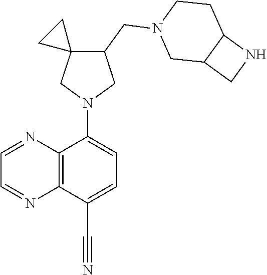 [SMILES: N#Cc1ccc(N2CC(CN3CCC4NCC4C3)C3(CC3)C2)c2nccnc12]

The title compound was prepared in analogy to the preparation of Example 3 by using tert-butyl 3,7-diazabicyclo[4.2.0]octane-7-carboxylate instead of tert-butyl piperazine-1-carboxylate (compound 3g). Example 66 (12 mg) was obtained as a yellow solid. MS: calc'd 375 (MH⁺), measured 375 (MH⁺). ¹H NMR (400 MHz, METHANOL-d₄) δ=8.85 (d, J=1.83 Hz, 1H), 8.76 (d, J=1.71 Hz, 1H), 7.98 (d, J=8.68 Hz, 1H), 6.79 (d, J=8.68 Hz, 1H), 4.66 (m, 1H), 4.30-4.40 (m, 1H), 3.97-4.29 (m, 4H), 3.71 (dd, J=2.51, 11.43 Hz, 1H), 3.22-3.61 (m, 6H), 3.07-3.18 (m, 1H), 2.45 (m, 3H), 0.87-0.96 (m, 1H), 0.75-0.87 (m, 3H).

### Example 67

### 8-[7-(1-Oxa-4,9-diazaspiro[5.5]undecan-4-ylmethyl)-5-azaspiro[2.4]heptan-5-yl]quinoxaline-5-carbonitrile

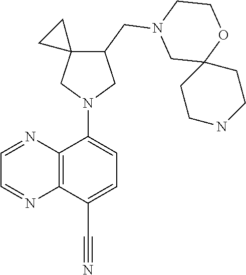 [SMILES: N#Cc1ccc(N2CC(CN3CCOC4(CCNCC4)C3)C3(CC3)C2)c2nccnc12]

The title compound was prepared in analogy to the preparation of Example 3 by using tert-butyl 1-oxa-4,9-diazaspiro[5.5]undecane-9-carboxylate instead of tert-butyl piperazine-1-carboxylate (compound 3g). Example 67 (25 mg) was obtained as a yellow solid. MS: calc'd 419 (MH⁺), measured 419 (MH⁺). ¹H NMR (400 MHz, METHANOL-d₄) δ=8.84 (d, J=1.83 Hz, 1H), 8.75 (d, J=1.83 Hz, 1H), 7.96 (d, J=8.68 Hz, 1H), 6.77 (d, J=8.80 Hz, 1H), 4.26-4.37 (m, 1H), 4.11-4.26 (m, 2H), 3.96 (t, J=4.71 Hz, 2H), 3.72 (d, J=11.62 Hz, 1H), 2.94-3.29 (m, 10H), 2.19-2.56 (m, 3H), 1.70-1.88 (m, 2H), 0.74-0.97 (m, 4H).

### Example 68

### 8-[7-[(4-Amino-1-piperidyl)methyl]-5-azaspiro[2.4]heptan-5-yl]quinoxaline-5-carbonitrile

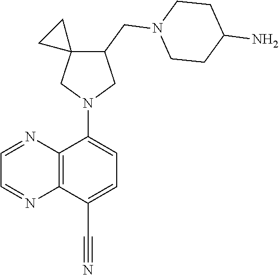 [SMILES: N#Cc1ccc(N2CC(CN3CCC(N)CC3)C3(CC3)C2)c2nccnc12]

The title compound was prepared in analogy to the preparation of Example 3 by using tert-butyl N-(4-piperidyl)carbamate instead of tert-butyl piperazine-1-carboxylate (compound 3g). Example 68 (17 mg) was obtained as a yellow solid. MS: calc'd 363 (MH⁺), measured 363 (MH⁺). ¹H NMR (400 MHz, METHANOL-d₄) δ=8.84 (d, J=1.71 Hz, 1H), 8.76 (d, J=1.71 Hz, 1H), 7.98 (d, J=8.68 Hz, 1H), 6.77 (d, J=8.68 Hz, 1H), 4.18-4.27 (m, 1H), 4.03-4.17 (m, 2H), 3.77 (d, J=11.62 Hz, 1H), 3.10-3.29 (m, 3H), 2.67-2.80 (m, 1H), 2.58 (d, J=12.47 Hz, 1H), 2.45 (s, 1H), 2.31 (d, J=3.67 Hz, 2H), 2.06 (t, J=12.53 Hz, 2H), 1.74 (d, J=12.10 Hz, 2H), 0.80-0.89 (m, 1H), 0.65-0.80 (m, 3H).

SFC-HPLC (40% CO₂/0.5% NH₃ in methanol as eluent on Daicel AD-H Column) separation gave 2 isomers: Example 68A (5 mg) and Example 68B (2 mg).

Example 68A: MS: calc'd 363 (MH⁺), measured 363 (MH⁺). ¹H NMR (400 MHz, METHANOL-d₄) δ=8.85 (d, J=1.83 Hz, 1H), 8.76 (d, J=1.71 Hz, 1H), 7.97 (d, J=8.68 Hz, 1H), 6.78 (d, J=8.80 Hz, 1H), 4.33-4.41 (m, 1H), 4.21-4.29 (m, 1H), 4.18 (d, J=11.74 Hz, 1H), 3.85 (m, 1H), 3.62-3.76 (m, 2H), 3.36-3.52 (m, 2H), 3.02-3.24 (m, 3H), 2.41-2.52 (m, 1H), 2.28 (t, J=13.57 Hz, 2H), 1.92-2.11 (m 2H), 0.77-0.94 (m 4H).

Example 68B: MS: calc'd 363 (MH⁺), measured 363 (MH⁺). ¹H NMR (400 MHz, METHANOL-d₄) δ=8.84 (d, J=1.83 Hz, 1H), 8.76 (d, J=1.59 Hz, 1H), 7.97 (d, J=8.68 Hz, 1H), 6.78 (d, J=8.80 Hz, 1H), 4.32-4.41 (m, 1H), 4.22-4.31 (m, 1H), 4.17 (d, J=11.74 Hz, 1H), 3.84 (m, 1H), 3.62-3.76 (m, 2H), 3.35-3.53 (m, 2H), 3.00-3.25 (m, 3H), 2.39-2.54 (m, 1H), 2.27 (t, J=14.24 Hz, 2H), 1.92-2.12 (m, 2H), 0.73-0.97 (m, 4H).

### Example 69

### 1-[[(3S,4R)-1-(S-cyanoquinoxalin-5-yl)-4-(trifluoromethyl)pyrrolidin-3-yl]methyl]piperidine-3-carboxamide

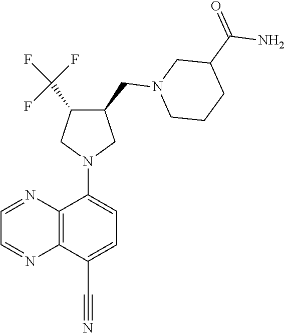 [SMILES: N#Cc1ccc(N2C[C@H](CN3CCCC(C(N)=O)C3)[C@@H](C(F)(F)F)C2)c2nccnc12]

The title compound was prepared in analogy to the preparation of Example 1 by using piperidine-3-carboxamide instead of tert-butyl 3,9-diazaspiro[5.5]undecane-3-carboxylate (compound 1e). Example 69 (11 mg) was obtained as a yellow solid. MS: calc'd 433 (MH⁺), measured 433 (MH⁺). ¹H NMR (400 MHz, METHANOL-d₄) δ=8.96-8.74 (m, 2H), 8.03 (d, J=8.6 Hz, 1H), 6.89 (br s, 1H), 4.54-4.18 (m, 4H), 4.11-3.76 (m, 3H), 3.48 (br d, J=1.6 Hz, 3H), 3.19-2.86 (m, 2H), 2.35-1.80 (m, 4H).

### Example 70

### 8-[(3S,4R)-3-(1-oxa-4,9-diazaspiro[5.5]undecan-9-ylmethyl)-4-(trifluoromethyl)pyrrolidin-1-yl]quinoxaline-5-carbonitrile

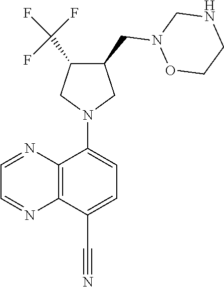 [SMILES: N#Cc1ccc(N2C[C@H](CN3CNCCO3)[C@@H](C(F)(F)F)C2)c2nccnc12]

The title compound was prepared in analogy to the preparation of Example 1 by using tert-butyl 1-oxa-4,9-diazaspiro[5.5]undecane-4-carboxylate instead of tert-butyl 3,9-diazaspiro[5.5]undecane-3-carboxylate (compound 1e). Example 70 (13 mg) was obtained as a yellow solid. MS: calc'd 461 (MH⁺), measured 461 (MH⁺). ¹H NMR (400 MHz, METHANOL-d₄) δ=8.94-8.70 (m, 2H), 8.10-7.85 (m, 1H), 6.92-6.74 (m, 1H), 4.48-4.21 (m, 3H), 4.05-3.82 (m, 3H), 3.73-3.39 (m, 5H), 3.21-2.92 (m, 7H), 2.46-1.89 (m, 4H).

### Example 71

### 8-[(3S,4R)-3-[(4-piperidylamino)methyl]-4-(trifluoromethyl)pyrrolidin-1-yl]quinoxaline-5-carbonitrile

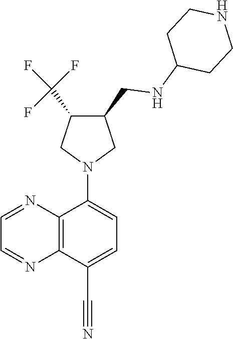 [SMILES: N#Cc1ccc(N2C[C@H](CNC3CCNCC3)[C@@H](C(F)(F)F)C2)c2nccnc12]

The title compound was prepared in analogy to the preparation of Example 1 by using tert-butyl 4-aminopiperidine-1-carboxylate instead of tert-butyl 3,9-diazaspiro[5.5]undecane-3-carboxylate (compound 1e). Example 71 (9 mg) was obtained as a yellow solid. MS: calc'd 405 (MH⁺), measured 405 (MH⁺). ¹H NMR (400 MHz, METHANOL-d₄) δ=9.00-8.67 (m, 2H), 7.98 (d, J=8.6 Hz, 1H), 6.83 (d, J=8.7 Hz, 1H), 4.49-4.15 (m, 3H), 4.05-3.76 (m, 1H), 3.69-3.38 (m 5H), 3.22-2.82 (m, 4H), 2.41 (br t, J=12.7 Hz, 2H), 2.12-1.76 (m 2H).

### Example 72

### 8-[(3S,4R)-3-[[(3S,4R)-4-amino-3-methyl-1-piperidyl]methyl]-4-(trifluoromethyl)pyrrolidin-1-yl]quinoxaline-5-carbonitrile

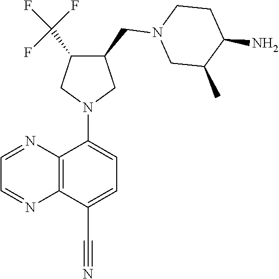 [SMILES: C[C@H]1CN(C[C@H]2CN(c3ccc(C#N)c4nccnc34)C[C@@H]2C(F)(F)F)CC[C@H]1N]

The title compound was prepared in analogy to the preparation of Example 1 by using tert-butyl N-[(3S,4R)-3-methyl-4-piperidyl]carbamate instead of tert-butyl 3,9-diazaspiro[5.5]undecane-3-carboxylate (compound 1e). Example 72 (37 mg) was obtained as a yellow solid. MS: calc'd 419 (MH⁺), measured 419 (MH⁺). ¹H NMR (400 MHz, METHANOL-d₄) δ=8.93-8.73 (m, 2H), 8.00 (d, J=8.6 Hz, 1H), 6.84 (dd, J=1.9, 8.6 Hz, 1H), 4.48-4.19 (m, 3H), 3.88 (br d, J=11.1 Hz, 1H), 3.69-3.41 (m, 1H), 3.25-2.95 (m, 6H), 2.50-1.99 (m, 3H), 1.35-1.05 (m, 3H).

### Example 73

### 8-[(3S,4R)-3-[(8-amino-3-azabicyclo[3.2.1]octan-3-yl)methyl]-4-(trifluoromethyl)pyrrolidin-1-yl]quinoxaline-5-carbonitrile

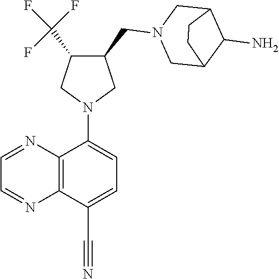 [SMILES: N#Cc1ccc(N2C[C@H](CN3CC4CCC(C3)C4N)[C@@H](C(F)(F)F)C2)c2nccnc12]

The title compound was prepared in analogy to the preparation of Example 1 by using tert-butyl N-(3-azabicyclo[3.2.1]octan-8-yl)carbamate instead of tert-butyl 3,9-diazaspiro[5.5]undecane-3-carboxylate (compound 1e). Example 73 (37 mg) was obtained as a yellow solid. MS: calc'd 431 (MH⁺), measured 431 (MH⁺). ¹H NMR (400 MHz, METHANOL-d₄) δ=8.99-8.64 (m, 2H), 7.97 (d, J=8.7 Hz, 1H), 6.81 (d, J=8.8 Hz, 1H), 4.47-4.16 (m, 3H), 3.87 (br d, J=10.1 Hz, 1H), 3.25-2.79 (m, 8H), 2.42 (br s, 2H), 1.96 (br s, 4H).

### Example 74

### 8-[(3S,4R)-3-[[4-(aminomethyl)-1-piperidyl]methyl]-4-(trifluoromethyl)pyrrolidin-1-yl]quinoxaline-5-carbonitrile

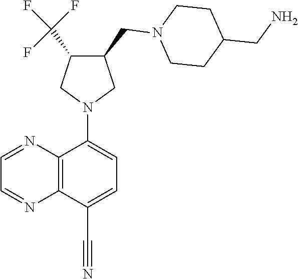 [SMILES: N#Cc1ccc(N2C[C@H](CN3CCC(CN)CC3)[C@@H](C(F)(F)F)C2)c2nccnc12]

The title compound was prepared in analogy to the preparation of Example 1 by using tert-butyl N-(4-piperidylmethyl)carbamate instead of tert-butyl 3,9-diazaspiro[5.5]undecane-3-carboxylate (compound 1e). Example 74 (22 mg) was obtained as a yellow solid. MS: calc'd 419 (MH⁺), measured 419 (MH⁺). ¹H NMR (400 MHz, METHANOL-d) δ=8.94-8.73 (m, 2H), 7.99 (br d, J=8.4 Hz, 1H), 6.84 (br d, J=8.4 Hz, 1H), 4.48-4.21 (m, 3H), 4.04-3.62 (m, 3H), 3.47 (br d, J=6.8 Hz, 2H), 3.22-2.72 (m, 6H), 2.20-1.93 (m, 3H), 1.71 (br d, J=12.7 Hz, 2H).

### Example 75

### 8-[(3S,4R)-3-[[3-(aminomethyl)-3-methyl-1-piperidyl]methyl]-4-(trifluoromethyl)pyrrolidin-1-yl]quinoxaline-5-carbonitrile

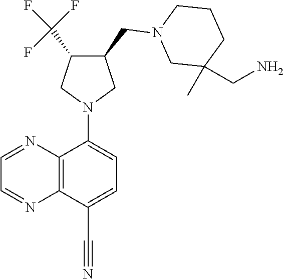 [SMILES: CC1(CN)CCCN(C[C@H]2CN(c3ccc(C#N)c4nccnc34)C[C@@H]2C(F)(F)F)C1]

The title compound was prepared in analogy to the preparation of Example 1 by using tert-butyl N-[(3-methyl-3-piperidyl)methyl]carbamate instead of tert-butyl 3,9-diazaspiro[5.5]undecane-3-carboxylate (compound 1e). Example 75 (22 mg) was obtained as a yellow solid. MS: calc'd 433 (MH⁺), measured 433 (MH⁺). ¹H NMR (400 MHz, METHANOL-d₄) δ=8.84 (d, J=1.8 Hz, 1H), 8.76 (s, 1H), 7.91 (dd, J=2.0, 8.6 Hz, 1H), 6.77 (dd, J=2.0, 8.7 Hz, 1H), 4.45-4.19 (m, 3H), 3.88 (td, J=6.0, 11.5 Hz, 1H), 3.46-3.34 (m, 2H), 3.25-2.90 (m, 8H), 2.07-1.54 (m, 4H), 1.33-1.10 (m, 3H).

### Example 76

### 8-[(3S,4R)-3-(2,3,3a,4,6,6a-hexahydro-1H-pyrrolo[3,4-c]pyrrol-5-ylmethyl)-4-(trifluoromethyl)pyrrolidin-1-yl]quinoxaline-5-carbonitrile

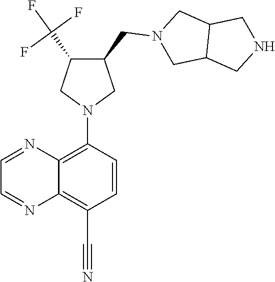 [SMILES: N#Cc1ccc(N2C[C@H](CN3CC4CNCC4C3)[C@@H](C(F)(F)F)C2)c2nccnc12]

The title compound was prepared in analogy to the preparation of Example 1 by using tert-butyl 2,3,3a,4,6,6a-hexahydro-1H-pyrrolo[3,4-c]pyrrole-5-carboxylate instead of tert-butyl 3,9-diazaspiro[5.5]undecane-3-carboxylate (compound 1e). Example 76 (21 mg) was obtained as a yellow solid. MS: calc'd 417 (MH⁺), measured 417 (MH⁺). ¹H NMR (400 MHz, METHANOL-d₄) δ=8.94-8.73 (m, 2H), 7.99 (d, J=8.6 Hz, 1H), 6.84 (d, J=8.6 Hz, 1H), 4.54-4.18 (m, 3H), 3.87 (dd, J=5.9, 11.4 Hz, 1H), 3.62-3.42 (m, 2H), 3.35-3.30 (m, 5H), 3.25-2.86 (m, 7H).

### Example 77

### 8-[(3S,4R)-3-(2,5-diazabicyclo[2.2.2]octan-2-ylmethyl)-4-(trifluoromethyl)pyrrolidin-1-yl]quinoxaline-5-carbonitrile

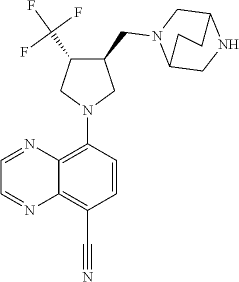 [SMILES: N#Cc1ccc(N2C[C@H](CN3CC4CCC3CN4)[C@@H](C(F)(F)F)C2)c2nccnc12]

The title compound was prepared in analogy to the preparation of Example 1 by using tert-butyl 2,5-diazabicyclo[2.2.2]octane-2-carboxylate instead of tert-butyl 3,9-diazaspiro[5.5]undecane-3-carboxylate (compound 1e). Example 77 (26 mg) was obtained as a yellow solid. MS: calc'd 417 (MH⁺), measured 417 (MH⁺). ¹H NMR (400 MHz, METHANOL-d₄) δ=8.94-8.68 (m, 2H), 8.00 (d, J=8.6 Hz, 1H), 6.83 (d, J=8.6 Hz, 1H), 4.44 (dd, J=8.4, 13.0 Hz, 1H), 4.31-4.01 (m, 2H), 3.92-3.75 (m, 1H), 3.62-3.42 (m, 2H), 3.21-2.73 (m, 8H), 2.35-1.58 (m, 4H).

### Example 78

### 8-[(3S,4R)-3-[[3-(aminomethyl)-3-fluoro-1-piperidyl]methyl]-4-(trifluoromethyl)pyrrolidin-1-yl]quinoxaline-5-carbonitrile

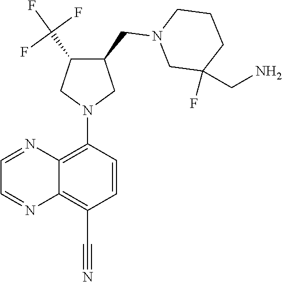 [SMILES: N#Cc1ccc(N2C[C@H](CN3CCCC(F)(CN)C3)[C@@H](C(F)(F)F)C2)c2nccnc12]

The title compound was prepared in analogy to the preparation of Example 1 by using tert-butyl N-[(3-fluoro-3-piperidyl)methyl]carbamate instead of tert-butyl 3,9-diazaspiro[5.5]undecane-3-carboxylate (compound 1e). Example 78 (15 mg) was obtained as a yellow solid. MS: calc'd 437 (MH⁺), measured 437 (MH⁺). ¹H NMR (400 MHz, METHANOL-d₄) δ=8.93-8.73 (m, 2H), 8.01 (dd, J=2.3, 8.6 Hz, 1H), 6.85 (d, J=8.7 Hz, 1H), 4.54-4.16 (m, 3H), 3.86 (br d, J=10.5 Hz, 1H), 3.26-2.70 (m, 10H), 2.22-1.59 (m, 4H).

### Example 79

### 8-[(3S,4R)-3-[[4-(2-aminoethyl)-1-piperidyl]methyl]-4-(trifluoromethyl)pyrrolidin-1-yl]quinoxaline-5-carbonitrile

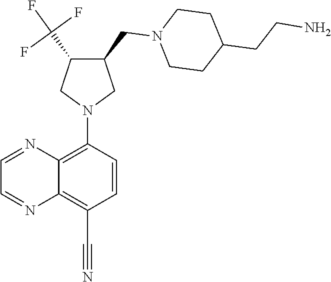 [SMILES: N#Cc1ccc(N2C[C@H](CN3CCC(CCN)CC3)[C@@H](C(F)(F)F)C2)c2nccnc12]

The title compound was prepared in analogy to the preparation of Example 1 by using tert-butyl N-[2-(4-piperidyl)ethyl]carbamate instead of tert-butyl 3,9-diazaspiro[5.5]undecane-3-carboxylate (compound 1e). Example 79 (26 mg) was obtained as a yellow solid. MS: calc'd 433 (MH⁺), measured 433 (MH⁺). ¹H NMR (400 MHz, METHANOL-d₄) δ=8.94-8.74 (m, 2H), 8.00 (d, J=8.6 Hz, 1H), 6.84 (d, J=8.6 Hz, 1H), 4.48-4.18 (m, 3H), 4.01-3.59 (m, 3H), 3.46 (br d, J=6.7 Hz, 2H), 3.25-2.83 (m, 5H), 2.03 (br d, J=12.5 Hz, 2H), 1.67 (br d, J=7.9 Hz, 6H).

### Example 80

### 8-[(3S,4R)-3-[[4-(dimethylamino)-1-piperidyl]methyl]-4-(trifluoromethyl)pyrrolidin-1-yl]quinoxaline-5-carbonitrile

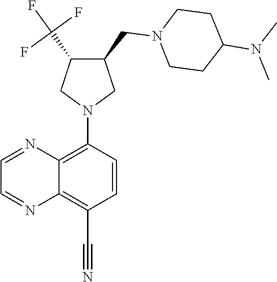 [SMILES: CN(C)C1CCN(C[C@H]2CN(c3ccc(C#N)c4nccnc34)C[C@@H]2C(F)(F)F)CC1]

The title compound was prepared in analogy to the preparation of Example 1 by using N,N-dimethylpiperidin-4-amine instead of tert-butyl 3,9-diazaspiro[5.5]undecane-3-carboxylate (compound 1e). Example 80 (19 mg) was obtained as a yellow solid. MS: calc'd 433 (MH⁺), measured 433 (MH⁺). ¹H NMR (400 MHz, METHANOL-d₄) δ=8.96-8.68 (m 2H), 7.98 (d, J=8.6 Hz, 1H), 6.82 (d, J=8.7 Hz, 1H), 4.48-4.19 (m, 3H), 3.98-3.61 (m, 3H), 3.62-3.38 (m, 1H), 3.15-2.78 (m, 10H), 2.49-1.92 (m, 4H).

### Example 81

### 8-[(3S,4R)-3-[[4-(methylaminomethyl)-1-piperidyl]methyl]-4-(trifluoromethyl)pyrrolidin-1-yl]quinoxaline-5-carbonitrile

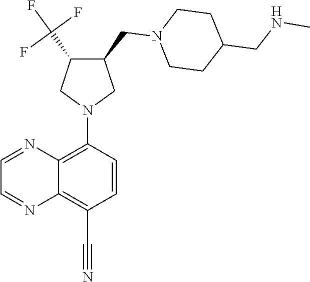 [SMILES: CNCC1CCN(C[C@H]2CN(c3ccc(C#N)c4nccnc34)C[C@@H]2C(F)(F)F)CC1]

The title compound was prepared in analogy to the preparation of Example 1 by using tert-butyl N-methyl-N-(4-piperidylmethyl)carbamate instead of tert-butyl 3,9-diazaspiro[5.5]undecane-3-carboxylate (compound 1e). Example 81 (32 mg) was obtained as a yellow solid. MS: calc'd 433 (MH⁺), measured 433 (MH⁺). ¹H NMR (400 MHz, METHANOL-d₄) δ=8.97-8.71 (m, 2H), 8.00 (dd, J=4.0, 8.6 Hz, 1H), 6.84 (dd, J=3.5, 8.4 Hz, 1H), 4.51-4.18 (m, 3H), 3.99-3.63 (m, 3H), 3.56-3.39 (m, 2H), 3.21-2.89 (m, 6H), 2.56 (s, 3H), 2.08 (br d, J=12.1 Hz, 3H), 1.71 (br d, J=12.1 Hz, 2H).

### Example 82

### 8-[(3S,4R)-3-[[(3S)-3-[(dimethylamino)methyl]pyrrolidin-1-yl]methyl]-4-(trifluoromethyl)pyrrolidin-1-yl]quinoxaline-5-carbonitrile

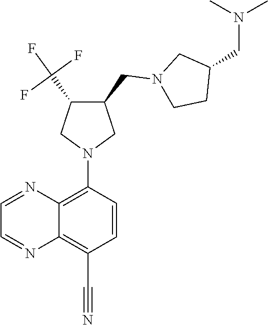 [SMILES: CN(C)C[C@@H]1CCN(C[C@H]2CN(c3ccc(C#N)c4nccnc34)C[C@@H]2C(F)(F)F)C1]

The title compound was prepared in analogy to the preparation of Example 1 by using N,N-dimethyl-1-[(3R)-pyrrolidin-3-yl]methanamine instead of tert-butyl 3,9-diazaspiro[5.5]undecane-3-carboxylate (compound 1e). Example 82 (12 mg) was obtained as a yellow solid. MS: calc'd 433 (MH⁺), measured 433 (MH⁺). ¹H NMR (400 MHz, METHANOL-d₄) δ=8.87 (d, J=1.7 Hz, 1H), 8.79 (d, J=1.7 Hz, 1H), 7.99-7.85 (m, 1H), 6.80 (d, J=8.7 Hz, 1H), 4.52-4.20 (m, 3H), 3.95 (dd, J=6.8, 11.5 Hz, 1H), 3.92-3.50 (m, 7H), 3.21-3.00 (m, 4H), 2.97 (s, 6H), 2.61-2.34 (m, 1H), 2.10-1.84 (m, 1H)

### Example 83

### 8-[(3S,4R)-3-[[(3R)-3-(dimethylamino)pyrrolidin-1-yl]methyl]-4-(trifluoromethyl)pyrrolidin-1-yl]quinoxaline-5-carbonitrile

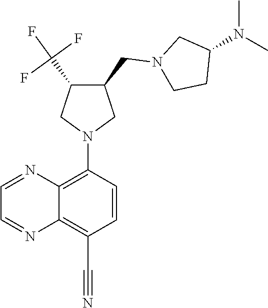 [SMILES: CN(C)[C@@H]1CCN(C[C@H]2CN(c3ccc(C#N)c4nccnc34)C[C@@H]2C(F)(F)F)C1]

The title compound was prepared in analogy to the preparation of Example 1 by using (3R)—N,N-dimethylpyrrolidin-3-amine instead of tert-butyl 3,9-diazaspiro[5.5]undecane-3-carboxylate (compound 1e). Example 83 (23 mg) was obtained as a yellow solid. MS: calc'd 419 (MH⁺), measured 419 (MH⁺). ¹H NMR (400 MHz, METHANOL-d₄) δ=8.88 (d, J=1.7 Hz, 1H), 8.79 (d, J=1.7 Hz, 1H), 7.98 (d, J=8.6 Hz, 1H), 6.82 (d, J=8.7 Hz, 1H), 4.43 (dd, J=8.5, 12.5 Hz, 1H), 4.27 (dt, J=6.5, 12.2 Hz, 2H), 4.07-3.96 (m, 1H), 3.88 (dd, J=5.4, 11.6 Hz, 1H), 3.52 (br s, 1H), 3.30-3.17 (m, 1H), 3.17-2.99 (m, 4H), 2.97-2.83 (m, 8H), 2.57-2.42 (m, 1H), 2.29-2.12 (m, 1H).

### Example 84

### 8-[(3S,4R)-3-[(7-methyl-2,7-diazaspiro[4.4]nonan-2-yl)methyl]-4-(trifluoromethyl)pyrrolidin-1-yl]quinoxaline-5-carbonitrile

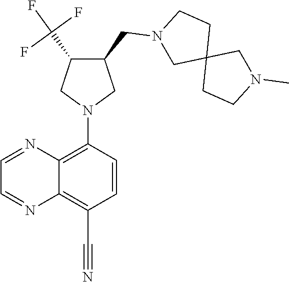 [SMILES: CN1CCC2(CCN(C[C@H]3CN(c4ccc(C#N)c5nccnc45)C[C@@H]3C(F)(F)F)C2)C1]

The title compound was prepared in analogy to the preparation of Example 1 by using 2-methyl-2,7-diazaspiro[4.4]nonane instead of tert-butyl 3,9-diazaspiro[5.5]undecane-3-carboxylate (compound 1e). Example 84 (7 mg) was obtained as a yellow solid. MS: calc'd 445 (MH⁺), measured 445 (MH⁺). ¹H NMR (400 MHz, METHANOL-d₄) δ=8.87 (s, 1H), 8.79 (s, 1H), 7.96 (br d, J=8.6 Hz, 1H), 6.80 (br d, J=8.7 Hz, 1H), 4.46-4.22 (m, 4H), 3.95 (br dd, J=6.7, 11.3 Hz, 2H), 3.79-3.49 (m, 9H), 3.19-3.06 (m, 1H), 3.01 (s, 3H), 2.36 (br s, 4H).

### Example 85

### 8-[(3S,4R)-3-[[3-(aminomethyl)-3-methyl-pyrrolidin-1-yl]methyl]-4-(trifluoromethyl)pyrrolidin-1-yl]quinoxaline-5-carbonitrile

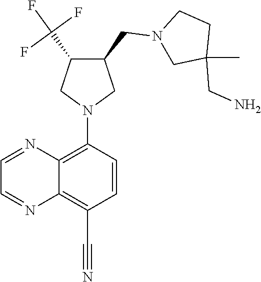 [SMILES: CC1(CN)CCN(C[C@H]2CN(c3ccc(C#N)c4nccnc34)C[C@@H]2C(F)(F)F)C1]

The title compound was prepared in analogy to the preparation of Example 1 by using tert-butyl N-[(3-methylpyrrolidin-3-yl)methyl]carbamate instead of tert-butyl 3,9-diazaspiro[5.5]undecane-3-carboxylate (compound 1e). Example 85 (13 mg) was obtained as a yellow solid. MS: calc'd 419 (MH⁺), measured 419 (MH⁺). ¹H NMR (400 MHz, METHANOL-d₄) δ=8.94-8.71 (m, 2H), 8.02-7.79 (m, 1H), 6.89-6.59 (m, 1H), 4.49-4.20 (m, 4H), 3.94 (br d, J=7.2 Hz, 1H), 3.64 (br m, 5H), 3.27-2.87 (m, 4H), 2.28-1.97 (m, 2H), 1.44-1.27 (m, 3H).

### Example 86

The following tests were carried out in order to determine the activity of the compounds of formula (I) in HEK293-Blue-hTLR-7/8/9 cells assay.

HEK293-Blue-hTLR-7 Cells Assay:

A stable HEK293-Blue-hTLR-7 cell line was purchased from InvivoGen (Cat. #: hkb-htlr7, San Diego, Calif., USA). These cells were originally designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was declined by TLR7 antagonist under the stimulation of a ligand, such as R848 (Resiquimod), for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR7 cells were incubated at a density of 250,000-450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620-655 nm using a spectrophotometer. The signalling pathway that TLR7 activation leads to downstream NF-κB activation has been widely accepted, and therefore similar reporter assay was modified for evaluating TLR7 antagonist.

HEK293-Blue-hTLR-8 Cells Assay:

A stable HEK293-Blue-hTLR-8 cell line was purchased from InvivoGen (Cat. #: hkb-htlr8, San Diego, Calif., USA). These cells were originally designed for studying the stimulation of human TLR8 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR8 cells with TLR8 ligands. Therefore the reporter expression was declined by TLR8 antagonist under the stimulation of a ligand, such as R848, for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR8 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 60 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620-655 nm using a spectrophotometer. The signalling pathway that TLR8 activation leads to downstream NF-κB activation has been widely accepted, and therefore similar reporter assay was modified for evaluating TLR8 antagonist.

HEK293-Blue-hTLR-9 Cells Assay:

A stable HEK293-Blue-hTLR-9 cell line was purchased from InvivoGen (Cat. #: hkb-htlr9, San Diego, Calif., USA). These cells were originally designed for studying the stimulation of human TLR9 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR9 cells with TLR9 ligands. Therefore the reporter expression was declined by TLR9 antagonist under the stimulation of a ligand, such as ODN2006 (Cat. #: tlrl-2006-1, Invivogen, San Diego, Calif., USA), for incubation of 20 hrs. The cell culture supernatant SEAP reporter activity was determined using QUANTI-Blue™ kit (Cat. #: rep-qb1, Invivogen, San Diego, Calif., USA) at a wavelength of 640 nm, a detection medium that turns purple or blue in the presence of alkaline phosphatase.

HEK293-Blue-hTLR9 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM ODN2006 in above DMEM, perform incubation under 37° C. in a CO₂incubator for 20 hrs. Then 20 μL of the superatant from each well was incubated with 180 μL Quanti-blue substrate solution at 37° C. for 2 hrs and the absorbance was read at 620˜655 nm using a spectrophotometer. The signalling pathway that TLR9 activation leads to downstream NF-κB activation has been widely accepted, and therefore similar reporter assay was modified for evaluating TLR9 antagonist.

The compounds of formula (I) have human TLR7 and/or TLR8 inhibitory activities (IC₅₀ value) <1 μM, particularly <0.1 μM. Moreover, some compounds also have human TLR9 inhibitory activity <1 μM, particularly <0.1 μM. Activity data of the compounds of the present invention were shown in Table 1.

TABLE 1
The activity of the compounds of present invention in
HEK293-Blue-hTLR-7/8/9 cells assays

| Example | TLR7 IC₅₀ (μM) | TLR8 IC₅₀ (μM) | TLR9 IC₅₀ (μM) |
|---|---|---|---|
| 1 | 0.005 | 0.020 | 0.106 |
| 2 | 0.020 | 0.057 | 0.076 |
| 3 | 0.027 | 0.012 | 1.868 |
| 4 | 0.028 | 0.038 | 0.244 |
| 5 | 0.036 | 0.035 | 0.137 |
| 6 | 0.048 | 0.039 | 0.136 |
| 7 | 0.054 | 0.043 | 0.077 |
| 8 | 0.057 | 0.055 | 0.113 |
| 9 | 0.061 | 0.047 | 0.104 |
| 10 | 0.069 | 0.052 | 0.834 |
| 11 | 0.074 | 0.024 | 0.125 |
| 12 | 0.015 | 0.031 | 0.219 |
| 13 | 0.102 | 0.050 | 0.06 |
| 14 | 0.043 | 0.019 | 0.153 |
| 15 | 0.107 | 0.190 | 0.032 |
| 16 | 0.113 | 0.133 | 0.05 |
| 17 | 0.128 | 0.065 | 0.134 |
| 18 | 0.137 | 0.082 | 0.097 |
| 19 | 0.142 | 0.253 | 0.815 |
| 20 | 0.156 | 0.068 | 5.641 |
| 21 | 0.189 | 0.351 | 0.294 |
| 22 | 0.201 | 0.079 | 0.153 |
| 23 | 0.251 | 0.266 | 0.045 |
| 24 | 0.298 | 0.063 | 0.082 |
| 25 | 0.311 | 0.108 | 0.075 |
| 26 | 0.055 | 0.021 | 0.258 |
| 27 | 0.354 | 0.454 | 0.032 |
| 28 | 0.355 | 0.948 | 0.039 |
| 29 | 0.370 | 0.197 | 0.056 |
| 30 | 0.382 | 0.072 | 0.307 |
| 31 | 0.385 | 0.418 | 0.383 |
| 32 | 0.402 | 0.111 | 0.080 |
| 33 | 0.432 | 0.245 | 0.863 |
| 34 | 0.454 | 0.373 | 0.085 |
| 35 | 0.468 | 0.245 | 0.050 |
| 36 | 0.471 | 5.170 | 0.050 |
| 37 | 0.651 | 0.531 | 0.032 |
| 39 | 0.709 | 0.132 | 0.097 |
| 40 | 0.746 | 0.742 | 0.685 |
| 41 | 0.821 | 0.357 | 1.197 |
| 42 | 0.867 | 0.161 | 0.153 |
| 43 | 0.899 | 0.718 | 0.062 |
| 44 | 0.963 | 0.533 | 0.091 |
| 45 | 0.049 | 0.032 | 0.224 |
| 46 | 0.093 | 0.067 | 0.275 |
| 47 | 0.088 | 0.074 | 0.177 |
| 48 | 0.051 | 0.037 | 0.097 |
| 49 | 0.153 | 0.024 | 0.087 |
| 50 | 0.232 | 0.038 | 0.259 |
| 51 | 0.090 | 0.057 | 0.359 |
| 52 | 0.039 | 0.088 | 0.208 |
| 53 | 0.115 | 0.146 | 0.219 |
| 54 | 0.091 | 0.044 | 0.152 |
| 55 | 0.111 | 0.042 | 0.173 |
| 56 | 0.113 | 0.062 | 0.159 |
| 57 | 0.073 | 0.085 | 0.331 |
| 58 | 0.313 | 0.091 | 0.605 |
| 59 | 0.018 | 0.014 | 2.696 |
| 60 | 0.029 | 0.043 | 0.509 |
| 61 | 0.022 | 0.023 | 0.438 |
| 62 | 0.034 | 0.028 | 0.409 |
| 63 | 0.057 | 0.025 | 0.294 |
| 64 | 0.036 | 0.086 | 23.111 |
| 65 | 0.108 | 0.092 | 0.068 |
| 66 | 0.063 | 0.082 | 0.123 |
| 67 | 0.131 | 0.041 | 0.869 |
| 68 | 0.075 | 0.051 | 0.093 |
| 68A | 0.061 | 0.060 | 0.071 |
| 69 | 0.135 | 0.729 | 15.712 |
| 70 | 0.012 | 0.020 | 0.253 |
| 71 | 0.039 | 0.094 | 0.087 |
| 72 | 0.013 | 0.023 | 0.506 |
| 73 | 0.020 | 0.035 | 1.191 |
| 74 | 0.028 | 0.055 | 0.325 |
| 75 | 0.026 | 0.031 | 0.767 |
| 76 | 0.021 | 0.041 | 0.485 |
| 77 | 0.013 | 0.026 | 2.337 |
| 78 | 0.032 | 0.030 | 1.915 |
| 79 | 0.037 | 0.102 | 0.248 |
| 80 | 0.014 | 0.032 | 0.706 |
| 81 | 0.032 | 0.025 | 0.218 |
| 82 | 0.106 | 0.108 | 0.304 |
| 83 | 0.025 | 0.065 | 2.015 |
| 84 | 0.048 | 0.057 | 0.409 |
| 85 | 0.083 | 0.075 | 0.314 |

1. A compound of formula (I), 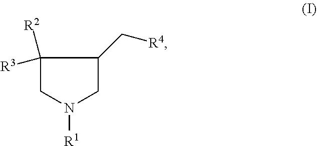
wherein
R¹ is 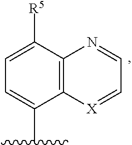
wherein
R⁵ is cyano, C₁₋₆alkyl, halogen, haloC₁₋₆alkyl or nitro;
X is N or CH;
R² and R³ are independently selected from H, C₁₋₆alkyl, C₃₋₇cycloalkyl and haloC₁₋₆alkyl; or R² and R³ together with the carbon they are attached to form C₃₋₇cycloalkyl;
R⁴ is heterocyclyl or heterocyclylamino;
or a pharmaceutically acceptable salt, enantiomer or diastereomer thereof.

2. A compound according to claim 1, wherein
R¹ is 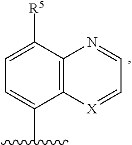
wherein
R⁵ is cyano or haloC₁₋₆alkyl;
X is N or CH;
R² is H;
R³ is H, C₁₋₆alkyl, C₃₋₇cycloalkyl or haloC₁₋₆alkyl;
or R² and R³ together with the carbon they are attached to form C₃₋₇cycloalkyl;
R⁴ is heterocyclyl or heterocyclylamino;
or a pharmaceutically acceptable salt, enantiomer or diastereomer thereof.

3. A compound according to claim 2, wherein
R⁴ is 2,3,3a,4,6,6a-hexahydro-1H-pyrrolo[3,4-c]pyrrolyl;
aminoazabicyclo[3.2.1]octanyl;
aminoazaspiro[3.3]heptanyl;
azepanylamino;
C₁₋₆alkyldiazaspiro[4.4]nonanyl;
diazabicyclo[2.2.2]octanyl;
diazabicyclo[4.2.0]octanyl;
diazaspiro[3.5]nonanyl;
diazaspiro[4.4]nonanyl;
diazaspiro[4.5]decanyl;
diazaspiro[5.5]undecanyl;
oxadiazaspiro[5.5]undecanyl;
piperazinyl;
piperidinyl, said piperidinyl being substituted by one, two or three substituents independently selected from amino, halogen, C₁₋₆alkyl, aminoC₁₋₆alkyl, (C₁₋₆alkyl)₂amino, C₁₋₆alkylaminoC₁₋₆alkyl, carbamoyl, azepanyl, morpholinyl, piperidinyl, piperazinyl and pyrrolidinyl;
piperidinylamino; or
pyrrolidinyl, said pyrrolidinyl being substituted by one, two or three substituents independently selected from C₁₋₆alkyl, (C₁₋₆alkyl)₂aminoC₁₋₆alkyl, (C₁₋₆alkyl)₂amino and aminoC₁₋₆alkyl.

4. A compound according to claim 3, wherein
R⁴ is 2,3,3a,4,6,6a-hexahydro-1H-pyrrolo[3,4-c]pyrrolyl; aminoazabicyclo[3.2.1]octanyl; aminoazaspiro[3.3]heptanyl; azepanylamino; C1-alkyldiazaspiro[4.4]nonanyl; diazabicyclo[2.2.2]octanyl; diazabicyclo[4.2.0]octanyl; diazaspiro[3.5]nonanyl; diazaspiro[4.4]nonanyl; diazaspiro[4.5]decanyl; diazaspiro[5.5]undecanyl; oxadiazaspiro[5.5]undecanyl; piperazinyl; amino(C-alkyl)piperidinyl; piperidinylpiperidinyl; aminopiperidinyl; piperazinylpiperidinyl; morpholinylpiperidinyl; pyrrolidinylpiperidinyl; azepanylpiperidinyl; aminohalopiperidinyl; carbamoylpiperidinyl; (aminoC₁₋₆alkyl)piperidinyl; aminoC₁₋₆alkyl(C₁₋₆alkyl)piperidinyl; (aminoC₁₋₆alkyl)halopiperidinyl; (C₁₋₆alkyl)₂aminopiperidinyl; C₁₋₆alkylaminoC₁₋₆alkylpiperidinyl; piperidinylamino; aminoC₁₋₆alkyl(C₁₋₆alkyl)pyrrolidinyl; (C₁₋₆alkyl)₂aminopyrrolidinyl; or (C₁₋₆alkyl)₂aminoC₁₋₆alkylpyrrolidinyl.

5. A compound according to claim 4, wherein R⁵ is cyano or trifluoromethyl.

6. A compound according to claim 5, wherein R³ is H, methyl, ethyl, isopropyl, difluoromethyl, trifluoromethyl or cyclopropyl; or R² and R³ together with the carbon they are attached to form cyclopropyl.

7. A compound according to claim 6, wherein R³ is methyl or trifluoromethyl; or R² and R³ together with the carbon they are attached to form cyclopropyl.

8. A compound according to claim 5 or 6, wherein R⁴ is (dimethylamino)methylpyrrolidinyl; (dimethylamino)pyrrolidinyl; 1,9-diazaspiro[5.5]undecan-9-yl; 1-oxa-4,9-diazaspiro[5.5]undecan-4-yl; 1-oxa-4,9-diazaspiro[5.5]undecan-9-yl; 2,3,3a,4,6,6a-hexahydro-1H-pyrrolo[3,4-c]pyrrol-5-yl; 2,5-diazabicyclo[2.2.2]octan-2-yl; 2,7-diazaspiro[3.5]nonan-2-yl; 2,7-diazaspiro[4.4]nonan-2-yl; 2,8-diazaspiro[3.5]nonan-2-yl; 2,8-diazaspiro[4.5]decan-2-yl; 2,8-diazaspiro[4.5]decan-8-yl; 2,9-diazaspiro[4.5]decan-2-yl; 2,9-diazaspiro[5.5]undecan-2-yl; 2,9-diazaspiro[5.5]undecan-9-yl; 3-(aminomethyl)-3-fluoro-1-piperidinyl; 3-(aminomethyl)-3-methyl-1-piperidinyl; 3-(aminomethyl)-3-methyl-pyrrolidin-1-yl; 3,7-diazabicyclo[4.2.0]octan-3-yl; 3,8-diazabicyclo[4.2.0]octan-8-yl; 3,9-diazaspiro[5.5]undecan-3-yl; 3-amino-8-azabicyclo[3.2.1]octan-8-yl; 4-(1-piperidinyl)-1-piperidinyl; 4-(2-aminoethyl)-1-piperidinyl; 4-(aminomethyl)-1-piperidinyl; 4-(azepan-1-yl)-1-piperidinyl; 4-(dimethylamino)-1-piperidinyl; 4-(methylaminomethyl)-1-piperidinyl; 4-amino-1-piperidinyl; 4-amino-3,3-difluoro-1-piperidinyl; 4-amino-3-methyl-1-piperidinyl; 4-amino-4-methyl-1-piperidinyl; 4-morpholino-1-piperidinyl; 4-piperazin-1-yl-1-piperidinyl; 4-pyrrolidin-1-yl-1-piperidinyl; 6-amino-2-azaspiro[3.3]heptan-2-yl; 7-methyl-2,7-diazaspiro[4.4]nonan-2-yl; 8-amino-3-azabicyclo[3.2.1]octan-3-yl; azepan-4-ylamino; carbamoylpiperidinyl; piperazinyl or piperidinylamino.

9. A compound according to claim 8, wherein R⁴ is 2,7-diazaspiro[3.5]nonan-2-yl; 1,9-diazaspiro[5.5]undecan-9-yl; 4-amino-1-piperidinyl; 4-amino-1-piperidinyl or piperidinylamino.

10. A compound according to claim 3, selected from:
8-[(3S,4R)-3-(3,9-diazaspiro[5.5]undecan-3-ylmethyl)-4-(trifluoromethyl)pyrrolidin-1-yl]quinoxaline-5-carbonitrile;
5-[(3S,4R)-3-(3,9-diazaspiro[5.5]undecan-3-ylmethyl)-4-(trifluoromethyl)pyrrolidin-1-yl]quinoline-8-carbonitrile;
8-[7-(piperazin-1-ylmethyl)-5-azaspiro[2.4]heptan-5-yl]quinoxaline-5-carbonitrile;
8-[7-[(8-Amino-3-azabicyclo[3.2.1]octan-3-yl)methyl]-5-azaspiro[2.4]heptan-5-yl]quinoxaline-5-carbonitrile;
8-[(3S,4R)-3-(2,7-diazaspiro[4.4]nonan-2-ylmethyl)-4-(trifluoromethyl)pyrrolidin-1-yl]quinoxaline-5-carbonitrile;
5-[(3S,4R)-3-(2,9-diazaspiro[4.5]decan-2-ylmethyl)-4-(trifluoromethyl)pyrrolidin-1-yl]quinoline-8-carbonitrile;
8-[7-[(4-Amino-4-methyl-1-piperidyl)methyl]-5-azaspiro[2.4]heptan-5-yl]quinoxaline-5-carbonitrile;
5-[(3S,4R)-3-(2,8-diazaspiro[3.5]nonan-2-ylmethyl)-4-(trifluoromethyl)pyrrolidin-1-yl]quinoline-8-carbonitrile;
5-[(3S,4R)-3-(2,7-diazaspiro[4.4]nonan-2-ylmethyl)-4-(trifluoromethyl)pyrrolidin-1-yl]quinoline-8-carbonitrile;
5-[(3S,4R)-3-[[4-(1-piperidyl)-1-piperidyl]methyl]-4-(trifluoromethyl)pyrrolidin-1-yl]quinoline-8-carbonitrile;
8-[7-[[4-(1-Piperidyl)-1-piperidyl]methyl]-5-azaspiro[2.4]heptan-5-yl]quinoxaline-5-carbonitrile;
8-[(3S,4R)-3-[(4-amino-4-methyl-1-piperidyl)methyl]-4-(trifluoromethyl)pyrrolidin-1-yl]quinoxaline-5-carbonitrile;
8-[7-(2,9-Diazaspiro[5.5]undecan-9-ylmethyl)-5-azaspiro[2.4]heptan-5-yl]quinoxaline-5-carbonitrile;
8-[(3S,4R)-3-(2,8-Diazaspiro[4.5]decan-8-ylmethyl)-4-(trifluoromethyl)pyrrolidin-1-yl]quinoxaline-5-carbonitrile;
5-[Trans-3-(3,9-diazaspiro[5.5]undecan-3-ylmethyl)-4-ethyl-pyrrolidin-1-yl]quinoline-8-carbonitrile;
8-[7-(3,9-Diazaspiro[5.5]undecan-3-ylmethyl)-5-azaspiro[2.4]heptan-5-yl]quinoxaline-5-carbonitrile;
8-[(3S,4R)-3-(3,9-diazaspiro[5.5]undecan-3-ylmethyl)-4-methyl-pyrrolidin-1-yl]quinoxaline-5-carbonitrile;
8-[7-[[(3aR,6aS)-2,3,3a,4,6,6a-hexahydro-1H-pyrrolo[3,4-c]pyrrol-5-yl]methyl]-5-azaspiro[2.4]heptan-5-yl]quinoxaline-5-carbonitrile;
5-[Trans-3-(2,7-diazaspiro[4.4]nonan-2-ylmethyl)-4-(trifluoromethyl)pyrrolidin-1-yl]-8-(trifluoromethyl)quinoxaline;
5-[7-(Piperazin-1-ylmethyl)-5-azaspiro[2.4]heptan-5-yl]-8-(trifluoromethyl)quinoxaline;
3-[[Trans-4-methyl-1-[8-(trifluoromethyl)quinoxalin-5-yl]pyrrolidin-3-yl]methyl]-3,9-diazaspiro[5.5]undecane;
8-[7-(2,9-Diazaspiro[4.5]decan-2-ylmethyl)-5-azaspiro[2.4]heptan-5-yl]quinoxaline-5-carbonitrile;
5-[(3S,4R)-3-(3,9-diazaspiro[5.5]undecan-3-ylmethyl)-4-methyl-pyrrolidin-1-yl]quinoline-8-carbonitrile;
8-[7-(2,8-Diazaspiro[3.5]nonan-2-ylmethyl)-5-azaspiro[2.4]heptan-5-yl]quinoxaline-5-carbonitrile;
8-[7-[(3-Amino-8-azabicyclo[3.2.1]octan-8-yl)methyl]-5-azaspiro[2.4]heptan-5-yl]quinoxaline-5-carbonitrile;
8-[(3S,4R)-3-[(4-Amino-1-piperidyl)methyl]-4-(trifluoromethyl)pyrrolidin-1-yl]quinoxaline-5-carbonitrile;
5-[Trans-3-(3,9-diazaspiro[5.5]undecan-3-ylmethyl)-4-(difluoromethyl)pyrrolidin-1-yl]quinoline-8-carbonitrile;
5-[Trans-3-cyclopropyl-4-(3,9-diazaspiro[5.5]undecan-3-ylmethyl)pyrrolidin-1-yl]quinoline-8-carbonitrile;
5-[Trans-3-(2,7-diazaspiro[4.4]nonan-2-ylmethyl)-4-ethyl-pyrrolidin-1-yl]quinoline-8-carbonitrile;
8-[(3S,4R)-3-(2,7-diazaspiro[4.4]nonan-2-ylmethyl)-4-methyl-pyrrolidin-1-yl]quinoxaline-5-carbonitrile;
3-[[5-[8-(Trifluoromethyl)quinoxalin-5-yl]-5-azaspiro[2.4]heptan-7-yl]methyl]-3,9-diazaspiro[5.5]undecane;
8-[7-(2,8-Diazaspiro[4.5]decan-2-ylmethyl)-5-azaspiro[2.4]heptan-5-yl]quinoxaline-5-carbonitrile;
5-[Trans-3-methyl-4-[[4-(1-piperidyl)-1-piperidyl]methyl]pyrrolidin-1-yl]-8-(trifluoromethyl)quinoxaline;
5-[Trans-3-(2,7-diazaspiro[4.4]nonan-2-ylmethyl)-4-(difluoromethyl)pyrrolidin-1-yl]quinoline-8-carbonitrile;
5-[(3S,4R)-3-(2,7-diazaspiro[4.4]nonan-2-ylmethyl)-4-methyl-pyrrolidin-1-yl]quinoline-8-carbonitrile;
5-[Trans-3-(3,9-diazaspiro[5.5]undecan-3-ylmethyl)-4-isopropyl-pyrrolidin-1-yl]quinoline-8-carbonitrile;
8-[3-(3,9-Diazaspiro[5.5]undecan-3-ylmethyl)pyrrolidin-1-yl]quinoxaline-5-carbonitrile;
5-[7-(3,9-Diazaspiro[5.5]undecan-3-ylmethyl)-5-azaspiro[2.4]heptan-5-yl]quinoline-8-carbonitrile;
8-[7-[(Azepan-4-ylamino)methyl]-5-azaspiro[2.4]heptan-5-yl]quinoxaline-5-carbonitrile;
N-[Trans-4-methyl-1-[8-(trifluoromethyl)quinoxalin-5-yl]pyrrolidin-3-yl]methyl]azepan-4-amine;
5-[7-[[(3aR,6aS)-2,3,3a,4,6,6a-hexahydro-1H-pyrrolo[3,4-c]pyrrol-5-yl]methyl]-5-azaspiro[2.4]heptan-5-yl]-8-(trifluoromethyl)quinoxaline;
8-[7-[(6-Amino-2-azaspiro[3.3]heptan-2-yl)methyl]-5-azaspiro[2.4]heptan-5-yl]quinoxaline-5-carbonitrile;
5-[7-(2,7-Diazaspiro[4.4]nonan-2-ylmethyl)-5-azaspiro[2.4]heptan-5-yl]quinoline-8-carbonitrile;
5-[Trans-3-(2,7-diazaspiro[4.4]nonan-2-ylmethyl)-4-isopropyl-pyrrolidin-1-yl]quinoline-8-carbonitrile;
8-[(3S,4R)-3-(2,9-diazaspiro[5.5]undecan-9-ylmethyl)-4-(trifluoromethyl)pyrrolidin-1-yl]quinoxaline-5-carbonitrile;
8-[(3S,4R)-3-(2,8-diazaspiro[4.5]decan-2-ylmethyl)-4-(trifluoromethyl)pyrrolidin-1-yl]quinoxaline-5-carbonitrile;
8-[(3S,4R)-3-[(6-amino-2-azaspiro[3.3]heptan-2-yl)methyl]-4-(trifluoromethyl)pyrrolidin-1-yl]quinoxaline-5-carbonitrile;
8-[(3S,4R)-3-(2,7-diazaspiro[3.5]nonan-2-ylmethyl)-4-(trifluoromethyl)pyrrolidin-1-yl]quinoxaline-5-carbonitrile;
8-[7-(1-Oxa-4,9-diazaspiro[5.5]undecan-9-ylmethyl)-5-azaspiro[2.4]heptan-5-yl]quinoxaline-5-carbonitrile;
8-[7-(3,8-Diazabicyclo[4.2.0]octan-8-ylmethyl)-5-azaspiro[2.4]heptan-5-yl]quinoxaline-5-carbonitrile;
8-[(3S,4R)-3-[(4-amino-1-piperidyl)methyl]-4-(trifluoromethyl)pyrrolidin-1-yl]quinoxaline-5-carbonitrile;
5-[(3S,4R)-3-[(4-piperazin-1-yl-1-piperidyl)methyl]-4-(trifluoromethyl)pyrrolidin-1-yl]quinoline-8-carbonitrile;
5-[(3S,4R)-3-[(6-Amino-2-azaspiro[3.3]heptan-2-yl)methyl]-4-(trifluoromethyl)pyrrolidin-1-yl]quinoline-8-carbonitrile;
5-[(3S,4R)-3-(2,8-Diazaspiro[4.5]decan-8-ylmethyl)-4-(trifluoromethyl)pyrrolidin-1-yl]quinoline-8-carbonitrile;
5-[(3S,4R)-3-(2,9-Diazaspiro[5.5]undecan-9-ylmethyl)-4-(trifluoromethyl)pyrrolidin-1-yl]quinoline-8-carbonitrile;
5-[(3S,4R)-3-(2,8-Diazaspiro[4.5]decan-2-ylmethyl)-4-(trifluoromethyl)pyrrolidin-1-yl]quinoline-8-carbonitrile;
5-[(3S,4R)-3-(2,9-Diazaspiro[5.5]undecan-2-ylmethyl)-4-(trifluoromethyl)pyrrolidin-1-yl]quinoline-8-carbonitrile;
8-[7-[(4-Morpholino-1-piperidyl)methyl]-5-azaspiro[2.4]heptan-5-yl]quinoxaline-5-carbonitrile;
8-[(3S,4R)-3-(Piperazin-1-ylmethyl)-4-(trifluoromethyl)pyrrolidin-1-yl]quinoxaline-5-carbonitrile;
8-[(3S,4R)-3-[(4-Pyrrolidin-1-yl-1-piperidyl)methyl]-4-(trifluoromethyl)pyrrolidin-1-yl]quinoxaline-5-carbonitrile;
8-[(3S,4R)-3-[[4-(1-Piperidyl)-1-piperidyl]methyl]-4-(trifluoromethyl)pyrrolidin-1-yl]quinoxaline-5-carbonitrile;
8-[(3S,4R)-3-[[4-(Azepan-1-yl)-1-piperidyl]methyl]-4-(trifluoromethyl)pyrrolidin-1-yl]quinoxaline-5-carbonitrile;
8-[(3S,4R)-3-[(3-amino-8-azabicyclo[3.2.1]octan-8-yl)methyl]-4-(trifluoromethyl)pyrrolidin-1-yl]quinoxaline-5-carbonitrile;
8-[(3S,4R)-3-[(4-amino-3,3-difluoro-1-piperidyl)methyl]-4-(trifluoromethyl)pyrrolidin-1-yl]quinoxaline-5-carbonitrile;
8-[7-(1,9-Diazaspiro[5.5]undecan-9-ylmethyl)-5-azaspiro[2.4]heptan-5-yl]quinoxaline-5-carbonitrile;
8-[7-(3,7-Diazabicyclo[4.2.0]octan-3-ylmethyl)-5-azaspiro[2.4]heptan-5-yl]quinoxaline-5-carbonitrile;
8-[7-(1-Oxa-4,9-diazaspiro[5.5]undecan-4-ylmethyl)-5-azaspiro[2.4]heptan-5-yl]quinoxaline-5-carbonitrile;
8-[7-[(4-Amino-1-piperidyl)methyl]-5-azaspiro[2.4]heptan-5-yl]quinoxaline-5-carbonitrile;
1-[[(3S,4R)-1-(8-cyanoquinoxalin-5-yl)-4-(trifluoromethyl)pyrrolidin-3-yl]methyl]piperidine-3-carboxamide;
8-[(3S,4R)-3-(1-oxa-4,9-diazaspiro[5.5]undecan-9-ylmethyl)-4-(trifluoromethyl)pyrrolidin-1-yl]quinoxaline-5-carbonitrile;
8-[(3S,4R)-3-[(4-piperidylamino)methyl]-4-(trifluoromethyl)pyrrolidin-1-yl]quinoxaline-5-carbonitrile;
8-[(3S,4R)-3-[[(3S,4R)-4-amino-3-methyl-1-piperidyl]methyl]-4-(trifluoromethyl)pyrrolidin-1-yl]quinoxaline-5-carbonitrile;
8-[(3S,4R)-3-[(8-amino-3-azabicyclo[3.2.1]octan-3-yl)methyl]-4-(trifluoromethyl)pyrrolidin-1-yl]quinoxaline-5-carbonitrile;
8-[(3S,4R)-3-[[4-(aminomethyl)-1-piperidyl]methyl]-4-(trifluoromethyl)pyrrolidin-1-yl]quinoxaline-5-carbonitrile;
8-[(3S,4R)-3-[[3-(aminomethyl)-3-methyl-1-piperidyl]methyl]-4-(trifluoromethyl)pyrrolidin-1-yl]quinoxaline-5-carbonitrile;
8-[(3S,4R)-3-(2,3,3a,4,6,6a-hexahydro-1H-pyrrolo[3,4-c]pyrrol-5-ylmethyl)-4-(trifluoromethyl)pyrrolidin-1-yl]quinoxaline-5-carbonitrile;
8-[(3S,4R)-3-(2,5-diazabicyclo[2.2.2]octan-2-ylmethyl)-4-(trifluoromethyl)pyrrolidin-1-yl]quinoxaline-5-carbonitrile;
8-[(3S,4R)-3-[[3-(aminomethyl)-3-fluoro-1-piperidyl]methyl]-4-(trifluoromethyl)pyrrolidin-1-yl]quinoxaline-5-carbonitrile;
8-[(3S,4R)-3-[[4-(2-aminoethyl)-1-piperidyl]methyl]-4-(trifluoromethyl)pyrrolidin-1-yl]quinoxaline-5-carbonitrile;
8-[(3S,4R)-3-[[4-(dimethylamino)-1-piperidyl]methyl]-4-(trifluoromethyl)pyrrolidin-1-yl]quinoxaline-5-carbonitrile;
8-[(3S,4R)-3-[[4-(methylaminomethyl)-1-piperidyl]methyl]-4-(trifluoromethyl)pyrrolidin-1-yl]quinoxaline-5-carbonitrile;
8-[(3S,4R)-3-[[(3S)-3-[(dimethylamino)methyl]pyrrolidin-1-yl]methyl]-4-(trifluoromethyl)pyrrolidin-1-yl]quinoxaline-5-carbonitrile;
8-[(3S,4R)-3-[[(3R)-3-(dimethylamino)pyrrolidin-1-yl]methyl]-4-(trifluoromethyl)pyrrolidin-1-yl]quinoxaline-5-carbonitrile;
8-[(3S,4R)-3-[(7-methyl-2,7-diazaspiro[4.4]nonan-2-yl)methyl]-4-(trifluoromethyl)pyrrolidin-1-yl]quinoxaline-5-carbonitrile; and
8-[(3S,4R)-3-[[3-(aminomethyl)-3-methyl-pyrrolidin-1-yl]methyl]-4-(trifluoromethyl)pyrrolidin-1-yl]quinoxaline-5-carbonitrile;
or a pharmaceutically acceptable salt, enantiomer or diastereomer thereof.

11. A compound according to claim 10, selected from:
5-[(3S,4R)-3-(3,9-diazaspiro[5.5]undecan-3-ylmethyl)-4-(trifluoromethyl)pyrrolidin-1-yl]quinoline-8-carbonitrile;
8-[7-[(4-Amino-4-methyl-1-piperidyl)methyl]-5-azaspiro[2.4]heptan-5-yl]quinoxaline-5-carbonitrile;
8-[7-(2,9-Diazaspiro[5.5]undecan-9-ylmethyl)-5-azaspiro[2.4]heptan-5-yl]quinoxaline-5-carbonitrile;
8-[(3S,4R)-3-(2,7-diazaspiro[3.5]nonan-2-ylmethyl)-4-(trifluoromethyl)pyrrolidin-1-yl]quinoxaline-5-carbonitrile;
8-[7-(1,9-Diazaspiro[5.5]undecan-9-ylmethyl)-5-azaspiro[2.4]heptan-5-yl]quinoxaline-5-carbonitrile;
8-[7-[(4-Amino-1-piperidyl)methyl]-5-azaspiro[2.4]heptan-5-yl]quinoxaline-5-carbonitrile; and
8-[(3S,4R)-3-[(4-piperidylamino)methyl]-4-(trifluoromethyl)pyrrolidin-1-yl]quinoxaline-5-carbonitrile;
or a pharmaceutically acceptable salt, enantiomer or diastereomer thereof.

12. A process for the preparation of a compound according to any one of claims 1 to 11 comprising the following step:
a) reaction of compound of formula (VI), 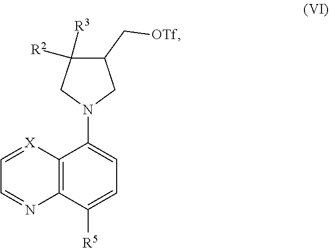
with amine (VII) in the presence of a base;
wherein base is Cs₂CO₃; R², R³, R⁵ and X are defined as in any one of claims 1 to 9.

13. A compound or pharmaceutically acceptable salt, enantiomer or diastereomer according to any one of claims 1 to 11 for use as therapeutically active substance.

14. A pharmaceutical composition comprising a compound in accordance with any one of claims 1 to 11 and a therapeutically inert carrier.

15. The use of a compound according to any one of claims 1 to 11 for the treatment or prophylaxis of systemic lupus erythematosus or lupus nephritis.

16. The use of a compound according to any one of claims 1 to 11 for the preparation of a medicament for the treatment or prophylaxis of systemic lupus erythematosus or lupus nephritis.

17. The use of a compound according to any one of claims 1 to 11 as the TLR7 or TLR8 or TLR9 antagonist.

18. The use of a compound according to any one of claims 1 to 11 as the TLR7 and TLR8 and TLR9 antagonist.

19. A compound or pharmaceutically acceptable salt, enantiomer or diastereomer according to any one of claims 1 to 11 for the treatment or prophylaxis of systemic lupus erythematosus or lupus nephritis.

20. A compound or pharmaceutically acceptable salt, enantiomer or diastereomer according to any one of claims 1 to 11, when manufactured according to a process of claim 12.

21. A method for the treatment or prophylaxis of systemic lupus erythematosus or lupus nephritis, which method comprises administering a therapeutically effective amount of a compound as defined in any one of claims 1 to 11.

22. The invention as hereinbefore described.
